(19) United States
(12) Patent Application Publication
(10) Pub. No.: US 2023/0147683 A1
(43) Pub. Date: May 11, 2023

(54) TREATMENT

(71) Applicant: UNION Therapeutics A/S, Hellerup (DK)

(72) Inventors: Morten SOMMER, Hellerup (DK); Rasmus TOFT-KEHLER, Hellerup (DK); Anne Katrine TOFT-KEHLER, Hellerup (DK); Mads JELLINGSOE, Hellerup (DK); Philippe ANDRES, Hellerup (DK)

(21) Appl. No.: 17/995,079

(22) PCT Filed: Mar. 26, 2021
(86) PCT No.: PCT/EP2021/058046

§ 371 Date: Sep. 29, 2022

(30) Foreign Application Priority Data
Apr. 1, 2020 (GB) 2004844.3
Apr. 9, 2020 (GB) 2005340.1
Jul. 9, 2020 (GB) 2010573.0
Jul. 9, 2020 (GB) 2010575.5
Oct. 14, 2020 (GB) 2016274.9
Oct. 14, 2020 (GB) 2016289.7
Mar. 22, 2021 (GB) 2103957.3

(51) Int. Cl. A61K 31/609; A61K 47/10; A61K 47/22; A61K 31/167; A61K 9/00; A61P 31/14; A61K 47/32; A61K 47/40; A61K 47/20; A61K 47/00; A61K 9/08

(52) U.S. Cl. CPC A61K 31/609; A61K 47/10; A61K 47/22; A61K 31/167; A61K 9/0078; A61P 31/14; A61K 47/32; A61K 47/40; A61K 47/20; A61K 47/00; A61K 9/08; A61K 9/0043

(57) ABSTRACT

Inhalable compositions comprising niclosamide, or a pharmaceutically acceptable salt thereof, and polyethylene glycol; aerosols of the compositions; and the pulmonary administrations for use in the treatment of viral infections, for example respiratory viral infections.

This invention relates to inhalable pharmaceutical compositions comprising niclosamide, or a pharmaceutically acceptable salt thereof, and their use in the treatment of viral infections, particularly pulmonary viral infections.

### BACKGROUND

Coronaviruses are a group of enveloped and non-segmented positive-sense RNA viruses with very large genome size ranging from approximately 27 to 34 kb. Infections with human strains HCoV-229E, HCoV-OC43, HCoV-NL63 and HCoV-HKU1 usually cause mild, self limiting respiratory infections, such as the common cold (Fehr et al. Coronaviruses: Methods and Protocols, Maier, H. J.; Bickerton, E.; Britton, P., Eds. Springer New York: New York, N.Y., 2015; pp 1-23 2015 and Corman et al., Adv. Virus Res., J., Eds. Academic Press: 2018; Vol. 100, pp 163-188 2018). However certain highly pathogenic coronaviruses have emerged. SARS-CoV, MERS-CoV and SARS-CoV-2, have caused severe human disease pandemics associated with high morbidity and mortality.

The lack of effective treatment for coronavirus infections poses a great challenge to clinical management and highlights the urgent need to fine new treatments for viral infections such as coronavirus infections.

Wang et al. (Remdesivir and chloroquine effectively inhibit the recently emerged novel coronavirus (2019-nCoV) in vitro. Cell Res. 2020, https://doi.org/10.1038/s41422-020-0282-0) screened antiviral drugs and identified that nitazoxanide, remdesivir and chloroquine, inhibit the SARS-CoV-2 at low-micromolar concentrations in Vero E6 cells with EC50 values of 2.12 μm, 0.77 μm and 1.13 μm, respectively.

Wu et al. (Inhibition of severe acute respiratory syndrome coronavirus replication by niclosamide, Antimicrob. Agents Chemother. 2004, 48, 2693-2696) found that niclosamide inhibits SARS-CoV replication and totally abolished viral antigen synthesis at a concentration of 1.56 μm. Niclosamide suppressed cytopathic effect (CPE) of SARS-CoV at concentration as low as 1 μm and inhibited SARS-CoV replication with an EC50 value of less than 0.1 μm in Vero E6 cells (Wen et al., J. Med. Chem. 2007, 50, 4087-4095.). Niclosamide was later found be a very potent inhibitor of SARS-CoV2 with an IC50 of 280 nM (Joun et al. Clinical features of patients infected with 2019 novel coronavirus in Wuhan, China, The Lancet, https://www.thelancet.com/journals/lancet/article/PIIS0140-6736(20)30183-5/fulltext).

Xu et al. (ACS Infect. Dis. 2020, published on line 3 Mar. 2020 https://doi.org/10.1021/acsinfecdis.0c00052) discloses that niclosamide is effective against certain viral infections. However, this publication concludes that the, low aqueous solubility, poor absorption and low oral bioavailability would limit its clinical development as an antiviral agent.

Cabitra et al., JCI Insight. 2019; 4(15):e128414 discloses the treatment of mice using niclosamide dissolved in corn oil and administered by I.P. injection showed that niclosamide reduced mucus production and secretion, as well as bronchoconstriction, and showed additional anti-inflammatory effects in asthmatic mice.

Niclosamide (tradenames are for instance Yomesan®, Tredemine®) is currently approved and marketed for the oral treatment of tapeworm infections with administration of a single 2 g regimen or 2 g daily for 7 days in adults and children (>2 years of age). The PK analysis revealed that after oral administration, between 2-25% of the administered dose was detected in the urine, which can be considered as the minimum level of absorption. When treating human volunteers each with a single oral dose of 2,000 mg niclosamide, maximal serum concentration of niclosamide was equivalent to 0.25-6.0 μg/mL (0.76-18.3 μM). The wide concentration range was caused by the intraindividual absorption differences. Niclosamide is only partially absorbed from intestinal tract, and the absorbed part is rapidly eliminated by the kidneys. Niclosamide has several other weaknesses such as low absorption and oral bioavailability (F=10%) which may hamper its extensive clinical development as a systemic agent.

WO 2017/157997 discloses certain compositions comprising niclosamide for the topical treatment of conditions such as atopic dermatitis.

There remains a need to identify effective treatments for viral infections.

### BRIEF SUMMARY OF THE DISCLOSURE

In accordance with the present inventions there is provided an inhalable pharmaceutical composition comprising niclosamide, or a pharmaceutically acceptable salt thereof, for use in the prevention or treatment of a viral infection in a subject, wherein the niclosamide, or a pharmaceutically acceptable salt thereof, is administered to the subject by inhalation.

Also provided is a pharmaceutical composition comprising niclosamide, or a pharmaceutically acceptable salt thereof, for use in the prevention or treatment of a viral infection in a subject, wherein the niclosamide, or a pharmaceutically acceptable salt thereof, is administered to the subject intranasally.

The pharmaceutical composition comprising niclosamide or a pharmaceutically acceptable salt thereof is in a form suitable for pulmonary administration. For example, it may be that the pharmaceutical composition comprising niclosamide or a pharmaceutically acceptable salt thereof is in the form of a powder, a suspension, an aerosol of the suspension, a solution or an aerosol of the solution.

It may be that the pharmaceutical composition is in the form of a suspension or solution comprising niclosamide or a pharmaceutically acceptable salt thereof, wherein the composition is administered to a subject as an aerosol of the solution or suspension. Preferably the niclosamide or a pharmaceutically acceptable salt thereof is administered to the subject in the form of an aerosol of a solution comprising niclosamide, or a pharmaceutically acceptable salt thereof. Solutions and suspensions comprising niclosamide, or a pharmaceutically acceptable salt thereof may be any of the solutions described herein.

In certain embodiments the pharmaceutical composition is a solution or suspension comprising niclosamide, or a pharmaceutically acceptable salt thereof. Thus it may be that the pharmaceutical composition is a suspension comprising niclosamide, or a pharmaceutically acceptable salt thereof. Preferably the pharmaceutical composition is a solution comprising niclosamide, or a pharmaceutically acceptable salt thereof.

It may be that the solution or suspension comprises niclosamide, or a pharmaceutically acceptable salt thereof, and polyethylene glycol (PEG), wherein the solution or suspension is administered to the subject by inhalation (e.g. wherein the composition is administered as an aerosol of the solution or suspension). It may be that the PEG has an average molecular weight of less than about 600. It may be that the PEG has an average molecular weight of from about 150 to about 600, for example an average molecular weight of about 200 or about 400. In preferred embodiments the PEG has a molecular weight of about 400.

It may be that the PEG is present in the solution or suspension comprising niclosamide, or a pharmaceutically acceptable salt thereof, in an amount of at least 25% by weight of the solution or suspension, for example, wherein the PEG is present in an amount of at least 30%, at least 35%, at least 40%, at least 45%, at least 50%, at least 55%, at least 60%, at least 65%, at least 70%, at least 75%, at least 80%, at least 85%, at least 90%, at least 95% or at least 98% by weight of the solution or suspension. For example it may be that the PEG is present in an amount of from about 40% to about 99% by weight of the solution or suspension. It may be that the PEG is present in an amount of from about 50% to about 98% by weight of the solution or suspension. It may be that the PEG is present in an amount of from about 55% to about 98%, by weight of the solution or suspension. It may be that the PEG is present in an amount of from about 60% to about 98% by weight of the solution or suspension. It may be that the PEG is present in an amount of from about 65% to about 98% by weight of the solution or suspension. It may be that the PEG is present in an amount of from about 70% to about 98% by weight of the solution or suspension. It may be that the PEG is present in an amount of from about 75% to about 98% by weight of the solution or suspension. It may be that the PEG is present in an amount of from about 80% to about 98% by weight of the solution or suspension. It may be that the PEG is present in an amount of from about 85% to about 98% by weight of the solution or suspension. It may be that the PEG is present in an amount of from about 75% to about 96% by weight of the solution or suspension. It may be that the PEG is present in an amount of from about 80% to about 96% by weight of the solution or suspension. It may be that the PEG is present in an amount of from about 85% to about 96% by weight of the solution or suspension. It may be that the PEG is present in an amount of from about 90% to about 96% by weight of the solution or suspension. It may be that the PEG is present in an amount of from about 93% to about 96% by weight of the solution or suspension. It may be that the PEG is present in an amount of about 90%, about 91% about 92%, about 93%, about 94%, about 95%, about 96% about 97%, or about 98% by weight of the solution or suspension.

In preferred embodiments the solution comprises a solution of niclosamide ethanolamine in PEG. For example, a solution comprising niclosamide ethanolamine in PEG, wherein the PEG has an average molecular weight of from about 150 to about 600, more preferably the PEG has an average molecular weight of from about 200 to about 400. In certain preferred embodiments the solution comprises niclosamide ethanolamine in PEG 200. In certain preferred embodiments the solution comprises niclosamide ethanolamine in PEG 400.

Preferably the solutions and suspensions comprising comprises niclosamide, or a pharmaceutically acceptable salt thereof, are liquid solutions or liquid suspensions.

Suitably when the inhalable pharmaceutical composition is a solution or suspension comprising niclosamide or a pharmaceutically acceptable salt thereof, the composition is administered to the subject by inhalation as an aerosol of the solution or suspension.

Accordingly in another aspect there is provided an aerosol of a solution comprising niclosamide, or a pharmaceutically acceptable salt thereof, for use in the prevention or treatment of a viral infection in a subject, wherein the aerosol is administered to the subject by inhalation.

In another aspect there is provided an aerosol of a solution comprising niclosamide, or a pharmaceutically acceptable salt thereof, for use in the prevention or treatment of a viral infection in a subject, wherein the aerosol is administered to the subject intranasally.

In another aspect there is provided an aerosol of a suspension comprising niclosamide, or a pharmaceutically acceptable salt thereof, for use in the prevention or treatment of a viral infection in a subject, wherein the aerosol is administered to the subject by inhalation.

In another aspect there is provided an aerosol of a suspension comprising niclosamide, or a pharmaceutically acceptable salt thereof, for use in the prevention or treatment of a viral infection in a subject, wherein the aerosol is administered to the subject intranasally.

The solution or suspension in the above aspects may be any of the solutions or suspensions disclosed herein. Preferably wherein the solution or suspension comprises niclosamide, or a pharmaceutically acceptable salt thereof and PEG.

In certain embodiments the inhalable pharmaceutical composition (e.g. solution, the suspension, an aerosol of the solution or suspension, or powder) comprises niclosamide in the free acid form.

In certain embodiments the inhalable pharmaceutical composition (e.g. solution, the suspension, an aerosol of the solution or suspension, or powder) comprises a pharmaceutically acceptable salt of niclosamide, for example niclosamide ethanolamine.

In certain embodiments the niclosamide, or a pharmaceutically acceptable salt thereof, is present in an amount of about 0.01% to about 10% by weight of the inhalable pharmaceutical composition. For example, the niclosamide or a pharmaceutically acceptable salt thereof is present in an amount of 0.05% to 10, 0.1% to 9%, 0.2% to 8.5%, 0.05% to 8%, 0.5% to 8%, 1% to 8%, 1.5% to 8%, 2% to 8%, 2.5% to 8%, 3% to 8%, 3.5% to 8%, 4% to 8%, 4.5% to 8%, 5% to 8%, 5.5% to 8%, 6% to 8%, 3% to 7%, 3.5% to 7.5%, 3.5% to 7%, 3.5% to 6.5%, 3.5% to 6%, 3.5% to 5.5%, 4% to 7%, 4% to 7%, 4% to 6.5%, 4% to 6%, 4% to 5.5%, 4.5% to 7%, 4.5% to 6.5%, 4.5% to 6.5% or 4.5% to 5.5% by weight of the inhalable pharmaceutical composition. In a preferred embodiment the niclosamide or a pharmaceutically acceptable salt thereof is present in the inhalable pharmaceutical composition in an amount of about 4.5% to 5.5% by weight of the composition. Thus it may be that the niclosamide or a pharmaceutically acceptable salt is present in the inhalable pharmaceutical composition in an amount of about 3.5% about 4%, about 4.5%, about 5%, about 5.5%, about 6%, about 6.5%, about 7%, about 7.5%, or about, 8% by weight of the inhalable pharmaceutical composition. In a preferred embodiment the inhalable composition comprises about 5% by weight of niclosamide or a pharmaceutically acceptable salt thereof. The amounts of niclosamide present in the inhalable composition is applicable to any of the compositions comprising niclosamide described herein, for example a solution comprising niclosamide or a pharmaceutically acceptable salt thereof; a suspension comprising niclosamide or a pharmaceutically acceptable salt thereof; an aerosol of a solution comprising niclosamide or a pharmaceutically acceptable salt thereof; an aerosol of a suspension comprising niclosamide or a pharmaceutically acceptable salt thereof; or a powder comprising niclosamide or a pharmaceutically acceptable salt thereof.

In certain embodiments the inhalable composition is a solution or suspension (preferably a solution) comprising from about 1% to about 10% by weight niclosamide ethanolamine and PEG, wherein the PEG has an average molecular weight of less than 600.

In certain embodiments the inhalable composition is a solution comprising from about 4.5% to about 6.5% by weight niclosamide ethanolamine and about 93.5% to about 95.5% by weight PEG 400

Niclosamide has a very low solubility in water and may be prone to precipitation when formulated in compositions that contain water. Accordingly, in certain embodiments the composition administered to the subject is a non-aqueous inhalable composition) comprising niclosamide or a pharmaceutically acceptable salt thereof. In certain embodiments it may be that the composition administered to the subject is a non-aqueous inhalable solution or suspension comprising niclosamide or a pharmaceutically acceptable salt thereof. In certain embodiments the niclosamide administered to the subject is in the form of an inhalable non-aqueous solution comprising niclosamide, or a pharmaceutically acceptable salt thereof. In certain embodiments the niclosamide administered to the subject is in the form of a non-aqueous inhalable solution comprising niclosamide. In certain embodiments the niclosamide administered to the subject is in the form of a non-aqueous inhalable solution comprising a pharmaceutically acceptable salt of niclosamide. In certain embodiments the niclosamide administered to the subject is in the form of a non-aqueous inhalable solution comprising niclosamide ethanolamine. Preferably the niclosamide administered to the subject is a non-aqueous solution comprising niclosamide, or a pharmaceutically acceptable salt thereof and PEG. Suitably the non-aqueous solution or dispersion comprising niclosamide or a pharmaceutically acceptable salt thereof described herein comprise less than 2% by weight water, preferably less than 0.1% more preferably less that 0.01% by weight water. In particular embodiments the solution or dispersion comprising niclosamide or a pharmaceutically acceptable salt thereof is anhydrous.

Accordingly, it may be that the embodiments it may be that the composition administered to the subject is a non-aqueous solution comprising from about 2% to about 8% by weight niclosamide or a pharmaceutically acceptable salt thereof and from about 92% to about 98% by weight PEG, wherein the PEG has an average molecular weight of from 150 to 600. It may be that the solution is a non-aqueous solution comprising from about 4% to about 8% by weight niclosamide or a pharmaceutically acceptable salt thereof and from about 92% to about 96% by weight PEG, wherein the PEG has an average molecular weight of about 400. It may be that the solution is a non-aqueous solution comprising from about 4% to 8% by weight niclosamide or a pharmaceutically acceptable salt thereof and from about 92% to about 96% by weight PEG, wherein the PEG has an average molecular weight of about 200. It may be that the solution is a non-aqueous solution comprising from about 4% to about 8% by weight niclosamide ethanolamine and from about 92% to about 96% by weight PEG, wherein the PEG has an average molecular weight of about 400. It may be that the solution is a non-aqueous solution comprising from about 4% to 8% by weight niclosamide ethanolamine and from about 92% to about 96% by weight PEG, wherein the PEG has an average molecular weight of about 200.

In certain preferred embodiments the inhalable pharmaceutical composition is a solution or dispersion comprising niclosamide, or a pharmaceutically acceptable salt thereof and is administered to the subject in the form of an aerosol of the solution or dispersion. The solution or suspension comprising niclosamide or a pharmaceutically acceptable salt thereof may be administered to the subject using a suitable nebulizer, inhalation device or intranasal delivery device. For example, it may be that the nebulizer or inhalation device is selected from a vibrating mesh nebulizer, a piezoelectric nebulizer a jet nebulizer and a pressurised metered dose inhaler (pMDI).

In certain embodiments when the inhalable pharmaceutical composition is a liquid (e.g. a liquid solution or suspension comprising niclosamide or a pharmaceutically acceptable salt thereof) the composition is administered as an aerosol of the composition, the aerosol has a mass median diameter of less than about 5 μm. It may be that the MMD of less than about 2 μm. It may be that the MMD of the aerosol is from about 0.5 μm to about 5.5 μm. Preferably the MMD of the aerosol is from about 1 μm to about 5 μm.

Suitably the aerosol of the composition (e.g. aerosol of a solution or suspension comprising niclosamide or a pharmaceutically acceptable salt thereof), has a narrow particle-size distribution, for example a geometric standard deviation (GSD) of less than about 2.2, for example less than 2.0, or less than 1.8. Preferably the GSD of the aerosol is less than 1.6.

In certain embodiments the inhalable pharmaceutical composition is a powder comprising niclosamide, or a pharmaceutically acceptable salt thereof. It may be that the powder comprising niclosamide, or a pharmaceutically acceptable salt thereof is administered to the subject using a dry-powder inhaler. In certain embodiments powder administered to the subject (e.g. particles of niclosamide or a pharmaceutically acceptable salt thereof) have a MMD of less than about 5 μm. For example, the MMD of the powder particles is from about 1 μm to about 5 μm. Suitably the particles administered to the subject (e.g. as an aerosol of the powder) have a GSD of less than about 2.2, for example less than 2.0, or less than 1.8.

Also provided is a method for preventing or treating a viral infection in a subject, the method comprising administering to the subject by inhalation an effective amount of an inhalable pharmaceutical composition comprising niclosamide, or a pharmaceutically acceptable salt thereof. It may be that the inhalable pharmaceutical composition is any of the inhalable compositions disclosed herein. For example, in certain embodiments the inhalable pharmaceutical composition is in the form of a solution comprising niclosamide, or a pharmaceutically acceptable salt thereof; a suspension comprising niclosamide, or a pharmaceutically acceptable salt thereof; an aerosol of a solution comprising niclosamide, or a pharmaceutically acceptable salt thereof; an aerosol of a suspension comprising niclosamide, or a pharmaceutically acceptable salt thereof; or a powder comprising niclosamide or a pharmaceutically acceptable salt thereof.

Also provided is a method for preventing or treating a viral infection is a subject, the method comprising administering to the subject by inhalation an effective amount of a solution comprising niclosamide.

Also provided is a method for preventing or treating a viral infection is a subject, the method comprising administering to the subject by inhalation an effective amount of an aerosol of a solution comprising niclosamide.

Also provided is a method for preventing or treating a viral infection in a subject, the method comprising administering to the subject intranasally an effective amount of a pharmaceutical composition comprising niclosamide, or a pharmaceutically acceptable salt thereof.

Also provided is a method for preventing or treating a viral infection is a subject, the method comprising administering to the subject intranasally an effective amount of a solution comprising niclosamide.

Also provided is a method for preventing or treating a viral infection is a subject, the method comprising administering to the subject intranasally an effective amount of an aerosol of a solution comprising niclosamide.

The solution, suspension, aerosols and powders comprising niclosamide or pharmaceutically acceptable salt thereof used in the methods of treatment may be any of those described herein. For example in certain embodiments the method of treatment administers a solution comprising niclosamide or a pharmaceutically acceptable salt thereof and PEG. Preferably the method of treatment administers an aerosol of a solution comprising niclosamide or a pharmaceutically acceptable salt thereof and PEG. The solution, or aerosol of a solution, comprising niclosamide or a pharmaceutically acceptable salt thereof may be any of the PEG based solutions described herein. For example an aerosol of any of the solutions comprising niclosamide ethanolamine and PEG disclosed herein.

In certain embodiments the niclosamide, or a pharmaceutically acceptable salt thereof, is administered to the subject in a daily dose of from about 1 mg to about 3000 mg based on the weight of niclosamide, for example from about 10 mg to about 3000 mg, based on the weight of niclosamide. Thus it may be that the niclosamide, or a pharmaceutically acceptable salt thereof, is administered to the subject in a daily dose of from about 400 mg to about 2000 mg based on the weight of the niclosamide. Thus it may be that the daily dose is 5 mg, 10 mg, 25 mg, 50 mg, 75 mg, 100 mg, 150 mg, 250 mg, 500 mg, 750 mg, 1000 mg, 1500 mg or 2000 mg, based on the weight of niclosamide. It may be that the total daily dose is administered as a single dose. It may be that the total daily dose is administered as one or more divided doses, for example 2, 3, 4 or 5 divided doses. The total daily dose may be divided evenly or unevenly. Preferably, when the total daily dose is administered as a divided dose the total daily dose is divided into equal doses.

In certain embodiments the niclosamide, or a pharmaceutically acceptable salt thereof, is administered to the subject in a unit dosage of from about 1 mg to about 1000 mg based on the weight of niclosamide, for example from about 10 mg to about 1000 mg based on the weight of niclosamide. Thus it may be that the niclosamide, or a pharmaceutically acceptable salt thereof, is administered to the subject in a unit dosage of from about 100 mg to about 600 mg based on the weight of niclosamide. Preferably the niclosamide, or a pharmaceutically acceptable salt thereof, is administered to the subject in a unit dosage of from about 150 mg to about 500 mg.

In certain embodiments the inhalable pharmaceutical composition comprising niclosamide, or a pharmaceutically acceptable salt thereof, is administered to the subject by inhalation intraorally or intranasally. Preferably the composition is administered to the subject by inhalation intraorally. Thus it may be that the composition, is administered to the subject by inhalation intraorally or intranasally in a form as described herein, for example as a powder, a solution, a suspension, an aerosol of a solution or an aerosol of a suspension comprising the niclosamide or a pharmaceutically acceptable salt thereof as described herein.

In some embodiments the pharmaceutical composition comprising niclosamide, or a pharmaceutically acceptable salt thereof, is administered intranasally. It will be understood that “intranasal” administration means administration into the nasal cavity, i.e. through the nose. Intranasal administration encompasses both administration of the composition to the nasal mucosa and the upper respiratory tract, and administration of the composition to the lower respiratory tract (e.g. via inhalation).

In certain embodiments the inhalable pharmaceutical composition comprising niclosamide, or a pharmaceutically acceptable salt thereof, is administered to the subject by inhalation and/or intranasally at least once per day, for example 1, 2, 3, 4, or 5 times per day. Thus it may be that the composition, is administered to the subject by inhalation and/or intranasally 1 to 4 times per day. It may be that the composition comprising niclosamide, or a pharmaceutically acceptable salt thereof, is administered to the subject by inhalation and/or intranasally once per day. It may be that the composition comprising niclosamide, or a pharmaceutically acceptable salt thereof, is administered to the subject by inhalation and/or intranasally twice per day. It may be that the composition comprising niclosamide, or a pharmaceutically acceptable salt thereof, is administered to the subject by inhalation and/or intranasally three times per day. It may be that the composition comprising niclosamide, or a pharmaceutically acceptable salt thereof, is administered to the subject by inhalation and/or intranasally four times per day.

In certain embodiments the viral infection may be a pulmonary viral infection.

In certain embodiments the viral infection may be caused by or associated with a virus selected from respiratory syncytial virus, influenza virus, parainfluenza virus, human metapneumovirus, severe acute respiratory syndrome coronavirus (SARS-CoV), severe acute respiratory syndrome coronavirus (SARS-CoV-2), Middle East respiratory syndrome coronavirus (MERS-CoV), human coronavirus OC43, Semliki Forest Virus, a human rhinovirus (HRVs) and human adenovirus (HAdV).

In certain embodiments the viral infection is caused by or associated with a Pneumoviridae virus, for example a Human respiratory syncytial virus (HRSV) (e.g. HRSV-A2, HRSV-B1 or HRSV-S2).

In certain embodiments the viral infection is caused by or associated with a Coronaviridae virus. In certain embodiments the viral infection is caused by or associated with a virus is selected from Alphacoronavirus, Betacoronavirus, Gammacoronavirus and Deltacoronavirus. Preferably the viral infection is caused by or associated with a Betacoronavirus. Thus is certain embodiments the viral infection is caused by or associated with a virus is selected from severe acute respiratory syndrome coronavirus (SARS-CoV), severe acute respiratory syndrome coronavirus 2 (SARS-CoV-2), Middle East respiratory syndrome coronavirus (MERS-CoV), HCoV-229E, HCoV-NL63, HCoV-OC43 and HCoV-HKU1.

In a particular embodiment the viral infection is caused by or associated with SARS-CoV-2. This it may be that the viral infection is COVID-19.

In some embodiments the viral infection is caused by or associated with an influenza virus.

The viral infection may be caused by or associated with a virus selected from Flaviviridae (e.g. Zika virus (ZIKV), dengue (e.g. DENV 1-4), West Nile virus (WNV), yellow fever virus (YFV, e.g. yellow fever 17D virus), Japanese encephalitis virus (JEV), Hepatitis C virus (HCV), Filoviridae (e.g. Ebolavirus)), Togaviridae (e.g. Alphaviruses such as Chikungunya virus (CHIKV), Sindbis virus and Ross River virus), Herpes (e.g. γ-herpesvirus, Human herpesvirus 8, herpesvirus 1 and herpesvirus 2) and Adenoviridae (e.g. Human adenoviruses (HAdVs)).

Recent research has identified a gradient of expression levels of the human angiotensin-converting enzyme (ACE)-2, which is targeted by SARS-CoV-2, from the nasal tissues (high expression) and the distal intrapulmonary regions (low expression). This expression pattern was found to be mirrored by a gradient of SARS-CoV-2 infectivity which was high in the nasal epithelium and markedly reduced in the distal lung (bronchioles, alveoli). In light of these findings, it has been suggested that the nasal surfaces may be the dominant initial site of SARS-CoV-2 infection (Hou et al., “SARS-CoV-2 Reverse Genetics Reveals a Variable Infection Gradient in the Respiratory Tract”, Cell, 2020). Intranasal administration may therefore be beneficial to subjects suffering from mild COVID-19, or those in the early stages of disease, prior to progression to the later stages of the disease which are characterised by pulmonary inflammation. In some embodiments, subjects whose symptoms include a loss of taste and/or smell, and/or ocular symptoms (e.g. one or more of conjunctival hyperemia, chemosis, epiphora, or increased secretions) may be treated via intranasal administration of the composition of the invention. Intranasal administration may also be beneficial for treating asymptomatic subjects, for prophylactic treatment of high risk populations as identified herein (e.g. healthcare professionals, or those with underlying conditions), for treating subjects suspected of having contracted SARS-CoV-2, and/or for treating close contacts of a person with COVID-19.

In some embodiments, the inhalable pharmaceutical composition is administered both intranasally and intraorally. Thus it may be that a first pharmaceutical composition of the invention is administered by inhalation intraorally (e.g. as an aerosol) separately, sequentially or simultaneously with a second pharmaceutical composition, wherein the second pharmaceutical composition is administered intranasally (e.g. as spray). It may be that the first and second pharmaceutical compositions are different. It may be that the first and second compositions are the same.

Subjects with pulmonary viral infections may be prone to coughing when drugs are administered by inhalation. This can make administration of the drug difficult and/or may reduce the dose of drug delivered to the airway and lungs. In certain embodiments the subject is administered an antitussive agent prior to or concurrently with the inhaled niclosamide, or a pharmaceutically acceptable salt thereof. Thus it may be that the subject is treated with antitussive agent is selected from codeine, dextromethorphan, hydrocodone, methadone, butorphanol, benzonatate, ethylmorphine, oxeladin, pipazethate, pholcodine, noscapine, butamirate and a local anaesthetic (e.g. lidocaine) prior to or concurrently with the inhaled administration of the niclosamide or a pharmaceutically acceptable salt thereof. Preferably the subject is treated with the antitussive agent prior to administration of the niclosamide or pharmaceutically acceptable salt thereof to reduce or eliminate coughing associated with the inhaled administration of the niclosamide. Thus it may be that the subject is treated with a local anaesthetic prior to or concurrently with the inhaled administration of the niclosamide or a pharmaceutically acceptable salt thereof. Suitably the local anaesthetic is administered so as to provide a local anaesthetic effect in the oral cavity and/or airways. Thus it maybe that the local anaesthetic is administered by inhalation or as a gel or liquid to the oral and/or nasal cavity. Suitably the local anaesthetic is lidocaine.

In some embodiments, the subject is treated with a bronchodilator prior to or concurrently with the composition comprising niclosamide, or pharmaceutically acceptable salt thereof. Advantageously, this may help to reduce side effects, such as coughing. Suitable bronchodilators include short-acting β2-adrenergic agonists (e.g. salbutamol, levosalbutamol, pirbuterol, epinephrine, terbutaline or ephedrine), long-acting β2-adrenergic agonists (e.g. salmeterol, clenbuterol, bambuterol, indacaterol or formoterol), anticholinergics (e.g. tiotropium or ipratropium bromide), and theophylline.

Another aspect provides an aerosol of a solution comprising niclosamide, or a pharmaceutically acceptable salt thereof. Thus it may be that the aerosol is an aerosol of a solution comprising niclosamide, or a pharmaceutically acceptable salt thereof and PEG. The aerosol of a solution, comprising niclosamide or a pharmaceutically acceptable salt thereof may be any of the PEG based solutions of niclosamide described herein. For example, an aerosol of any of the solutions comprising niclosamide ethanolamine and PEG disclosed herein. Suitably the solution is a non-aqueous solution.

Also provided is an inhalable unit dosage comprising a solution of niclosamide, or a pharmaceutically acceptable salt thereof, and PEG, wherein niclosamide is present in an amount of from 1 mg to 600 mg based on the weight of niclosamide, for example from 100 mg to 600 mg based on the weight of niclosamide, for example from about 150 mg to about 500 mg, based on the weight of niclosamide. The solution, comprising niclosamide or a pharmaceutically acceptable salt thereof may be any of the PEG based solutions of niclosamide described herein. For example any of the solutions comprising niclosamide ethanolamine and PEG disclosed herein. Suitably the solution is a non-aqueous solution. The unit dosage is suitably present in a container, for example a vial, blister pack, bottle (e.g. a nasal spray), syringe (e.g. as part of an intranasal delivery device) or reservoir within an inhaler device (e.g. a nebulizer). The unit dosage volume of the solution administered to the subject may be from 1 to 10 ml, from 2 to 9 ml, from 3 to 8 ml or from 4 to 6 ml. In some embodiments, the unit dosage volume administered to the subject is from 10 μl to 10 ml, from 20 μl to 8 ml, from 30 μl to 6 ml, from 40 μl to 5 ml, from 50 μl to 2 ml, from 100 μl to 1 ml, from 120 μl to 0.8 ml, from 130 μl to 0.7 ml, from 140 μl to 0.6 ml, from 150 μl to 0.5 ml or from 200 μl to 400 μl. In some embodiments, the unit dosage volume administered to the subject is from 100 to 200 μl, from 110 to 190 μl, from 120 to 180 μl, from 130 to 170 μl, from 140 to 160 μl or from 150 to 155 μl. It will be appreciated that the mass of the niclosamide, or a pharmaceutically acceptable salt thereof, administered for a given volume will depend on the concentration of the solution. In some embodiments, the niclosamide, or a pharmaceutically acceptable salt thereof, is present in the solution in an amount of from about 0.01% to about 10% by weight. Preferably the solution comprises about 5% by weight of niclosamide ethanolamine. The volume may be administered one or more times per day, for example once per day, twice per day, three times per day or four times per day. It may be that the volume is administered once or twice per day. It may be that the volume is administered once per day. It may be that the volume is administered twice per day.

In some embodiments wherein the solution is administered intranasally, the volume administered to the subject may be from 50 to 500 μl, from 100 to 400 μl, from 150 to 300 μl or from 200 to 250 μl. It will be appreciated that approximately half of the volume should be administered to each nostril. In some embodiments, from about 50 to about 150 μl is administered to each nostril (i.e. about 100 to about 300 μl in total). In some embodiments, a volume of about 130 μl-150 μl (e.g. 140 μl) is administered to each nostril (i.e. about 260-300 μl, e.g. 280 μl, in total). Preferably the solution administered intranasally comprises about 5% by weight of niclosamide ethanolamine. The volume may be administered intranasally one or more times per day, for example once per day, twice per day, three times per day or four times per day. It may be that the volume is administered intranasally once or twice per day. It may be that the volume is administered intranasally once per day. It may be that the volume is administered intranasally twice per day. It will further be appreciated that in some embodiments wherein the solution is administered both intraorally and intranasally, the total volume administered to the subject will be the sum of the volume administered intraorally and the volume administered intranasally. The total volume may be from 10 μl to 10 ml, from 20 μl to 8 ml, from 30 μl to 6 ml, from 40 μl to 5 ml, from 50 μl to 2 ml, from 100 μl to 1 ml, from 150 μl to 0.5 ml or from 200 μl to 400 μl. Preferably the solution administered intranasally comprises about 5% by weight of niclosamide ethanolamine. As will be appreciated, when the solution is administered both intraorally and intranasally the volume administered intranasally may be the same or different to the volume administered intranasally. Similarly the frequency of the intraoral and intranasal administration may be the same or different. For example, the intraoral and intranasal doses may be administered sequentially (e.g. the intraoral administration followed shortly (e.g. within 10 minutes) by the intranasal administration, or vice versa. In certain embodiments the intraoral and intranasal doses may be administered separately (e.g. where the intraoral dosing is separated from the intranasal dosing by more than 10 minutes (e.g. by more than one hour). Also contemplated is substantially simultaneous intraoral and intranasal administration. It may be that the volume administered intranasally is administered once or twice per day. It may be that the volume administered intraorally is administered once or twice per day.

Also provided is a system comprising a container comprising an inhalable pharmaceutical composition comprising niclosamide, or a pharmaceutically acceptable salt thereof; and an inhaler device.

Also provided is a kit comprising a container comprising an inhalable pharmaceutical composition comprising niclosamide, or a pharmaceutically acceptable salt thereof; and an inhaler device.

Also provided is a system comprising a container comprising: a pharmaceutical composition comprising niclosamide, or a pharmaceutically acceptable salt thereof; and an intranasal delivery device.

Also provided is a kit comprising a container comprising a pharmaceutical composition comprising niclosamide, or a pharmaceutically acceptable salt thereof; and an intranasal delivery device.

In certain embodiments the inhalable pharmaceutical composition comprising niclosamide, or a pharmaceutically acceptable salt thereof present in the system or kit is in the form of a powder, a solution, a suspension, for example any of the niclosamide compositions described herein. Suitably the inhalable pharmaceutical composition is a non-aqueous composition.

In certain embodiments the inhalable pharmaceutical composition comprising niclosamide, or a pharmaceutically acceptable salt thereof present in the system or kit is a solution comprising niclosamide, or a pharmaceutically acceptable salt thereof and PEG. For example any of the PEG-based niclosamide solutions described herein. For example, any of the solutions comprising niclosamide ethanolamine and PEG disclosed herein.

In certain embodiments the inhaler device of the system or kit is adapted to aerosolize a solution or suspension comprising niclosamide or a pharmaceutically acceptable salt thereof. Suitably the inhaler device is adapted to deliver the aerosolized solution or suspension intranasally or intraorally to a subject.

In certain embodiments the inhaler device of the system or kit is a nebulizer selected from a jet nebulizer, a vibrating mesh nebulizer, an ultrasonic nebulizer or a pressurised metered dose inhaler (pMDI).

Also provided is an inhalable solution comprising from about 1% to 10% by weight niclosamide ethanolamine and PEG, wherein the PEG has an average molecular weight of less than 600; and optionally a taste-masking agent. Thus it may be the inhalable solution is any of the solutions described herein comprising niclosamide or a pharmaceutically acceptable salt thereof and PEG, for example any of the solutions disclosed herein comprising niclosamide ethanolamine and PEG. Thus it may be the inhalable solution comprises from about 1% to 10% by weight niclosamide ethanolamine and PEG with an average molecular weight of 400. Thus it may be the inhalable solution comprising from about 1% to 10% by weight niclosamide ethanolamine and PEG with an average molecular weight of 200. In certain embodiments the inhalable solution is a solution comprising from about 4.5% to about 6.5% by weight niclosamide ethanolamine and about 93.5% to about 95.5% by weight PEG 400. In certain embodiments the inhalable solution comprises from about 4.5% to about 6% by weight niclosamide ethanolamine; about 90% to 95.5% by weight PEG 400 and optionally a taste-masking agent. In certain embodiments the inhalable solution comprises from about 4.5% to about 6% by weight niclosamide ethanolamine; about 90% to 95.5% by weight PEG 200 and optionally a taste-masking agent.

Embodiments of the invention will now be described by way of example and with reference to the accompanying Figures, in which:

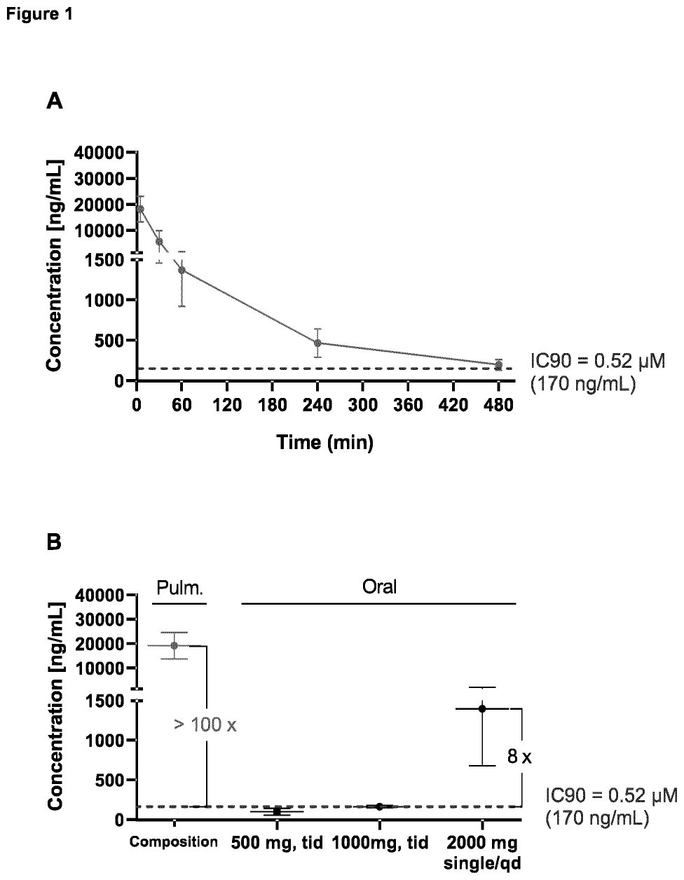

FIG. 1 shows graphs showing the epithelial lining fluid (ELF) concentration of niclosamide free base following pulmonary administration in sheep compared to systemic exposure of highest human oral dose, relative to IC90 against SARS-CoV-2. (A) Mean ELF concentration of niclosamide over time following pulmonary administration of a composition of the invention (±SEM); (B) Comparison of mean Cmax levels of niclosamide following administration of a composition of the invention in ELF to systemic Cmax following a 2 g/day oral dose in humans (Data of Andrews et al. 1983, Pharmacology & therapeutics, 19(2), 245-295 (healthy volunteers) and Burock et al. 2018, BMC Cancer, 18(1): 297 (colorectal cancer patients) combined in “2000 mg single/qd” column);

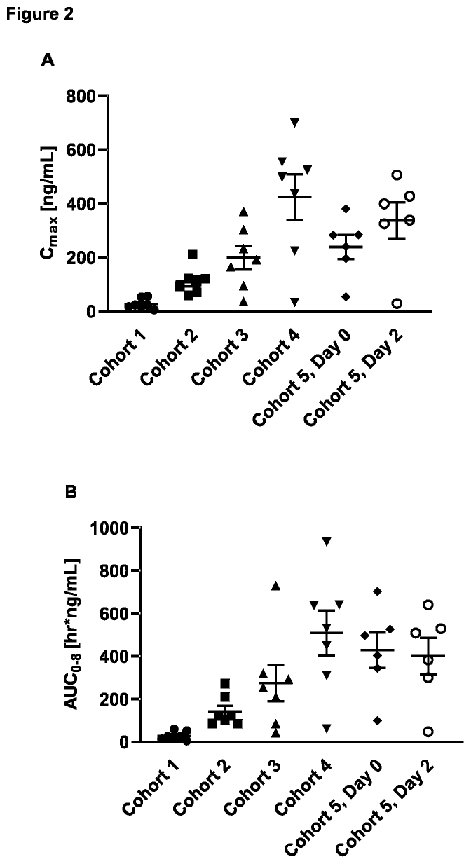

FIG. 2 shows plots showing the pharmacokinetic profile of niclosamide ethanolamine per cohort in the phase 1 clinical trial described in Example 8;

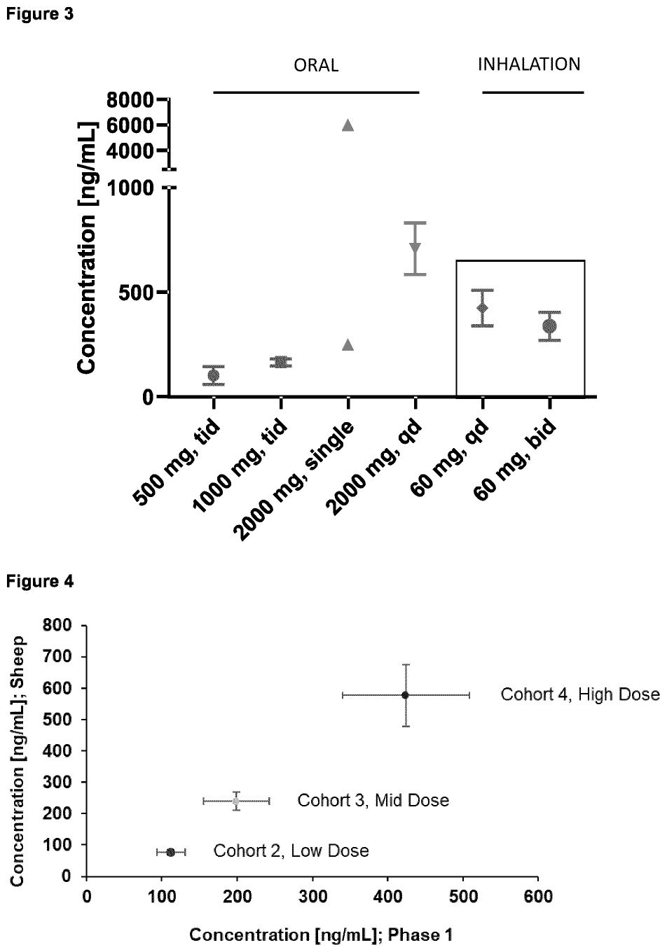

FIG. 3 is a comparison of systemic exposure (Cmax; mean±SEM) of niclosamide administered orally (as reported in the literature) versus inhalation of a composition according to the invention in humans. No mean for “2000 mg, single” column generated as only range of Cmax reported in literature. Data for 500-1000 mg obtained from Schweizer et al., 2018, PLoS ONE.; 13(6): e0198389. Data for 2000 mg obtained from Andrews et al. 1983 and Burock et al. 2018 (as above);

FIG. 4 shows a correlation plot of systemic exposure (Cmax-, mean±) of human versus sheep study;

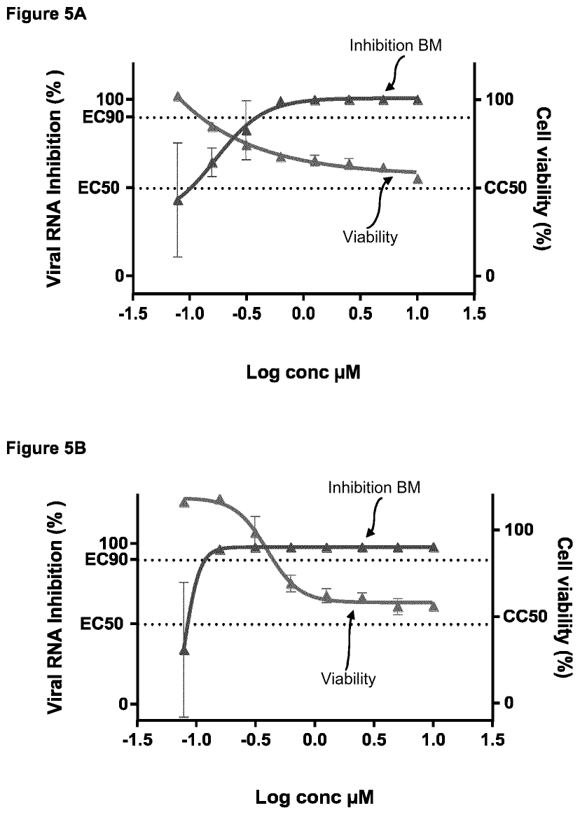

FIG. 5 shows the Inhibition of SARS-CoV-2 replication in VeroE6 cells (FIG. 5A) and Caco-2 cells (FIG. 5B) by niclosamide ethanolamine salt;

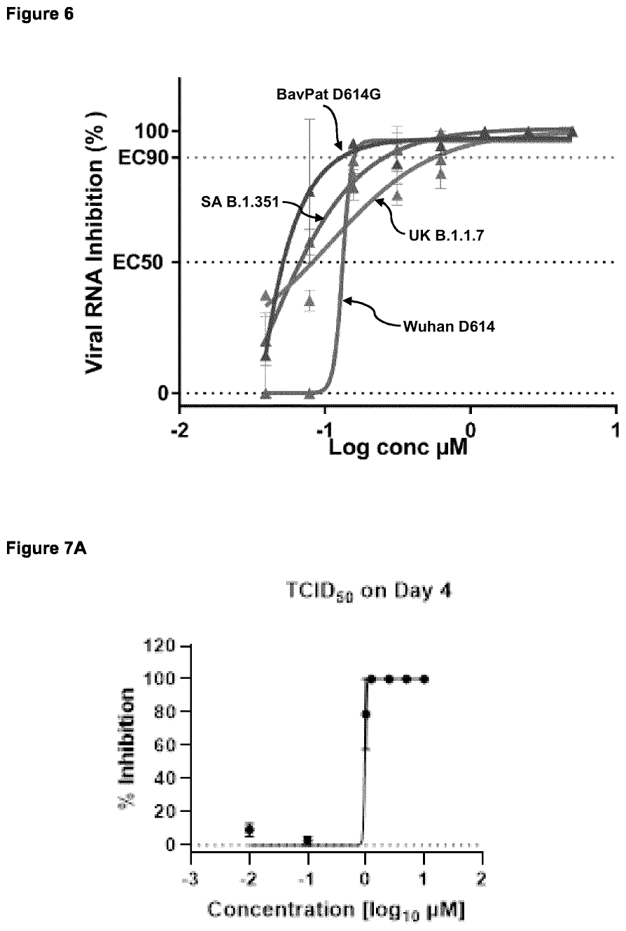

FIG. 6 is a graph showing that niclosamide ethanolamine salt inhibits replication of several variants of SARS-CoV-2;

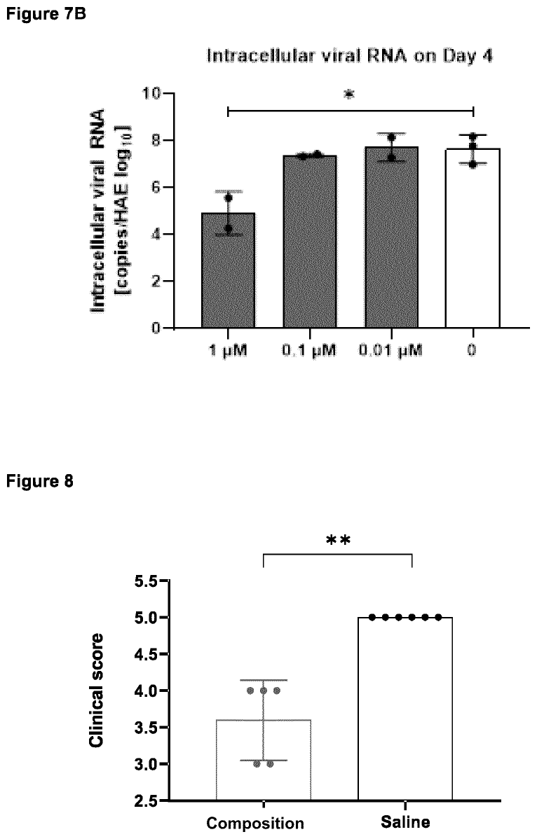

FIG. 7 shows the effect of niclosamide ethanolamine salt on apical viral infectious titer TCID50 (FIG. 7A) and intracellular RNA levels (FIG. 7B) of SARS-CoV-2 in a trans-well system of infection. N=2. Mean with 95% levels shown for FIG. 7A, and Mean±SD for FIG. 7B. * p<0.05, Ordinary one-way ANOVA with Dunetts' multiple comparison test; and

FIG. 8 shows the clinical score of SARS-COV-2 infected K18-hACE2 transgenic mice on Day 6 post infection with a composition according to the invention compared to saline. N=5 for composition and N=6 for saline. **=p<0.01 (Mann Whitney Test).

### DETAILED DESCRIPTION

### Definitions

Unless otherwise stated, the following terms used in the specification and claims have the following meanings set out below.

References to “composition of the invention”, “compositions of the invention” “solution of the invention” refer to any of the compositions described herein comprising niclosamide, or a pharmaceutically acceptable salt thereof. A “solution of the invention” refers to a composition of the invention wherein the niclosamide or pharmaceutically acceptable salt thereof is dissolved in the composition. The terms “composition” and “formulation” may be used interchangeably.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the subject; slowing in the rate of degeneration or decline; making the final point of degeneration less debilitating; improving the physical or mental well-being of the subject. For example, in relation to the treatment of viral infections disclosed herein the treatment may include one or more of the following: Reduce or eliminate the virus; prevent or reduce viral replication; reduce or eliminate transmission of the virus; reduce or eliminate fever; reduce or eliminate flu-like symptoms, reduce or eliminate coughing, reduce or eliminate muscle and/or joint pain; improve respiratory status of the subject (e.g. increasing blood oxygen saturation; reducing or eliminating the requirement for oxygen therapy); an improvement in the NEWS2 score; the prevention or treatment of acute respiratory distress syndrome associated with the viral infection; the treatment or prevention of pneumonia associated with the viral infection; the treatment or prevention of viral pneumonia; the treatment or prevention of bacterial pneumonia associated with a viral infection; reducing or eliminating pulmonary edema; reducing or eliminating pulmonary inflammation; preventing or reducing lung fibrosis (e.g. preventing or reducing interstitial fibroblasts); reducing one or more inflammatory biomarkers associated with the viral infection (e.g. reducing one or more of CRP, leukocytes, IL1B, IL-6, IL-10, IL-2, IFNγ, IP10, MCP1, GCSF, IP10, MCP1, MIP1A, and/or TNFα, particularly reducing serum CRP); preventing or reducing proteinaceous exudates associated with a viral infection; preventing or reducing fibrin exudates associated with a viral infection; and/or preventing or ameliorating pulmonary bacterial or fungal infections associated with the viral infection. Also contemplated are prophylactic treatments, wherein a subject is treated with an inhaled composition of the invention to prevent or reduce the risk of a subject contracting a disease (e.g. viral infection) or to prevent a disease or condition from becoming symptomatic.

The term “associated” or “associated with” in the context of a substance or substance activity or function associated with a disease (e.g. a viral infection such as SARS-CoV-2) means that the disease is caused by (in whole or in part), or a symptom of the disease is caused by (in whole or in part) the substance or substance activity or function.

When a compound or salt (e.g. niclosamide or a pharmaceutically acceptable salt thereof) described in this specification is administered to treat a disorder, a “therapeutically effective amount” is an amount sufficient to reduce or completely alleviate symptoms or other detrimental effects of the disorder; cure the disorder; reverse, completely stop, or slow the progress of the disorder; or reduce the risk of the disorder getting worse.

The term “pharmaceutically acceptable salt” refers to salts that retain the biological effectiveness and properties of the compounds described herein and, which are not biologically or otherwise undesirable. Pharmaceutically acceptable salts of niclosamide are well known to skilled persons in the art. Particular niclosamide salts include ethanolamine or piperazine salts. Accordingly, it may be that a reference to a salt of niclosamide herein may refer to a pharmaceutically acceptable salt of niclosamide, in particular an ethanolamine salt of niclosamide (e.g. the 1:1 salt of niclosamide with 2-aminoethanol).

The term “solvate” is used herein to refer to a complex of solute, such as a compound or salt of the compound, and a solvent. If the solvent is water, the solvate may be termed a hydrate, for example a monohydrate, dihydrate, trihydrate etc., depending on the number of water molecules present per molecule of substrate. Reference to “niclosamide, or a pharmaceutically acceptable salt or hydrate thereof” includes hydrates of niclosamide and hydrates of a salt of niclosamide. Suitably the niclosamide or pharmaceutically acceptable salt thereof is anhydrous form.

Unless stated otherwise references to “inhalable pharmaceutical composition”, “inhalable pharmaceutical composition of the invention”, “inhaled composition”, “inhalable composition”, “a composition comprising niclosamide” or “a composition of the invention” are also applicable to pharmaceutical compositions of the invention which may be administered intranasally. Thus, references to “inhalable pharmaceutical composition”, “inhalable pharmaceutical composition of the invention”, “inhaled composition”, “inhalable composition”, “a composition comprising niclosamide” or “a composition of the invention” refer to an inhalable pharmaceutical composition comprising niclosamide, or a pharmaceutically acceptable salt thereof administered to the subject by inhalation, and also to a pharmaceutical composition comprising niclosamide, or a pharmaceutically acceptable salt thereof administered to the subject intranasally. For example, any of the inhalable compositions described herein including, but to limited to a powder, a solution, a suspension, an aerosol of a solution or an aerosol of a suspension comprising niclosamide or a pharmaceutically acceptable salt thereof, may additionally or alternatively be administered intranasally. For the avoidance of doubt, it will be understood that all aspects and embodiments described above, including compositions, unit dosages, dosage regimes, volumes, amounts of niclosamide or PEG by % weight, devices, particle sizes and methods of treatment, are equally applicable to both an inhalable pharmaceutical composition, and a pharmaceutical composition which is administered intranasally.

Reference to “PEG x00” herein means a polyethylene glycol with an average molecular weight of x00. For example, PEG 400 refers to a PEG with an average molecular weight of 400. Unless stated otherwise reference herein to the molecular weight of polymer, such as a PEG is a reference to number average molecular weight (Mn) of the polymer. The number average molecular weight can be measured using well known methods, for example by gel permeation chromatography or 1H NMR end-group analysis. Such methods include GPC analysis as described in Guadalupe et al (Handbook of Polymer Synthesis, Characterization, and Processing, First Edition, 2013) and end group analysis described in e.g. Page et al Anal. Chem., 1964, 36 (10), pp 1981-1985.

Reference to an “aerosol” means the suspension of solid particles or liquid droplets comprising niclosamide or a pharmaceutically acceptable salt thereof in a gas (e.g. air or a suitable propellant gas). An aerosol comprising liquid droplets comprising liquid droplets is suitably formed by aerosolizing a solution or suspension comprising the niclosamide or a pharmaceutically acceptable salt thereof, for example any of the solutions or suspensions described herein. The continuous gas phase of the aerosol may be selected from any gas or mixture of gases which is pharmaceutically acceptable. Preferably the gas may simply be air or compressed air. Alternatively, other gases and gas mixtures, such as air enriched with oxygen, carbon dioxide, or mixtures of nitrogen and oxygen may be used. Aerosolization may be achieved using a suitable inhalation device, for example a nebulizer described herein.

The particle/droplet size of an aerosol may be measured as the mass median diameter (MMD) of the aerosol droplet/particles. The MMD may be measured using well-known methods, for example a laser diffraction technique using a Malvern MasterSizer X™. Suitably, the MMD may be determined by nebulizing a suitable volume of the solution or suspension (e.g.) 2 mL using a suitable nebulizer device. The resulting aerosol is analysed by directing by directing the aerosol cloud through the laser beam of the MasterSizer X™ instrument using an aspiration flow of 20 L/min at a temperature of 23° C. (±2° C.) and a relative humidity of 50% (±5%).

The Geometric Standard Deviation (GSD) is a measure of the measure the particle or droplet size distribution in an aerosol. The GSD may be determined using known methods, for example using well-known laser diffraction methods, for example using a MasterSizer X™ under the same conditions described above for the measurement of MMD.

Reference to a “subject” herein means a human or animal subject. Preferably the subject is warm-blooded mammal. More preferably the subject is a human.

Unless stated otherwise, reference herein to a “% by weight of niclosamide, or a pharmaceutically acceptable salt thereof” is intended to refer to the amount of the free acid (i.e. non-salt form) of the niclosamide. For example, reference to a composition comprising “5% by weight of niclosamide or a pharmaceutically acceptable salt thereof” refers to a composition comprising 5% by weight of niclosamide as the free acid. Accordingly, where such a composition comprises a pharmaceutically acceptable salt of niclosamide, the absolute amount of the salt niclosamide in the composition will be higher than 5% by weight in view of the salt counter ion that will be also be present in the composition.

Reference to a “non-aqueous” composition, means that the composition is anhydrous and therefore substantially water free. For example, the compositions disclosed herein (e.g. solutions or suspensions comprising niclosamide or a pharmaceutically acceptable salt thereof) contain less than 5%, less than 1% or suitably less than 0.01%, preferably less than 0.001% by weight water. Preferred non-aqueous compositions are those which are anhydrous and contain no detectable water.

As will be recognised by the skilled person, reference to administering by inhalation a solution or suspension comprising niclosamide or a pharmaceutically acceptable salt thereof requires the solution or suspension to be delivered to the subject in a form that is suitable for inhalation. Typically the solution or suspension will be delivered in the form of a spray or, preferably in the form of an aerosol formed from the solution or suspension. Methods and devices for delivering a liquid or suspension in an inhalable form are well known and include nebulizers and pMDI inhalers.

Where reference is made herein to an inhaled composition of the invention for use in in the treatment of a condition (e.g. a viral infection) is to be under stood as also encompassing a method for the treatment of that condition in a subject by administering an effective amount of the composition to the subject; and use of the subject for the manufacture of a medicament for the treatment of the condition.

Reference to “about” in the context of a numerical is intended to encompass the value+/−10%. For example, about 20% includes the range of from 18% to 22%.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Niclosamide

Disclosed herein is the treatment of viral infections comprising the inhalation of a pharmaceutical composition comprising niclosamide, or a pharmaceutically acceptable salt thereof.

The structure of niclosamide is shown below:

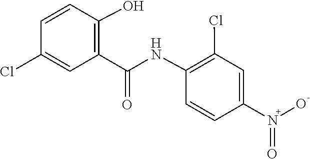

### Niclosamide: 5-chloro-N-(2-chloro-4-nitrophenyl)-2-hydroxybenzamide

In certain embodiments, the niclosamide is in the free acid form in the composition. In certain embodiments the niclosamide is in the form of a pharmaceutically acceptable salt of niclosamide (or a solution thereof), for example an ethanolamine salt, or piperazine salt. A preferred pharmaceutically acceptable salt of niclosamide in the inhalable composition is niclosamide ethanolamine.

In some embodiments the niclosamide may be present in the composition as a hydrate of niclosamide or pharmaceutically acceptable salt thereof is used. However, generally it is preferred that the niclosamide is not in the form of a hydrate. Thus, in certain embodiments the inhalable composition comprises anhydrous niclosamide, or a pharmaceutically acceptable salt thereof. For example, it may be that the niclosamide is anhydrous niclosamide. It may be that the niclosamide is anhydrous niclosamide ethanolamine.

### Inhalable Pharmaceutical Composition Comprising Niclosamide

The niclosamide or pharmaceutically acceptable salt thereof may be present in any pharmaceutical composition suitable for administration by inhalation. Preferred inhalable compositions comprising niclosamide or a pharmaceutically acceptable salt thereof include for example, compositions in the form of a solution, suspension, powder, an aerosol of a solution or an aerosol of a suspension as described in more detail herein.

Also contemplated are other inhalable compositions comprising niclosamide or a pharmaceutically acceptable salt thereof, for example: solid lipid particles comprising niclosamide dissolved or dispersed therein; emulsions comprising niclosamide or a pharmaceutically acceptable salt thereof (e.g. an oil-in water emulsion wherein niclosamide or a pharmaceutically acceptable salt thereof, is dissolved or dispersed in the oil-phase of the emulsion); or liposomes comprising the niclosamide or a pharmaceutically acceptable salt thereof.

### Solutions and Suspensions Comprising Niclosamide

In certain embodiments the inhalable pharmaceutical composition comprising niclosamide is an inhalable solution or suspension comprising niclosamide or a pharmaceutically acceptable salt thereof. Preferably the solution or suspension is a liquid, more preferably a liquid that is suitable for aerosolization using for example a nebulizer inhaler. Thus a reference herein to any of the solutions or suspensions comprising niclosamide or a pharmaceutically acceptable salt thereof are preferably liquid solutions or liquid suspensions comprising the niclosamide or pharmaceutically acceptable salt thereof.

In certain embodiments the niclosamide or a pharmaceutically acceptable salt thereof is dissolved or dispersed in a liquid medium to provide a solution or suspension suitable for inhalation. In certain embodiments the niclosamide or a pharmaceutically acceptable salt thereof is dissolved or dispersed in a medium selected from a non-polymeric glycol (for example an alkylene glycol, e.g. a C₂₋₈ alkylene glycol such as propylene glycol); a polymeric glycol (for example a poly(alkylene glycol), e.g. a polyethylene glycol or a polypropylene glycol); a glycol ether (e.g. 2-(2-ethoxyethoxy)ethanol (Transcutol)); glycerol; an oil (e.g. a non polar oil); or a hydrocarbon solvent. In certain embodiments the niclosamide or a pharmaceutically acceptable salt thereof is dissolved or suspended in an oil. For example, it certain embodiments the niclosamide or a pharmaceutically acceptable salt thereof is dissolved or suspended in a mineral oil, a vegetable oil and long-chain or medium chain triglycerides. It may be that the solution or suspension comprises an aqueous solution or suspension comprising niclosamide or a pharmaceutically acceptable salt thereof. However, it is preferred that the solution or dispersion is a non-aqueous solution or suspension comprising niclosamide or a pharmaceutically acceptable salt thereof.

It may be that the solution or suspension described herein further comprises one or more surfactant or emulsifier, particularly when the niclosamide is present as a dispersion in the liquid medium. It may be that the solution or dispersion further comprises one or more ionic or non-ionic surfactant or emulsifier. Representative examples of surfactants or emulsifiers include any of those described herein, for example a PEGylated fatty acid glyceride (labrasol), polyoxyethylene glycol sorbitan alkyl ester (polysorbate, e.g Tween 20 or Tween 80), a polyoxyethylene glycol alkyl ether (Brij), polyoxyethylene ethers of fatty alcohols (ceteareth), a fatty acid ester of glycerol (e.g. glyceryl stearate) a protein emulsifier (e.g. albumin). It may be that the surfactant or emulsifiers are present in the solution or dispersion in an amount of from about 0.1% to about 15%, about 0.2% to about 10%, or about 0.2% to about 5% by weight of the composition.

In embodiments where the niclosamide or pharmaceutically acceptable salt thereof is present as a suspension in a liquid medium, the niclosamide may be present in the liquid medium as a microparticle suspension or a nanosuspension. Microparticle suspensions may be prepared by milling the niclosamide or a pharmaceutically acceptable salt thereof to provide an average particle size of less than 5 μm, preferably less than 2 μm. If required the particle size of the niclosamide may be reduced using other methods, for example by high pressure homogenization, typically in the presence of a suitable surfactant or emulsifier (for example a surfactant or emulsifier described herein).

Nanosuspensions comprising niclosamide in the liquid medium may be prepared using well-known methods, for example by nano-precipitation, high-pressure homogenization or through spray drying a solution of niclosamide. Typically nanosuspensions comprising niclosamide further comprise a stabilizer, for example a surfactant or emulsifier to maintain the nanoparticles in suspension. The nanoparticles comprising the nanosuspension suitably have an average particle size of less than about 1000 nm, more preferably less than about 400 nm, less than about 300 nm, less than about 250 nm, or less than about 200 nm, as measured by light-scattering methods.

The niclosamide may be present in the solution or suspension in the liquid medium in any of the amounts described herein. When the niclosamide, or pharmaceutically acceptable salt thereof is present as a solution, the solution typically contains from about 0.5 to 10% by weight of the niclosamide or pharmaceutically acceptable salt thereof. When the niclosamide is present as a suspension in a liquid medium higher amounts of the niclosamide or pharmaceutically acceptable salt thereof may be present, for example up to 12%, 15%, 18%, 20%, 22% or 25% by weight of the composition. Also contemplated are suspensions wherein some of the niclosamide or pharmaceutically acceptable salt thereof is dissolved in the liquid medium and some is dispersed in the liquid medium.

### Suspensions or Solutions Comprising PEG

In certain embodiments the solution or suspension comprises niclosamide or pharmaceutically acceptable salt and PEG. Thus it may be that the inhalable pharmaceutical composition is a liquid suspension comprising niclosamide or pharmaceutically acceptable salt and PEG. Preferably the inhalable pharmaceutical composition is a solution comprising niclosamide or pharmaceutically acceptable salt and PEG.

Suitably the PEG is liquid at ambient temperature (for example 20 to 25° C.), accordingly the solvent may be a low molecular weight PEG. Particularly, the PEG has an average molecular weight of 600 or less, suitably less than 600. For example, the PEG may have an average molecular weight of from about 200 to about 600, about 200 to about 500 or about 200 to about 400. A particular PEG is selected from PEG 200, PEG 300 and PEG 400. In certain embodiments the PEG is PEG 200. Preferably the PEG is PEG 400.

Suitably the PEG is present in the solution or dispersion in an amount of greater than 60%, 65%, 70%, 75%, 80%, 85%, 90%, 91%, 92%, 93%, 94%, 95%, 96%, 97% or 98%, wherein the % is by weight based upon the weight of the solution or dispersion. Thus it may be that the PEG is present from 60% to 99%, from 60% to about 98%, from 65% to about 98%, from about 70% to about 98%, from about 75% to about 98% from about 80% to about 98%, from about 85% to about 98%, from about 75% to about 96%, from about 80% to about 96%, from about 85% to about 96%, from about 90% to about 96%, from about 91% to about 96%, from about 92% to about 96% or from about 93% to about 96%, wherein all % are by weight of the solution or dispersion. It may be that the PEG is present in an amount of about 90%, about 91% about 92%, about 93%, about 94%, about 95%, about 96% about 97%, or about 98% or about 99% by weight of the solution or dispersion.

When the niclosamide or pharmaceutically acceptable salt thereof is administered to the subject as a solution comprising PEG, the solution suitably comprises niclosamide, or a pharmaceutically acceptable salt thereof in an amount of about 0.01% to about 10% by weight of the solution. For example, the niclosamide or a pharmaceutically acceptable salt thereof is present in an amount of 0.05% to 10%, 0.05% to 8%, 0.5% to 8%, 1% to 8%, 1.5% to 8%, 2% to 8%, 2.5% to 8%, 3% to 8%, 3.5% to 8%, 4% to 8%, 4.5% to 8%, 5% to 8%, 5.5% to 8%, 6% to 8%, 3% to 7%, 3.5% to 7.5%, 3.5% to 7%, 3.5% to 6.5%, 3.5% to 6%, 3.5% to 5.5%, 4% to 7%, 4% to 7%, 4% to 6.5%, 4% to 6%, 4% to 5.5%, 4.5% to 7%, 4.5% to 6.5%, 4.5% to 6.5% or 4.5% to 5.5% by weight of the solution. In a preferred embodiment the niclosamide or a pharmaceutically acceptable salt thereof is present in the composition in an amount of about 4.5% to 5.5% by weight of the solution. Thus it may be that the niclosamide or a pharmaceutically acceptable salt is present in the composition in an amount of about 3.5% about 4%, about 4.5%, about 5%, about 5.5%, about 6%, about 6.5%, about 7%, about 7.5%, or about, 8% by weight of the solution. In a preferred embodiment the composition comprises about 5% by weight of niclosamide or a pharmaceutically acceptable salt thereof.

In some embodiments the solution or dispersion comprising niclosamide or a pharmaceutically acceptable salt thereof and PEG further comprises water, for example up to 30% by weight water. Suitably however, the solutions or dispersions are non-aqueous solutions or dispersions.

In certain embodiments the solution is a non-aqueous solution comprising from about 1% to about 10% by weight niclosamide ethanolamine and PEG, wherein the PEG has an average molecular weight of less than 600. Thus it may be that the inhalable solution is a non-aqueous solution comprising from about 4.5% to about 6% by weight niclosamide ethanolamine; and about 90% to 95.5% PEG 400. In a particular embodiment the solution is a non-aqueous solution comprising about 5% by weight niclosamide ethanolamine and at least 90% PEG 400.

In certain embodiments the solutions or suspensions comprising niclosamide described herein further comprise one or more solvents in addition to the liquid medium. For example the solution or dispersion may further comprise an organic solvent, for example a polar organic solvent. Thus it may be that the solution or suspension further comprises one of more organic solvents selected from: propylene glycol, glycerol, 2-(2-ethoxyethoxy)ethanol (Transcutol), propylene glycol stearyl ether and propylene glycol isostearate. The additional organic solvent is optionally present in an amount of up to about 30% by weight of the solution or suspension, for example from about 1% to about 25%, from about 1% to about 20%, or from about 1% to about 10% by weight of the solution or suspension. In certain embodiments the solution or dispersion does not comprise an solvents other than PEG.

In certain embodiments the inhalable pharmaceutical composition (e.g. inhalable solution or suspension) does not comprise volatile organic solvents. Thus in certain embodiments the composition of the invention does not contain volatile alcohols, for example methanol ethanol, propanol or isopropanol.

In certain embodiments the solution or suspension comprising niclosamide or a pharmaceutically acceptable salt thereof has a dynamic viscosity of from about 1 to about 150 mPa·s (at 20° C.).

### Aerosols of Solutions and Suspensions Comprising Niclosamide

The solution or suspension comprising niclosamide or a pharmaceutically acceptable salt thereof is administered to the subject in a form suitable for inhalation. For example the solution or suspension may be administered as a spray, preferably as an aerosol of the solution or suspension comprising niclosamide or a pharmaceutically acceptable salt thereof.

Aerosols of the solutions and dispersions comprising niclosamide or a pharmaceutically acceptable salt thereof disclosed herein form a further aspect of the invention.

Inhalation of the inhalable composition of the invention (e.g. an aerosol of a solution or dispersion comprising niclosamide or a pharmaceutically acceptable salt thereof) delivers the niclosamide or pharmaceutically acceptable salt thereof to the airways of the subject. In certain embodiments inhalation of the aerosol delivers niclosamide to the upper respiratory tract for example one or more of the nose and nasal passages, paranasal sinuses, the pharynx, the portion of the larynx above the vocal cords. Preferably, inhalation of the aerosol delivers niclosamide to the lower respiratory tract, for example one or more of the trachea, lungs, bronchi, bronchioles, alveolar duct or alveoli.

In certain embodiments the aerosol of the solution or suspension comprising niclosamide or a pharmaceutically acceptable salt thereof, has a mass median diameter of less than about 5 μm. It may be that the MMD of less than about 2 μm. It may be that the MMD of the aerosol is from about 0.5 μm to about 5.5 μm. Preferably the MMD of the aerosol is from about 1 μm to about 5 μm. Suitably the aerosol has a geometric standard deviation (GSD) of less than about 2.2, for example less than 2.0, or less than 1.8. Preferably the GSD of the aerosol is less than 1.6.

In some embodiments the aerosol of the solution or suspension comprising niclosamide or a pharmaceutically acceptable salt thereof, has a mass median diameter of less than about 500 μm, less than about 300 μm, less than about 250 μm, less than about 200 μm, less than about 150 μm, less than about 100 μm, less than about 90 μm, less than about 80 μm, less than about 70 μm, less than about 60 μm or less than about 50 μm. In some embodiments the MMD of the aerosol is from about 5 to about 150 μm, from about 10 μm to about 120 μm, from about 20 to about 100 μm, from about 30 μm to about 90 μm, from about 40 μm to about 80 μm, or from about 50 μm to about 70 μm, e.g. about 65 μm.

As is known in the art, droplet or particle size distribution may also be defined by reference to D10 and D90 values. 10% of particles or droplets are smaller than the D10 value. 90% of particles or droplets are smaller than the D90 value. In some embodiments, an aerosol of a formulation of the invention has a D10 of from 1 to 200 μm, from 5 to 100 μm, from 10 to 70 μm, from 15 to 50 μm or from 20 to 40 μm (e.g. about 30 μm). In some embodiments, an aerosol of a formulation of the invention has a D90 of from 50 to 500 μm, from 80 to 400 μm, from 100 to 300 μm or from 150 to 250 μm. The particle size distribution may be measured using well-known methods, for example by laser diffraction such as Low-Angle Laser Light Scattering (LALLS) using a SprayTec apparatus from Malvern.

Aerosols of a solution or suspension comprising niclosamide or a pharmaceutically acceptable salt thereof may be formed using known methods, for example via a suitable inhaler device, particularly nebulizers as described herein.

### Powders Comprising Niclosamide

In certain embodiments the inhalable composition is a powder comprising niclosamide, or a pharmaceutically acceptable salt thereof.

Suitably the powder comprises particles comprising niclosamide or a pharmaceutically acceptable salt thereof that are of a respirable size. In certain embodiments the powder has an particle size (MMD) of less than 10 μm, for example less than 5 μm. For example, the MMD of the powder particles is from about 1 μm to about 5 μm. Suitably the particles administered to the subject (e.g. as an aerosol of the powder) have a GSD of less than about 2.2, for example less than 2.0, or less than 1.8.

Powders suitable for inhalation may be prepared using well-known methods, for example by grinding or milling niclosamide or a pharmaceutically acceptable salt thereof, or a composition comprising the compound. Respirable powders may also be formed by, for example micro-precipitation, lyophilisation or spray drying, or spray-freeze drying a solution comprising niclosamide or a pharmaceutically acceptable salt thereof.

In certain embodiments respirable particles comprising niclosamide or a pharmaceutically acceptable salt thereof may be prepared by precipitation, lyophilisation or spray drying, or spray-freeze drying a solution comprising the niclosamide and a suitable carrier to provide respirable powder particles comprising the niclosamide and the carries as composite particles. Suitable carriers include inert carriers such as starch, sugars (e.g. mannitol, lactose or trehalose).

In certain embodiments powders comprising respirable particles of niclosamide or a pharmaceutically acceptable salt thereof may be formulated with carrier particles. It may be that the carrier particles a larger than the particles of niclosamide and the mixing of the carrier with the respirable niclosamide powder forms an “ordered mixture”. Such ordered mixtures can be useful in dry powder inhalers. The fine particles of niclosamide powder loosely associate with the larger carrier particles (e.g. approximately 100 μm) to facilitate the filling and storage of the powder in an inhaler reservoir of unit dosage (e.g. vial, capsule or blister pack). Upon administration from the inhaler the turbulence and/or mechanical impaction experienced by the powder releases the fine particles of drug from the larger carrier particles to provide a respirable fine particle fraction of drug which is inhaled into the respiratory tract of the subject. Carriers suitable for the preparation of ordered mixtures include, for example lactose, mannitol and microcrystalline cellulose.

Powders comprising niclosamide or a pharmaceutically acceptable salt thereof may be administered to the subject using a suitable dry powder inhaler.

### Other Components in Inhalable Pharmaceutical Compositions

The inhalable pharmaceutical composition described herein optionally further comprise one or more viscosity modifying agents, emulsifiers, surfactants, humectants, oils, waxes, polymer, preservatives, pH modifying agents (for example a suitable acid or base, for example an organic acid or organic amine base), buffers, antioxidants (for example butylated hydroxyanisol or butylated hydroxytoluene), crystallisation inhibitors (for example a cellulose derivative such as hydroxypropylmethyl cellulose or polyvinylpyrrolidone), colorants, fragrances and taste-masking agents. Such excipients s are well-known, for example as listed in the Handbook of Pharmaceutical Excipients, 7^{th} Edition, Rowe et al.

In certain embodiments the inhalable pharmaceutical composition further comprises a taste-masking agent. The taste masking agent acts to disguise or modulate the unpleasant taste associated with one or more excipients of the composition and/or the niclosamide or pharmaceutically acceptable salt thereof. Taste masking agents are well known. Suitable taste masking agents include, for example a sugar (e.g. sucrose, dextrose, or lactose), a amino acid or amino acid derivative (e.g. arginine, lysine, or monosodium glutamate), an oil (e.g. a natural oil, or plant extract), a sweetener (e.g. aspartame, acesulfame-K, sucralose or saccharin), an organic acid (e.g. citric acid or aspartic acid), or maltodextrin. In certain embodiments the taste-masking agent is present in an amount of up to 10%, up to 5%, or up to 2% by weight of the composition. For example 0.1% to 5% or 0.5% to 2% be weight of the composition.

### Inhalers

The inhalable pharmaceutical composition is administered to the subject by inhalation. The composition is suitably delivered to the subject in an inhalable form using a suitable inhaler. Inhalers are well-known and include dry powder inhalers (DPI), metered dose inhalers (MDI), pressurised metered dose inhalers (pMDI) and nebulizers.

### Nebulizers

Nebulizers are suitable for forming an aerosol of the inhalable pharmaceutical composition. Nebulizers are particularly suitable for forming an aerosol of solution or suspension comprising niclosamide or a pharmaceutically acceptable salt thereof, for example the liquid solutions and suspensions comprising niclosamide or a pharmaceutically acceptable salt thereof described herein. Suitable nebulizers generate a respirable aerosol of the inhalable pharmaceutical composition.

The nebulizer may comprise a reservoir containing the inhalable pharmaceutical composition (e.g. solution or suspension), wherein actuation of the nebulizer delivers a single dose of the composition which is inhaled as a aerosol by the subject. Alternatively, the nebulizer may be a multiple-dose nebulizer wherein a unit dose of the inhalable pharmaceutical composition is loaded into the nebulizer (e.g. via a vial, syringe, capsule, blister-pack or other suitable container) and is administered to the subject as a unit dose of aerosol of the composition.

In certain embodiments the nebulizer is selected from a jet nebulizer, a vibrating mesh nebulizer, an ultrasonic nebulizer. A jet nebulizer utilizes air pressure breakage of a solution or suspension into aerosol droplets. Ultrasonic nebulizers generate an aerosol using shearing of a solution or suspension by a piezoelectric crystal. Vibrating mesh nebulizers comprise a solution or suspension in fluid contact with a vibrating diaphragm mesh. The vibrations of the mesh are used to generate an aerosol of the solution or suspension.

Nebulizers are commercially available and include Respirgard II®, Aeroneb®, Aeroneb® Pro, and Aeroneb® Go produced by Aerogen; AERx® and AERx Essence™ produced by Aradigm; Porta-Neb®, Freeway Freedom™, Sidestream, Ventstream and I-neb produced by Respironics, Inc.; and PARI LC-Plus®, PARI LC-Star®, and e-Flow™ produced by PARI, GmbH.

Preferably the nebulizer is a vibrating mesh nebulizer, for example an e-Flow™ nebulizer. Nebulizers are further disclosed in WO2001032246, WO 01/34232, WO2001056639, WO2001085241, WO2002013896, WO2002064265, WO2003035153, WO2003035152, WO2004004813, WO2004014569, WO2004020029, WO2004028606, WO2004039442, WO2004041336, WO2004041335, WO2004052436, WO2004098689, WO2005032630, WO2005037246, WO2005042075, WO2006108556, WO2006084543, WO2006084546, WO2006128567, WO2007020073, WO2007118557, WO2010097119, WO2016015889, WO2008113651, WO2009135871, WO2010066714, WO2010094767, WO2010097119, WO2010097119, WO2010139730, WO2011134940, WO2012069531, WO2013013852, WO2012168181, WO2014040947, WO2014082818, WO2015091356, WO2015128375, WO2015193432, WO2016026802, WO2016102308, WO2017021441, WO2018167278, WO2019115771 and WO2019202085; incorporated herein by reference thereto.

### Meter Dose Inhalers (MDI)

A propellant driven or pressurised metered dose inhaler (pMDI) releases a metered dose of an aerosol of a solution or suspension comprising niclosamide upon actuation of the inhaler. Suitably the solution or suspension comprising niclosamide, or a pharmaceutically acceptable salt thereof is formulated as a suspension or solution comprising the niclosamide and a suitable propellant such as a halogenated hydrocarbon.

The propellants for use with the MDIs may be any propellants known in the art. Examples of propellants include chlorofluorocarbons (CFCs) such as dichlorodifluoromethane, trichlorofluorometbane, or dichlorotetrafluoroethane; hydrofluoroalkanes (HFAs); nitrogen and carbon dioxide. Suitably the propellant is an HFA, for example hydrofluoroalkane 134a (HFA 134 a), HFA-152a, or hydrofluoroalkane 227ea (HFA 227ea).

The MDI may be actuated with a trigger to release the aerosol for inhalation. Alternatively the MDI may be breath actuated, wherein inhalation by the user triggers release of the aerosol as the user draws in breath.

### Dry Powder Inhalers

Dry powder inhalers (DPI) are suitable for the inhalation of powders comprising niclosamide or a pharmaceutically acceptable salt thereof. The DPI may be a reservoir device wherein the drug is contained within a reservoir in the device and the device delivers a unit dose of the drug from the drug reservoir. Alternatively the DPI may be a metered device wherein a unit dosages of the drug is loaded into the device and inhaled as an aerosol of the powder. Examples of DPI's include those described in A. H. de Boer et al., Expert Opinion on Drug Delivery, 2017, 14:4, 499-512.

DPIs are commercially available and include Novolizer®, Easyhaler®, Pulvinal®, Taifun®, Twisthaler®, Turbuhaler®, Clickhaler®, SkyeHaler®, Airmax®, Spiromax®, Diskhaler®, Diskus®, Spiros®, Taper DPI, Jethaler®, MAGhaler®, Breezhaler® and NEXThaler® inhalers.

### Intranasal Delivery Devices

The intranasal delivery device may be adapted to deliver a solution or suspension to the nasal mucosa. The intranasal delivery device may be a dropper, a metered dose spray pump (e.g. a multi-dose, or a bi-directional multidose spray pump), a squeeze bottle, a single-dose or duo-dose spray device, a nasal pressurized metered-dose inhaler (pMDI), a pulsation membrane nebulizer, a nasal sonic/pulsating jet nebulizer, a vibrating mesh nebulizer, a nasal atomizer or a gas- or electrically-driven atomizer.

Squeeze bottles are generally used to deliver over-the-counter medicines, such as decongestants. By manually squeezing a deformable (e.g plastic) air-filled bottle, the solution is atomized when delivered through a jet outlet.

Metered-dose spray pumps are commonly used for nasal drug delivery. Traditional spray pumps use preservatives to prevent contamination when the emitted liquid is replaced with air. However, more recent devices avoid the need for preservatives by using a collapsible bag, a moveable piston or a compressed gas to replace the emitted liquid, or alternatively use a filter to decontaminate the air. Commercially available nasal spray pumps are sold by Aptar Group.

Single-dose or duo-dose spray devices are intended for one-off or sporadic use, and/or where accurate dosing is important, for example for the administration of expensive drugs and vaccines. Commercially available devices include the MAD Nasal™ Intranasal Mucosal Atomization Device, and the Accuspray™ sold by Becton Dickinson Technologies.

Nasal pressurized metered-dose inhalers (pMDIs) have been developed which use hydrofluoroalkanes (HFAs) as a propellant. Such devices have been approved for the treatment of allergic rhinitis.

Pulsation membrane nebulizers generate an aerosol via a perforated vibrating membrane. Commercially available devices include the VibrENT device sold by PARI Pharma GmbH. Other types of commercially available nebulizers and atomizers include the Atomisor NL11S® sonic (a nasal sonic/pulsating jet nebulizer, DTF-Medical, France) the Aeroneb Solo® (a mesh nebulizer, Aerogen), OptiNose® devices comprising Bi-Directional™ technology, the ViaNase™ electronic atomizer (Kurve Technology Inc.) and nitrogen-driven atomizers (e.g. as sold by Impel Inc.).

In some embodiments, the intranasal delivery device is adapted to deliver a powder to the nasal mucosa. The intranasal delivery device may be a nasal powder inhaler (e.g. which is adapted for nasal delivery), a nasal powder sprayer or a nasal powder insufflator. Commercially available devices include Rhinocort Turbuhaler®, Twin-lizer™, Fit-lizer™ (SNBL), Unidose™ Xtra (Bespak), Monopowder (Aptar group), and the powder Exhalation Delivery System (EDS) sold by OptiNose®.

### Viral Infection

Suitably the inhalable composition of the invention is used to treat a viral infection. The viral infection can be any viral infection that responds to treatment with niclosamide.

For example, the viral infection can be caused by or associated with a virus selected from the families Coronaviridae (e.g. Alphacoronavirus, Betacoronavirus, Gammacoronavirus and Deltacoronavirus), Picornaviridae (e.g. Enteroviruses, such as rhinoviruses, suitable Human rhinoviruses (HRVs)), Flaviviridae (e.g. Zika virus (ZIKV), dengue (e.g. DENV 1-4), West Nile virus (WNV), yellow fever virus (YFV, e.g. yellow fever 17D virus), Japanese encephalitis virus (JEV), Hepatitis C virus (HCV), Filoviridae (e.g. Ebolavirus)), Togaviridae (e.g. Alphaviruses such as Chikungunya virus (CHIKV), Sindbis virus and Ross River virus), Herpes (e.g. γ-herpesvirus, Human herpesvirus 8, herpesvirus 1 and herpesvirus 2) and Adenoviridae (e.g. Human adenoviruses (HAdVs)).

Viruses which infect or which carry out at least one phase of their life cycle or are pathogenic in the respiratory tract are of most interest in the present invention. Such viruses can in some cases enter a subject via the respiratory tract (e.g. they are capable of transmission through inhalation, e.g. via airborne or droplet transmission), and/or they may carry out initial or further stages of replication in the respiratory tract (e.g. upper or lower respiratory tract). Some well-known examples of viruses that are transmitted through airborne or droplet transmission include coronaviruses, influenza virus, parainfluenza virus, adenoviruses, respiratory syncytial virus, human metapneumovirus. Other viruses not consider classical airborne or droplet transmitted virus can in some circumstances be transmitted through the air, e.g. is bodily fluids containing the virus are aerosolised. Furthermore, other viruses that are not transmitted through the air may replicate or be pathogenic in the respiratory tract, and thus can be treated using the inhalable composition of the invention.

Viruses that are transmitted through airborne or droplet transmission and/or which cause viral respiratory disease are of particular interest in the present invention

The inhalable pharmaceutical compositions of the invention are administered by inhalation to provide the treatment or prevention of viral infection. In embodiments the viral infection is caused by or associated with a respiratory virus. Thus it may be that the viral infection is a respiratory tract infection. The viral infection may be an upper respiratory tract infection. The viral infection may be a lower respiratory tract infection, for example a viral infection affecting the lungs.

In some embodiments, the viral infection is caused by or associated with a virus selected from respiratory syncytial virus, influenza virus, parainfluenza virus, human metapneumovirus, coronavirus (e.g. severe acute respiratory syndrome coronavirus (SARS-CoV), severe acute respiratory syndrome coronavirus (SARS-CoV-2), Middle East respiratory syndrome coronavirus (MERS-CoV)), Ebola virus (EBOV), flavivirus, a human rhinovirus (HRVs), human adenovirus (HAdV), and Epstein-Barr virus (EBV).

In some embodiments, the viral infection is a respiratory tract infection (RTI). A respiratory tract infection (RTI) is an infectious diseases involving the respiratory tract. An infection of this type is normally further classified as an upper respiratory tract infection (URI or URTI) or a lower respiratory tract infection (LRI or LRTI). The RTI can be an upper or lower RTI. Lower respiratory infections, such as pneumonia, tend to be far more serious conditions than upper respiratory infections, such as the common cold. The upper respiratory tract is generally considered to be the airway above the glottis or vocal cords, sometimes it is taken as the tract above the cricoid cartilage. This part of the tract includes the nose, sinuses, pharynx, and larynx. Symptoms of URIs can include cough, sore throat, runny nose, nasal congestion, headache, low grade fever, facial pressure and sneezing. The lower respiratory tract consists of the trachea (wind pipe), bronchial tubes, the bronchioles, and the lungs. Lower respiratory tract infections are generally more serious than upper respiratory infections. LRIs are the leading cause of death among all infectious diseases. The two most common LRIs are bronchitis and pneumonia.

The virus can be a RNA virus or a DNA virus. In certain embodiments the viral infection is caused by or associated with an RNA virus. In certain embodiments the viral infection is caused by or associated with a DNA virus. In certain embodiments the viral infection is caused by or associated with a positive-sense strand RNA virus.

In certain embodiments the viral infection is caused by or associated with a virus selected from respiratory syncytial virus, influenza virus, parainfluenza virus, a pneumovirus (e.g. human metapneumovirus), a coronavirus (e.g. severe acute respiratory syndrome coronavirus (SARS-CoV), severe acute respiratory syndrome coronavirus (SARS-CoV-2), Middle East respiratory syndrome coronavirus (MERS-CoV)), human rhinovirus (HRVs), human adenovirus (HAdV).

In some embodiments the virus is an RNA virus that causes or is associated with a RTI.

In some embodiments the viral infection can cause or may be associated with acute respiratory syndrome, e.g. severe acute respiratory syndrome (SARS). Viruses which are known to cause severe acute respiratory syndrome (SARS) include coronaviruses such as a SARS viruses or MERS viruses, e.g. SARS-CoV, SARS-CoV-2 or MERS-CoV. In one embodiment the viral infection causes SARS.

The viruses of the Pneumoviridae family are negative sense, single-stranded, RNA viruses. Two genera within the Pneumoviridae family are Metapneumo virus and Orthopneumovirus. Particular species of Metapneumovirus are avian metapneumovirus (AMPV) and human metapneumovirus (HMPV). Particular species of Orthopneumovirus are Bovine respiratory syncytial virus (BRSV), Human respiratory syncytial virus (HRSV) and Murine pneumonia virus (MPV). Viruses in the Pneumoviridae family are typically transmitted through respiratory secretions and are often associated with respiratory infections. In certain embodiments the viral infection is caused by or associated with Human respiratory syncytial virus (HRSV). Thus it may be that the virus is caused by or associated with a virus selected from: HRSV-A2, HRSV-B1 and HRSV-S2.

Coronaviridae viruses are a family of enveloped, positive-stranded, single-stranded, spherical RNA viruses. The Coronaviridae family includes two sub-families, Coronavirus and Torovirus. The Coronavirus genus has a helical nucleocapsid, and Torovirus genus has a tubular nucleocapsid. Within the Coronavirus sub-family are the following genera: Alphacoronavirus, Betacoronavirus, Gammacoronavirus and Deltacoronavirus. Genera within the Torovirus sub-family are Bafinivirus and Torovirus. In certain embodiments the viral infection is caused by or associated with a coronavirus. Thus is may be that the viral infection is caused by or associated with a virus selected from Alphacoronavirus, Betacoronavirus, Gammacoronavirus and Deltacoronavirus. In a preferred embodiment the viral infection is caused by or associated with a Betacoronavirus.

Human coronaviruses usually cause mild to moderate upper-respiratory tract illnesses, like the common cold, that last for a short amount of time (although some coronaviruses can be deadly). Symptoms may include runny nose, cough, sore throat, and fever. These viruses can sometimes cause lower-respiratory tract illnesses, such as pneumonia. This is more common in people with cardiopulmonary disease or compromised immune systems, or the elderly.

In some embodiments, the viral infection is a common cold. The common cold may be caused by or associated with a virus selected from respiratory syncytial virus (RSV), parainfluenza virus, a pneumovirus (e.g. human metapneumovirus), a coronavirus, rhinovirus (e.g. human rhinovirus, HRVs), adenovirus (e.g. human adenovirus, HAdV), and enterovirus.

Middle East respiratory syndrome coronavirus (MERS-CoV) is a member of the Betacoronavirus genus, and causes Middle East Respiratory Syndrome (MERS). MERS is an acute respiratory illness. About half of the individuals confirmed to have been infected with MERS died. There is no current treatment or vaccine for MERS.

Another member of the Betacornavirus genus is SARS coronavirus (SARS-CoV). SARS-Co-V is the virus that causes severe acute respiratory syndrome (SARS). SARS was first reported in Asia in February 2003. SARS is an airborne virus, and can spread by the inhalation of small droplets of water that an infected individuals releases into the air (for example, by coughing and/or sneezing), touching a contaminated surface and/or by being in close proximity of an infected individual.

In certain embodiments the viral infection is caused by or associated with severe acute respiratory syndrome coronavirus (SARS-CoV), severe acute respiratory syndrome coronavirus 2 (SARS-CoV-2), Middle East respiratory syndrome coronavirus (MERS-CoV), HCoV-229E, HCoV-NL63, HCoV-OC43 and HKU1.

In certain embodiments the viral infection is caused by or associated with a coronavirus that causes severe acute respiratory syndrome (SARS), such as a SARS virus or MERS virus, e.g. SARS-CoV, SARS-CoV-2, or MERS-CoV. Preferably the viral infection is caused by or associated with SARS-CoV-2.

Pathogenic respiratory viral infections can cause disease and symptoms associated with the viral infection. In certain embodiments the inhalable pharmaceutical composition is for use in the prevention or treatment of a disease or condition associated with a respiratory viral infection. Thus is may be that the inhaled composition is for use in the treatment or prevention of a respiratory syndrome caused by or associated with a respiratory viral infection. For example the treatment or prevention of severe acute respiratory syndrome (SARS). Thus it may be that the inhaled composition of the invention is for use in the prevention or treatment of severe acute respiratory syndrome caused by SARS-CoV, SARS-CoV-2, or MERS-CoV, preferably the treatment or prevention of severe acute respiratory syndrome caused by SARS-CoV-2. In certain embodiments the inhaled composition of the invention is for use in the treatment of a respiratory syndrome selected from: pneumonia, influenza and croup. Thus it may be that the inhaled composition is for use in the treatment or prevention of pneumonia caused by a respiratory viral infection.

In a preferred embodiment the inhaled pharmaceutical composition is for use in the treatment of COVID-19.

COVID-19 can be diagnosed by any method known to the skilled person. Samples (e.g., sputum, mucus, sera, nasal aspirate, throat swab, broncho-alveolar lavage or other types of body fluids) from subjects can be obtained and tested for the presence of SARS-CoV-2. Exemplary methods for diagnosing an infection with SARS-Cov-2 include, but are not limited to, detection of a nucleotide sequence of a SARS-CoV-2 virus (e.g. using PCR), detection of a SARS-Cov-2-associated coronavirus antigen, and antibodies or fragments thereof that immunospecifically bind to a SARS-CoV-2-associated coronavirus antigen.

An example of a nucleotide sequence of a SARS-CoV-2 virus is described by Wu et al. (Nature 579, 265-269 (2020) (Genbank accession no. MN908947.3, isolate Wuhan-Hu-1). The subject may be infected with a SARS-CoV-2 virus having a genome sequence which is at least 90%, at least 93%, at least 95%, at least 96%, at least 97%, at least 98%, at least 99%, at least 99.5%, at least 99.6%, at least 99.7%, at least 99.8%, at least 99.9%, at least 99.91%, at least 99.92%, at least 99.93%, at least 99.94%, at least 99.93%, at least 99.95%, at least 99.96%, at least 99.97%, at least 99.98%, or at least 99.99% identical to MN908947.3. The treatment or prophylaxis of any variant of SARS-CoV-2 is encompassed by the invention. No single consistent nomenclature currently exists for SARS-CoV-2 strains, although a number of nomenclatures have been proposed. In some embodiments, the SARS-CoV-2 variant belongs to one of clades S, O, L, V, G, GH, GR or GV (as defined by GISAID “Global phylogeny, updated by Nextstrain”). In some embodiments, the SARS-CoV-2 variant belongs to one of clades 19A, 19B, 20A, 20B, 20C, 20D, 20E, 20F, 20G, 20H or 201 (clades.nextstrain.org, archived in Wayback machine on 19 Jan. 2021). In some embodiments, the SARS-CoV-2 variant belongs to one of the lineages A, B, B.1, E1.1, B1.177 or B.1.1.7 (as proposed by Rambaut et al., Nature Microbiology volume 5, pages 1403-1407(2020)). In some embodiments, the SARS-CoV-2 variant is selected from the group consisting of: the 501.V2 variant (also known as 501.V2, 20H/501Y.V2 (formerly 20C/501Y.V2), VOC-202012/02 (PHE); lineage B.1.351 or “The South African variant”); Cluster 5 (also referred to as AFVI-spike by the Danish State Serum Institute (SSI), believed to have spread from minks); Lineage B.1.1.207; Lineage B.1.1.7 or “Variant of Concern 202012/01” or “the UK variant” (see Chand et al., “Investigation of novel SARS-COV-2 variant, Variant of Concern 202012/01, Public Health England); Lineage B.1.429/CAL.20C; Lineage B.1.525 (also called VUI-202102/03 by Public Health England (PHE) and formerly known as UK1188); and Lineage P.1 (also called Variant of Concern 202101/02 by Public Health England and 20J/501Y.V3 by Nextstrain); Lineage B.1.1.317; Lineage B.1.1.318 and Lineage P.3. In some embodiments, the SARS-CoV-2 variant is one which carries one or more of the following mutations: D614G; E484K; N501Y; S477G/N; P681H.

Subjects with viral infections can develop serious conditions associated with the viral infection. Treatment of a subject with a respiratory viral infection using the inhaled composition of the invention may prevent or treat a condition selected from: sepsis, pneumonia or organ failure associated with a respiratory viral infection. In some embodiments the inhaled composition is for use in the treatment or prevention of sepsis caused by or associated with the respiratory viral infection. In some embodiments the inhaled composition is for use in the treatment or prevention of pneumonia caused by or associated with the respiratory viral infection. The pneumonia may be viral pneumonia or bacterial pneumonia (e.g. bacterial pneumonia caused by or associated with secondary bacterial infection in the lung of a subject). Thus it may be that the inhaled composition of the invention is for use in the treatment or prevention of viral pneumonia.

In certain embodiments the viral infection is caused by or associated with influenza virus. The influenza virus may be type A; type B, type C or type D. Type A and B viruses cause seasonal epidemics in humans, while type A viruses have caused several pandemics. Type C viruses generally cause mild illness and are not generally associated with epidemics. Type D viruses primarily affect cattle. Type A viruses can be divided into subtypes based on their surface proteins hemagglutinin (H) and neuraminidase (N). There are 18 different hemagglutinin proteins (designated H1 to H18) and 11 different neuraminidase proteins (designated N1 to N11). This gives 198 potential influenza A type combinations, although only 131 subtypes have been detected to date. The viral infection may be caused by or associated with a Type A influenza virus selected from H1N1, H1N2, H2N2, H3N2, H5N1, H7N7, H9N2, H7N2, H7N3, H10N7, H7N9 and H6N1. Type B viruses are not classified into subtypes, but can be categorised into lineages. Type B viruses may belong to either the B/Yamagata or B/Victoria lineage.

In certain embodiments the inhaled composition of the invention is for use in the treatment or prevention of bacterial pneumonia caused by or associated with a respiratory viral infection (i.e. the treatment of bacterial pneumonia secondary to the viral infection). Thus it may be that the inhaled composition of the invention is for use in the treatment or prevention of Streptococcus pneumoniae. In a particular embodiment the inhaled composition of the invention is for use in the treatment or prevention of Staphylococcal pneumonia.

The antibacterial effects of niclosamide may provide a particularly effective treatment secondary infections such as bacterial pneumonia. The inhaled compositions of the invention have both antiviral and antibacterial action, and accordingly can be used to treat both viral and bacterial pathogens in the lung. Accordingly, also provided is an inhaled composition of the invention for use as an antibacterial agent to target a bacterial infection that is secondary to a respiratory viral infection (e.g. Gram-positive bacteria). Thus it may be that the inhaled composition of the invention is for use in the treatment of secondary bacterial infection in a subject with a respiratory viral infection, wherein the secondary bacterial infection is caused by or associated with a Gram-positive bacteria, preferably a bacteria selected from one or more of: S. aureus (e.g. MRSA), S. pneumoniae, H. influenzae and M. catarrhalis.

In certain embodiments provides inhaled composition of the invention for use as an antibacterial agent to target one or more bacteria which can cause or contribute to pneumonia. In this embodiment it may be that the bacteria targeted are Gram-positive bacteria, for example one or more of S. aureus (e.g. MRSA), S. pneumoniae, H. influenzae and M. catarrhalis. Thus it may be that the inhaled composition eradicates or reduces the bacteria can cause or contribute to pneumonia.

In certain embodiments the inhaled pharmaceutical composition of the invention is for use in the treatment or prevention of a symptom of a viral infection (e.g. SARS-CoV-2) selected from fever (e.g. a fever above 38° C.), cough, sore throat, shortness of breath, respiratory distress, and pneumonia. Suitably the inhaled composition is used to treat severe acute respiratory syndrome (SARS).

In certain embodiments the inhaled pharmaceutical composition of the invention may for use in reducing mucus production and/or secretion caused by or associated with a respiratory viral infection.

In certain embodiments the inhaled pharmaceutical composition of the invention may for use in reducing bronchoconstriction caused by or associated with a respiratory viral infection.

Subjects with viral infections, particularly respiratory viral infections, are prone to developing pulmonary fungal infections. Niclosamide is known to have antifungal properties (Garcia et al., Sci Rep. 2018; 8(1):11559. Published 2018 Aug. 1. doi:10.1038/s41598-018-29973-8). Accordingly, the inhaled composition of the invention may provide an effective treatment of opportunistic pulmonary fungal infections associated with a viral infection. In certain embodiments there is provided an inhaled pharmaceutical composition of the invention for use in the treatment of a pulmonary fungal infection caused by or associated with a viral infection (e.g. a respiratory viral infection). The fungal infection may be an opportunistic pulmonary fungal infection. In certain embodiments the pulmonary fungal infection is a Candida Spp. infection, for example a Candida albicans. In certain embodiments the inhaled composition of the invention is for use in the treatment or prevention of pulmonary candidiasis. Particularly the inhaled composition of the invention is for use in the treatment or prevention of pulmonary candidiasis in a subject with a viral infection, preferably a respiratory viral infection.

Niclosamide has anti inflammatory properties, accordingly the inhaled pharmaceutical composition of the invention may be beneficial in reducing, ameliorating or treating pulmonary inflammation associated with respiratory viral infections, because niclosamide has both antiviral and anti inflammatory properties.

In certain embodiments there is provided an inhaled pharmaceutical composition of the invention for use in the treatment or prevention of pulmonary inflammation caused by or associated with respiratory viral infection. For example the inhaled composition may reduce or eliminate inflammation of tissues in the respiratory tract.

In certain embodiments the inhaled pharmaceutical composition is for use in preventing or repressing pro-inflammatory cytokines caused by or associated with the viral infection. Thus it may be that the inhaled pharmaceutical composition reduces one or more of CRP leukocytes, IL1B, IL-6, IL-10, IL-2, IFNγ, IP10, MCP1, GCSF, IP10, MCP1, MIP1A, and/or TNFα, particularly reducing serum CRP. In some embodiments the inhaled pharmaceutical composition reduces levels of IL-6 in a subject with a respiratory viral infection.

Viral infections (including, but not limited to SARS CoV-2) can induce cytokine release syndrome (CRS) (also known as a cytokine storm syndrome (CSS)). CRS is a systemic inflammatory response triggered by the viral infection and results in the sudden release of large numbers of pro-inflammatory cytokines which can damage organs and in particular may lead to respiratory failure. Recent publications suggest that cytokine storm is observed in some patients with severe forms of COVID-19 (Zhang et al, International Journal of Antimicrobial Agents https://doi.org/10.1016/j.ijantimicag.2020.105954, available online 29 Mar. 2020). In some embodiments there is provided an inhaled composition of the invention for use in the prevention, repression or treatment of cytokine release syndrome in a subject with a respiratory viral infection (e.g. a subject infected with SARS-CoV2, SARS or MERS).

In certain embodiments the inhaled pharmaceutical composition has an antiviral effect on the virus. For example by preventing or inhibiting viral replication. Without wishing to be bound by theory, it is believed that the inhaled composition can act as an antiviral by to inhibiting or preventing viral replication in at least the respiratory tract of a patient. Accordingly, in some embodiments the composition of the invention is for use in preventing or inhibiting viral replication in a subject with a viral infection (e.g. a respiratory viral infection) In some embodiments the compositions may reduce or eliminate the viral load in the subject.

It will be appreciated that the combined treatment of multiple conditions using the inhalable composition of the present invention provides significant advantages over the use of multiple therapies.

In some embodiments of the invention the aerosol or solution is used as an anti-viral and as an anti-inflammatory and/or as an anti-bacterial. Thus, in some embodiments the aerosol or solution is used as at least a dual therapy or triple therapy. Thus, in some embodiments the aerosol or solution can be used to target viral infection and inflammation and/or bacterial infection for the treatment of an RTI, for example in a coronaviral infection such as SARS. In some embodiments the aerosol or solution is used as an anti-viral, as an anti-inflammatory and as an anti-bacterial for the treatment of an RTI, for example in a coronaviral infection such as SARS.

In some embodiments of the invention the inhalable composition is used to treat a viral infection as an antiviral (e.g. to prevent viral replication) and to further provide one or more of the following additional therapeutic effects:

anti-bacterial;

anti-inflammatory;

reduction or prevention of bronchoconstriction/to cause bronchodilation; and/or

reduction of mucus production and/or secretion.

The subject infected with a respiratory viral infection may be asymptomatic at the early stages of a viral infection. Treatment of asymptomatic subjects may prevent the viral infection becoming symptomatic and/or developing diseases or medical conditions associated with the respiratory viral infection. Accordingly also provided is an inhaled pharmaceutical composition of the invention for use in the treatment of an asymptomatic subject infected with a virus. In some embodiments the virus is a respiratory virus (e.g. a SARS virus such as SARS-CoV-2).

The halogenated salicylanilides such as niclosamide may provide a particularly effective treatment against viral infections such as SARS-CoV-2. Evidence has suggested that niclosamide possesses broad spectrum antiviral properties, including against SARS-CoV-2 (Xu et al., J ACS Infect Dis 2020; Wu et al., Antimicrob Agents Chemother 2004:48:2693-6). It has been suggested that the mode of action of niclosamide may include inhibition of autophagy, viral replication and receptor-mediated endocytosis of SARS-CoV2 (Pindiprolu et al., Medical Hypotheses 140 (2020) 109765).

Some viral infections become contagious before symptoms emerge in a subject infected with the virus, for example as is the case with SARS-Cov-2. This can result in high rates of transmission of the virus in a population, because the infected host does not know that they are contagious and inadvertently spreads the virus through social contact etc. Transmission of a virus by asymptomatic subjects can be particularly dangerous after an initial infection is contained in a population, because asymptomatic, but contagious, subjects can trigger a resurgence of infections and a “second wave” of viral infection. Using the inhaled composition of the invention to treat an asymptomatic subject with a viral infection may reduce the time that a subject is contagious by, for example reducing or eliminating the virus from the subject and/or to speed up seroconversion in the subject (i.e. the production of antibodies to the virus by the subject's immune system). Treatment using the inhaled composition of the invention may reduce the viral shedding from the subject, thereby making the subject less contagious. Viral shedding refers to the number of virus leaving the body of the subject in for example mucous droplets resulting from coughing or sneezing, or present in other excreta.

Accordingly, in some embodiments there is provided an inhaled composition of the invention for use in the treatment of a viral infection in an asymptomatic subject, wherein the treatment reduces or eliminates the viral load in the subject. In some embodiments there is provided an inhaled composition of the invention for use in the treatment of a viral infection in an asymptomatic subject, wherein the treatment accelerates seroconversion in the subject. In some embodiments there is provided an inhaled composition of the invention for use in the treatment of a viral infection in an asymptomatic subject, wherein the treatment reduces inter-subject transmission of the virus. In some embodiments there is provided an inhaled composition of the invention for use in the treatment of a viral infection in an asymptomatic subject, wherein the treatment reduces viral shedding. The viral infection may be SARS-CoV-2.

In some embodiments, there is provided an inhaled composition of the invention for use in the treatment of SARS-CoV2 in an asymptomatic or mildly symptomatic subject. The subject may have tested positive for SARS-CoV-2 (e.g. via a PCR test). The treatment may be started within 0-5 days, or within 1-3 days, of the positive test result (day 0 being the day the test result is received by the subject). In some embodiments the subject is not taking, or has not recently taken (e.g. within the previous 30 or 60 days), immunosuppressive drugs. The subject may not be at a higher risk from SARS-CoV-2. Administration of the inhaled composition to an asymptomatic or a mildly symptomatic subject may prevent or reduce the risk of the subject developing symptoms of mild, moderate or severe COVID-19, particularly symptoms of moderate to severe COVID-19. Treatment of an asymptomatic or a mildly symptomatic subject may also reduce the number of members of the subject's household who become infected with SARS-CoV-2. In some embodiments, administration of the inhaled composition to an asymptomatic or a mildly symptomatic subject reduces the time-weighted change (reduction) from baseline through day 10. In other words, the inhaled composition may reduce the risk of, or prevent, the progression of the disease. In an asymptomatic subject, “baseline” refers to the subject having no symptoms.

The detection of a viral infection in an asymptomatic subject may be achieved using known testing methods, for example tests which detect the presence of the virus in saliva samples such as real-time reverse transcription polymerase chain reaction (rRT-PCR) or PCR methods. In some embodiments there is provided a formulation of the invention for use in the treatment or prophylaxis of a subject who has received a positive diagnosis of a viral infection, such as COVID-19 (SARS-CoV-2). The subject may be suffering from mild, moderate or severe COVID-19, or they may be asymptomatic. Prophylactic treatment of subjects who have not received a positive test for the presence of SARS-CoV-2 infection, or who have not been tested, is also envisaged.

Symptoms of COVID-19 are non-specific and the disease presentation can range from no symptoms (asymptomatic) to severe pneumonia and death. The clinical progression of COVID-19 shows a biphasic pattern. The first phase is characterized by fever, cough, fatigue and other systemic symptoms like dizziness and headache, shortness of breath, rhinorrhoea, sore throat, diarrhoea and inappetence. Fever is seen in most of the patients with an estimated median duration of 10 days (95 confidential intervals after onset of symptoms (Chen et al. Clinical progression of patients with COVID-19 in Shanghai, China. J Infect. 2020; 80(5):e1-e6.).

As the disease progresses into the second phase, symptoms begin to relieve in most of the patients and radiological improvement occurs in parallel. In line with body temperature reduction, patients also become PCR negative with their upper respiratory tract samples (mean time to viral clearance is around 11 days). There is however a small sub-group of patients (˜5%) which present with respiratory failure, septic shock, and multiorgan dysfunction, resulting in higher fatality rates. Persistent fever, lung damage and diseases progression can be partially explained by uncontrolled viral replication. The persistence of COVID-19 can also induce excessive but aberrant non-effective response which is associated with cytokine storm.

Patients with “mild” COVID-19, as used herein, are subjects with a score of 2, 3 or 4 on the modified WHO scale described below. Subjects may be ambulatory or hospitalized. They show symptoms of COVID-19 that could include fever, cough, sore throat, malaise, headache, shortness of breath, muscle pain, loss of taste and/or smell, ocular symptoms (e.g. one or more of conjunctival hyperemia, chemosis, epiphora, or increased secretions) and/or gastrointestinal symptoms (e.g. diarrhoea) of variable intensity and they can either have no or mild signs of viral pneumonia. They may display a limitation of daily activities. They do not need oxygen treatment.

Patients with “moderate” COVID-19, as used herein, are subjects with a score of 5 on the modified WHO scale described below. Subjects are hospitalized with COVID-19 needing treatment with oxygen by mask or nasal prongs. They show symptoms that could include fever, cough, sore throat, malaise, headache, muscle pain and/or gastrointestinal symptoms of variable intensity. They have a moderate pneumonia.

Patients with “severe” COVID-19, as used herein, are subjects with a score of 6, 7 or 8 on the modified WHO scale described below. These subjects require intensive care and/or mechanical ventilation or extra-corporeal membrane oxygenation. Such patients may display hypoxemia, extrapulmonary hyper-inflammation, severe pneumonia, vasoplegia, respiratory failure, cardiopulmonary collapse and/or systemic organ involvement. Markers of systemic inflammation (e.g. IL-2, IL-6, IL-7, granulocyte colony-stimulating factor, macrophage inflammatory protein 1-a, tumor necrosis factor-α, C-reactive protein, ferritin, and/or D-dimer) may be elevated.

In any of the embodiments described herein, the subject may be hospitalized.

By targeting patients at a stage where viral replication is high but has not yet led to severe tissue damage, the treatment may reduce duration of symptoms, minimize contagiousness, and prevent progression of severity and poor outcome.

Accordingly, in some embodiments there is provided an inhalable pharmaceutical composition of the invention for use in the treatment of a viral infection in a subject suffering from mild or moderate COVID-19. In some embodiments, the subject suffering from mild or moderate COVID-19 is hospitalized. In some embodiments, the subject is suffering from moderate COVID-19 and is hospitalized. In some embodiments, the subject is suffering from mild COVID-19 and the composition is administered intranasally. In some embodiments, the subject is suffering from moderate COVID-19 and the composition is administered intranasally. In some embodiments, the subject is suffering from moderate COVID-19 and the composition is administered intraorally by inhalation. In some embodiments, the subject is suffering from moderate COVID-19 and the composition is administered intranasally and intraorally by inhalation. In some embodiments, the subject is suffering from mild or moderate COVID-19 and is hospitalized, wherein the inhalable composition is administered intranasally and intraorally by inhalation. It may be that administration of the composition is for preventing, or reducing the likelihood of, progression of the disease, e.g. from mild to moderate or from moderate to severe COVID-19. In some embodiments, the subject is identified as being at risk of disease progression. For example, the subject may be identified as being at risk of progressing from mild to moderate, or from moderate to severe COVID-19. In some embodiments, the subject may be identified as being at risk of an increase in the subject's score on the modified WHO scale, as described below. A skilled doctor or nurse will be capable of identifying at-risk subjects. For example, a subject who is at risk of disease progression may be identified based on one or more factors, which may include clinical parameters (such as the subject's respiratory status, blood oxygen saturation, temperature, severity of flu-like symptoms, chest X-ray or other scans, inflammatory biomarker levels, viral load and the presence of underlying conditions) and, optionally, non-clinical parameters (such as the subject's age and gender).

The treatment may reduce or eliminate the viral load in the subject (e.g. the viral load in sputum or blood), for example, it may be that the treatment reduces the viral load in the nasal cavity. It may be that the treatment reduces the viral load in the lungs of a subject. In some embodiments, the treatment reduces the time taken to cure the disease, relative to a patient not treated with the formulation of the invention. The treatment may avoid the need for hospitalization in patients with mild COVID-19, or reduce hospitalization time for patients with moderate COVID-19. The treatment may prevent the progression of the disease. For example, the treatment may prevent progression from mild to moderate, or from moderate to severe COVID-19. The treatment may prevent an increase in a subject's score on a modified WHO scale as described below. The treatment may reduce or eliminate the need for oxygen therapy. The treatment may increase blood oxygen levels. The treatment may prevent or reduce the risk of respiratory failure. The treatment may reduce the time for viral clearance from a subject. The treatment may reduce or eliminate viral colonization. For example the treatment may reduce or eliminate viral colonization in the nasal cavity. It may be that treatment reduces or eliminates viral colonization in the lungs.

In some embodiments, there is provided an inhalable pharmaceutical composition of the invention for use in the treatment of a viral infection in subject suffering from severe COVID-19.

The treatment may reduce the time the patient spends in intensive care, relative to a patient not treated with the formulation of the invention. In some embodiments, the treatment improves the efficacy of a co-administered drug, such as an anti-inflammatory agent. The treatment may reduce the severity of symptoms, the recovery time, and/or the long term effects of the disease.

In some embodiments, there is provided an inhalable pharmaceutical composition of the invention for use in the treatment of a viral infection (e.g. COVID-19), wherein said treatment includes one or more of the following: a reduction in the severity of flu-like signs and symptoms (e.g. temperature); an improvement in the respiratory status of the subject as assessed by oximetry (blood oxygen saturation); an improvement in the NEWS2 score; an improvement in the score on the modified WHO ordinal scale, the FDA COVID-19 questionnaire (Table 13) and/or the WHO 11-point ordinal scale (Table 14), as described herein; reduction or elimination of pulmonary inflammation and/or edema; an improvement in respiratory function; a reduction in shortness of breath; a reduction in the time to viral clearance; a reduction in the time to discharge from hospital; reduced viral load; a reduction in inflammatory serum markers (e.g. CRP, procalcitonin). In some embodiments, treatment results in subjects having an improvement in the score on the modified WHO ordinal scale by 1 to 6 grades, 2 to 5 grades, or 3 to 4 grades. In some embodiments, treatment results in subjects having an improvement in the NEWS2 score by from 1 to 6 points, from 2 to 5 points, or from 3 to 4 points.

Also provided is a prophylactic treatment wherein the inhaled composition of the invention is administered to a subject to prevent or reduce the risk of contracting a viral infection. In certain embodiments there is provided an inhaled composition of the for use in reducing the risk of, or preventing, a subject contracting a viral infection. Such prophylactic treatments may be particularly beneficial to subjects that may be exposed to high levels of a virus, for example doctors, nurses, social workers and other healthcare workers that are caring for people with viral infections, or may be more likely to come into contact with people with viral infections; and workers who are exposed to the general population e.g. in large numbers, such as teachers, nursery staff, transport workers and shop assistants.

In some embodiments, the formulation of the invention is prophylactically administered. In some embodiments, the formulation is prophylactically administered to a subject who has been, or is suspected as having been, in close proximity with a person who is diagnosed as being infected with SARS-CoV-2. For example, family, co-workers and/or other close contacts of an infected individual, who are identified as having being at risk of exposure to the virus, may be administered the formulation of the invention as a prophylactic treatment. The close contacts of the infected individual may be identified via a tracking and tracing program, such as a government-operated program. Prophylactic treatment of subjects after suspected exposure to an infected person may be beneficial in preventing further spread of the virus. In some embodiments, the subject starts the prophylactic treatment no more than 7 days, no more than 6 days, no more than 5 days, no more than 4 days, no more than 3 days, no more than 2 days or no more than 24 hours after the exposure, or suspected exposure, to the infected individual. The close contacts may be subjects who are identified as having been in close proximity to the infected individual and include, for example, subjects who share a home, office, school or mode of transport with the infected individual, those who have taken part in a sport or other social activity with the infected individual, and those who may have come into close proximity with the infected individual in a public space such as a restaurant, bar, café, transport terminal, library, hospital or other medical facility, or shop. Preferably, prophylactic treatments may be administered intranasally. It may be that prophylactic treatment is administered to the general public, for example in the case of an epidemic.

The treatments and prophylactic treatments described herein may also be particularly beneficial to subjects who are at a higher risk from COVID-19. These subjects include: those with an existing disease or condition, such as diabetes (such as Type I or Type II diabetes mellitus, in particular poorly controlled diabetes), cancer, heart disease (such as heart failure, coronary artery disease and cardiomyopathy), hypertension (in particular poorly controlled hypertension), cerebrovascular disease, vasculitis, SCID, sickle cell disease (including sickle cell anaemia), thalassemia, pulmonary fibrosis, interstitial lung disease, chronic lung disease such as COPD, asthma (particularly moderate to severe asthma) and cystic fibrosis, emphysema, bronchitis, kidney disease (including chronic kidney disease, diabetic nephropathy, membranous nephropathy and glomerular disease, such as glomerulonephritis, minimal change nephropathy, focal segmental glomerulosclerosis, IgA nephropathy, primary membranous nephropathy, membranoproliferative glomerulonephritis and lupus nephritis), chronic liver disease, hepatitis, a genetic immune disease, autoimmune disease (including systemic lupus erythematosus (SLE), Anti-GBM, rheumatoid arthritis, psoriatic arthritis, connective tissue disease, spondyloarthritis, polymyalgia rheumatica, inflammatory bowel disease (including Crohn's disease and ulcerative colitis), coeliac disease, aplastic anaemia, Addison's disease, Graves' disease, Hashimoto's thyroiditis, myasthenia gravis, autoimmune vasculitis, pernicious anaemia and Sjögren's syndrome), hepatitis, a condition affecting the brain or nerves (such as Parkinson's disease, motor neurone disease, multiple sclerosis, dementia, mental illness or cerebral palsy, and subjects who have suffered from a stroke) a muscle wasting condition, or a severe or profound learning disability. Subjects at high or moderate risk from COVID-19 also include subjects who have a weakened immune system, for example due to a disease, condition or treatment. These subjects include: subjects who have had a tissue transplant, such as an organ transplant (including kidney, liver, lung and/or heart transplant recipients); subjects who have had an organ (e.g. their spleen) removed; subjects receiving (or who have received) chemotherapy, immunotherapy, antibody therapy or radiotherapy; subjects receiving protein kinase inhibitors or PARP inhibitors; subjects receiving (or who have received) cancer treatment; subjects who have had a blood, bone marrow or stem cell transplant (e.g. in the last 6-12 months); subjects who are immunocompromised, including subjects taking immunosuppressants (e.g. ciclosporin, tacrolimus, azathioprine, mycophenolate mofetil or mycophenolic acid, belatacept, methotrexate, tocilizumab, abatacept, leflunomide, prednisolone, anti-TNF (e.g. infliximab, adalimumab, etanercept), cyclophosphamide, rituximab or alemtuzumab), or steroids, subjects with HIV or AIDS); subjects on dialysis (including haemodialysis and peritoneal dialysis); subjects who are very obese (with a BMI of at least 30, at least 40 or above); and subjects who are pregnant. Also included are subjects who smoke; care home residents; staff working in care homes for adults over 50, 60, 65, 70, 75 or 80 years of age; frontline health and/or social care workers; black and minority ethnic (BAME) groups; and subjects who are over 50, 60, or 70 years of age, in particular subjects over 75, 80, 85 or 90 years of age.

Thus, in some embodiments there is provided an inhalable pharmaceutical composition of the invention for use in reducing the risk of, or preventing, a subject contracting a viral infection (e.g. COVID-19), wherein the subject is at a higher risk from COVID-19, for example wherein the subject is selected from the groups defined above.

In some embodiments, there is provided the formulation for use in prophylaxis of non-infected subjects who are at a higher risk from COVID-19, such as a subject selected from the groups defined above. It may be that the prophylaxis is for reducing the risk of the subject contracting symptomatic or non-symptomatic COVID-19 infection. The prophylaxis may be for reducing the risk of mortality, and/or the severity of symptoms (should the subject contract COVID-19). It may be that the prophylaxis is for reducing the risk of the subject contracting moderate or severe COVID-19.

In some embodiments, the prophylaxis reduces the risk of the subject contracting a secondary infection (e.g. a secondary bacterial infection), wherein the subject is at a higher risk from COVID-19, for example wherein the subject is selected from the groups defined above. It may be that the prophylaxis reduces the risk of mortality, or the severity of, the secondary infection.

The composition of the invention is therefore particularly suited to prophylactic treatment of higher risk groups, i.e. subjects who are at a higher risk from infection, such as COVID-19. “Subjects who are at a higher risk from COVID-19”, also referred to as “higher risk subjects” or “higher risk patients”, include subjects who have a weakened immune system (i.e. they are immunocompromised), which reduces the body's ability to fight infections and other diseases. It also reduces the subject's ability to recover from infections. Higher risk subjects may have a higher risk of contracting COVID-19, and/or a higher risk of suffering from more severe and/or a longer duration of infection. Higher risk subjects may also be more vulnerable to different types of infections e.g. secondary infections.

In some people, COVID-19 can cause symptoms which last for weeks or months after the infection has gone. This is known as “long COVID”, or “post-COVID-19 syndrome”. Subjects with long COVID may experience symptoms for at least 4, 6, 8, 10, 12, 16, 20 or 24 weeks, or at least 3, 4, 6, 8, 10, 12 months after the infection has gone. It may be that the subject experiences symptoms for at least 8 or at least 12 weeks. Symptoms of long COVID may include one or more of: extreme tiredness (fatigue); shortness of breath; chest pain or tightness; problems with memory and/or concentration (‘brain fog’); difficulty sleeping (insomnia); dizziness; tingling sensations in hands and/or feet (‘pins and needles’); joint pain; depression; anxiety; tinnitus; earaches; nausea; diarrhoea; stomach aches; loss of appetite; elevated temperature; palpitations; chest pains; joint and/or muscle pain; cough; headaches; sore throat; changes to taste and/or smell; skin rashes; or hair loss.

In some embodiments, there is provided a formulation of the invention for use in treating, preventing or reducing the incidence of long COVID. It may be that treatment with a formulation of the invention reduces the duration of long COVID, and/or reduces the number and/or severity of symptoms of long COVID.

In certain embodiments there is provided a composition of the invention for use in treating a viral infection in a subject (e.g. COVID-19), wherein the subject is selected from the groups defined above. In some embodiments the composition is administered intranasally.

In some embodiments, said treatment comprises administering the inhalable pharmaceutical composition of the invention in combination with a further therapeutic or prophylactic agent. The further therapeutic or prophylactic agent may be an anti-viral agent (e.g. Remdesivir), an anti-inflammatory agent (e.g. a steroid, such as dexamethasone), an immunosuppressive agent, a neutralizing antibody or an anti-thrombotic agent. Combination therapy may be particularly beneficial for subjects with a severe viral infection (e.g. severe COVID-19).

### Bacterial Infections

In some embodiments, the compositions and methods described herein are used in the treatment of bacterial infections, for example pulmonary bacterial infections. The bacterial infection may be a primary infection (i.e. the primary or only disease the subject is suffering from), or the bacterial infection may be secondary infection associated with another (primary) infection (e.g. a viral infection) or an inflammatory disease.

In some embodiments, the compositions and methods described herein are used for the treatment or prevention of a bacterial infection in the lungs of a subject having a chronic lung condition, such as cystic fibrosis (CF), non-cystic fibrosis bronchiectasis (non-CFBE), chronic obstructive pulmonary disorder (COPD), or non-tuberculous mycobacterial (NTM) pulmonary infection.

In some embodiments, the bacterial infection is caused by a gram-positive bacteria, such as: Corynebacterium diphtheriae, Corynebacterium ulcerans, Streptococcus pneumoniae, Streptococcus agalactiae, Streptococcus pyogenes, Streptococcus milleri; Streptococcus (Group G); Streptococcus (Group C/F); Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus. In some embodiments, the bacteria is a gram-positive anaerobic bacteria, by non-limiting example these include Clostridium difficile, Clostridium perfringens, Clostridium tetini, and Clostridium botulinum. In some embodiments, the bacterial infection is caused by an acid-fast bacteria, by non-limiting example these include Mycobacterium tuberculosis, Mycobacterium avium, Mycobacterium intracellulare, and Mycobacterium leprae. In some embodiments, the bacterial infection is caused by an atypical bacteria, by non-limiting example these include Chlamydia pneumoniae and Mycoplasma pneumoniae.

In some embodiments, the bacterial infection is caused by a bacterium selected from: S. aureus, S. pneumoniae, H. influenzae, M. catarrhalis and S. pyogenes.

### Bacterial Skin Infections

Also provided is a composition of the invention for the treatment (preferably the topical treatment) of a skin infection caused by or associated with Gram-positive bacteria.

In some embodiments the composition of the invention is for use in the treatment of from impetigo, sycosis barbae, superficial folliculitis, paronychia erythrasma, acne, secondary infected dermatoses, carbuncles, furonculosis, ecthyma, cellulitis, erysipelas, necrotising fasciitis and secondary bacterial skin infections of wounds, dermatitis, scabies, diabetic ulcer, rosacea or psoriasis. For example the composition of the invention may be for use in the topical treatment of an atopic dermatitis lesion, wherein said lesion is infected with Gram-positive bacteria.

In some embodiments the composition of the invention is for use in the topical prevention or treatment of an outer ear infection caused by or associated with a Gram-positive bacteria.

It may be that the Gram-positive bacteria is a Staphylococcus spp., Streptococcus spp. or Propionibacterium spp. The Gram-positive bacteria may be a Staphylococcus spp. or Streptococcus spp. The Gram-positive bacteria may be selected from Staphylococcus aureus or Streptococcus pyogenes. The Gram-positive bacteria may be Propionibacterium spp., for example Propionibacterium acnes. It may be that the Gram-positive bacteria is not a propionibacteria e.g. that it is not Propionibacterium acnes.

In some embodiments, the population of Gram-positive bacteria includes coccus Gram-positive bacteria. In some embodiments, the Gram-positive bacteria are from the Streptococcus or Staphylococcus genus.

In some embodiments, the Gram-positive bacteria are from the Streptococcus genus. It may be that the Gram-positive bacteria are Streptococcus selected from Streptococcus pneumoniae, Streptococcus pyogenes, Streptococcus suis, Streptococcus agalactiae or Streptococcus viridans.

In some embodiments, the Gram-positive bacteria are Streptococcus pyogenes.

In some embodiments, the Gram-positive bacteria are from the Staphylococcus genus. It may be that the Gram-positive bacteria are Staphylococcus selected from Staphylococcus epidermidis, Staphylococcus aureus, Staphylococcus saprophyticus or Staphylococcus lugdunensis. In some embodiments, the coccus Gram-positive bacteria are Staphylococcus aureus (e.g. methicillin-resistant Staphylococcus aureus).

It may be that the population of Gram-positive bacteria includes antibiotic-resistant Gram-positive bacteria. It may be that the Gram-positive bacteria is an antibiotic resistant strain. For example, the Gram-positive bacteria described herein may be resistant to an antibiotic other than a halogenated salicylanilide (for example the bacteria is resistance to a drug other than closantel, rafoxanide, oxyclozanide or niclosamide, or a pharmaceutically acceptable salt or solvate thereof).

It may be that the Gram-positive bacteria is resistant to a drug selected from fusidic acid, mupirocin, retapamulin, erythromycin, clindamycin and a tetracycline (for example tetracycline, minocycline or doxycycline).

It may be that the Gram-positive bacteria is resistant to a drug selected from erythromycin, clindamycin or a tetracycline (for example tetracycline, minocycline or doxycycline).

It may be that the Gram-positive bacteria is resistant to a drug selected from fusidic acid, mupirocin and retapamulin.

It may be that the bacteria is resistant to a drug selected from fusidic acid, mupirocin, retapamulin, erythromycin and clindamycin.

The composition of the invention may be for use to decolonise a subject carrying a Gram-positive bacteria (including any of the Gram-positive bacteria described herein, for example MRSA). Such decolonisation may be effective in preventing or reducing the spread of infection to other subjects particularly in a hospital environment. Decolonisation may also prevent or reduce the risk of surgical site infections resulting from surgical or medical procedures carried out on the patient or at the site of medical devices such as catheters or IV lines or cannula. Accordingly the formulation of the invention may be for use in the decolonisation of a subject prior to carrying out a surgical procedure on the subject, wherein the formulation is applied topically to the subject. Such surgical procedures include, for example elective surgical procedures such as hip or knee replacement. In one embodiment the composition of the invention may be for use in the decolonisation of a subject prior to dialysis. Pre-dialysis decolonisation may prevent or reduce the risk of infection associated with dialysis such as vascular line infection or catheter related bloodstream infections (CRBSI) infections. Decolonisation may be achieved by topically administering the gel composition comprising the halogenated salicylanilide to sites on the subject which are colonised by the Gram-positive bacteria. It is known that a common site for bacterial colonisation such as MRSA is the nose. Accordingly, the formulation of the invention may be applied topically to the nose. Particularly the formulation of the invention may be applied to the anterior nares (the inner surface of the nostrils).

### Fungal Infections

In a further aspect, a composition of the invention is for use in the treatment of a pulmonary fungal infection. Suitably in this embodiment the formulation of the invention is administered by inhalation.

In certain embodiments a composition of the invention is for use in the treatment of a pulmonary fungal skin infection. Suitably the formulation of the invention is topically applied.

Fungal lung and/or skin infections may be caused by Candida sp., Aspergillus sp., and/or Pneumocystis jirovecii. In some embodiments, the formulations and methods described herein are for treating a fungal infection caused by Candida albicans, Candida tropicalis, Candida krusei, Candida glabrata, Aspergillus fumigatus, Aspergillus flavus, Aspergillus niger, and/or Pneumocystis jirovecii.

### Inflammatory Disease

In another aspect, a composition of the invention is for use in the treatment of an inflammatory disease. In certain embodiments the inflammatory disease is a pulmonary inflammatory disease. Pulmonary inflammatory diseases include, but are not limited to, pulmonary inflammatory disease is selected from the group consisting of: asthma, chronic obstructive pulmonary disease (COPD), pulmonary fibrosis, pneumonia, interstitial lung disease, sarcoidosis, bronchiolitis obliterans, pneumonitis, acute respiratory distress syndrome (ARDS), bronchiectasis, cystic fibrosis, idiopathic pulmonary fibrosis, radiation induced fibrosis, silicosis, asbestos induced pulmonary or pleural fibrosis, acute lung injury, usual interstitial pneumonia (UIP), Chronic lymphocytic leukemia (CLL)-associated fibrosis, Hamman-Rich syndrome, Caplan syndrome, coal worker's pneumoconiosis, cryptogenic fibrosing alveolitis, obliterative bronchiolitis, chronic bronchitis, emphysema, Wegner's granulamatosis, lung scleroderma, silicosis, asbestos induced pulmonary and/or pleural fibrosis.

The term “pulmonary fibrosis”, includes all interstitial lung disease associated with fibrosis. In some embodiments, pulmonary fibrosis includes the term “idiopathic pulmonary fibrosis” or “IPF”. In some embodiments, pulmonary fibrosis, by non-limiting example, may result from inhalation of inorganic and organic dusts, gases, fumes and vapours, use of medications, exposure to radiation or radiation therapy, and development of disorders such as hypersensitivity pneumonitis, coal worker's pneumoconiosis, chemotherapy, transplant rejection, silicosis, byssinosis and genetic factors. Exemplary pulmonary inflammatory diseases for the treatment or prevention using the formulations and methods described herein include, but are not limited, idiopathic pulmonary fibrosis, pulmonary fibrosis secondary to systemic inflammatory disease such as rheumatoid arthritis, scleroderma, lupus, cryptogenic fibrosing alveolitis, radiation induced fibrosis, chronic obstructive pulmonary disease (COPD), sarcoidosis, scleroderma, chronic asthma, silicosis, asbestos induced pulmonary or pleural fibrosis, acute lung injury and acute respiratory distress (including bacterial pneumonia induced, trauma induced, viral pneumonia induced, ventilator induced, non-pulmonary sepsis induced, and aspiration induced). In some embodiments the formulations and methods of the invention may be for use in the treatment or prevention of secondary bacterial or viral infections associated with a pulmonary inflammatory disease (e.g. a secondary bacterial infection associated with COPD).

In some embodiments, the compositions and methods described herein are used to treat or slow down the progression of or prevent asthma. Asthma may be associated with or caused by environmental and genetic factors. Asthma is a common chronic inflammatory disease of the airways characterized by variable and recurring symptoms, reversible airflow obstruction, and bronchospasm. Symptoms include wheezing, coughing, chest tightness, and shortness of breath. Non-limiting examples of asthma include, but are not limited to, allergic asthma, non-allergic asthma, acute severe asthma, chronic asthma, clinical asthma, nocturnal asthma, allergen-induced asthma, aspirin-sensitive asthma, exercise-induced asthma, child-onset asthma, adult-onset asthma, cough-variant asthma, occupational asthma, steroid-resistant asthma, or seasonal asthma.

In some embodiments, the compositions and methods described herein can treat or slow down the progression of or prevent lung inflammation. Lung inflammation may be associated with or contribute to the symptoms of bronchitis, asthma, lung fibrosis, chronic obstructive pulmonary disorder (COPD), and pneumonitis. The halogenated salicylanilide niclosamide has been shown to reduce mucus production and secretion, as well as bronchoconstriction, in a mouse model of asthma. In addition, niclosamide was found to be a potent inhibitor of the Cl⁻ channels TMEM16A and TMEM16F, which contribute to the release of mucus and inflammatory mediators. Niclosamide may therefore be suitable for the treatment of inflammatory airway diseases such as cystic fibrosis, asthma and COPD (Cabrita et al., JCI Insight 2019; 4(15):e128414).

In some embodiments, the compositions and methods described herein are used to treat or prevent clinical signs and symptoms of, or infections associated with, cystic fibrosis. Cystic fibrosis (CF) is a genetic disorder that affects mostly the lungs, and involves frequent bacterial infections. Approximately 85% of CF patients have chronic, recurrent P. aeruginosa infection, which significantly contributes to lung function decline and mortality. Long-term issues include difficulty breathing and coughing up mucus as a result of these frequent lung infections. Thus, in some embodiments, the formulations and methods are used to treat a bacterial infection, such as a P. aeruginosa infection, associated with cystic fibrosis. In some embodiments, the compositions and methods are used to treat a bacterial infection associated with cystic fibrosis, wherein the bacterial infection is caused by or associated with a Gram-positive bacteria (e.g. bacteria selected from S. aureus, S. pneumoniae, H. influenzae, M. catarrhalis and S. pyogenes).

In preferred embodiments the pulmonary inflammatory disease is treated by inhaling a composition of the invention (e.g. by inhalation of an aerosol of a composition of the invention).

### Dosages and Dosage Regimens

The dosage and dosing regimen of the inhalable pharmaceutical composition of the invention will depend upon a number of factors that may readily be determined by a physician, for example the severity of the viral infection, the responsiveness to initial treatment, the mode of administration and the particular infection being treated. Examples of suitable doses, dosing volumes and dosing frequencies are set out in the brief summary of the disclosure above.

When the inhaled composition of the invention is administered to the subject using an inhaler (e.g. a nebulizer) not all of the dose loaded into the inhaler will reach the lungs because, for example some drug will be entrained in the device, some of the drug may not enter the mouth or nose of the subject and some mat become entrained in the oral or nasal cavity and not penetrate into the airways (e.g. the lung). Reference to the doses of the inhalable compositions described herein refer to the dose of niclosamide or pharmaceutically acceptable salt thereof which is loaded into the inhaler, or is metered by the inhaler before the inhaler is actuated. The dose inhaled by the subject may be for example 10%, 15%, 20% or 25% lower that the pre-actuation dose.

The total daily dose of niclosamide administered to the subject may comprise one or more unit doses. The total daily dose may be from 5 to 1000 mg, from 6 to 800 mg, from 8 to 700 mg, from 10 to 500 mg, from 15 to 400 mg, from 30 to 300 mg, from 50 to 250 mg, from 100 to 200 mg or from 120 to 250 mg of niclosamide, or a pharmaceutically acceptable salt thereof.

In some embodiments the total daily dose is from 1 to 50 mg, from 1.5 to 40 mg, from 2 to 30 mg, from 2.5 to 20 mg, from 3 to 15 mg, from 3.5 to 12 mg, from 4 to 10 mg, from 4.5 to 9 mg, from 5 to 8.5 mg, from 5.5 to 8 mg, from 6 to 7.5 mg or from 6.5 to 7 mg of the halogenated salicylanilide or salt thereof (e.g. niclosamide or niclosamide ethanolamine). In some embodiments the total daily dose is 5.6 mg niclosamide ethanolamine, corresponding to 4.7 mg niclosamide free base.

The dose may be delivered to the subject via multiple modes of administration. In some embodiments, a first dose may be administered intranasally (e.g. using a nasal spray device) and a second dose may be administered intraorally (e.g. using a nebulizer). It will be appreciated that the first dose may be administered after the second dose, or vice versa. For example, in embodiments wherein the pharmaceutical composition of the invention is in the form of a solution, a volume of from 50 to 250 μl, or from 100 to 200 μl (e.g. 130-150 μl) per nostril may be administered intranasally, and a volume of from 1 to 10 ml, from 2 to 8 ml or from 3 to 7 ml (e.g. 4-6 ml) may be administered intraorally (e.g. via a nebulizer). In some embodiments, a volume of 140 μl per nostril is administered intranasally, and a volume of 3 ml is administered intraorally (e.g. via a nebulizer). Both solutions may be dosed twice daily.

The pharmaceutical composition of the invention may be administered once per day, or multiple times (e.g. 2, 3 or 4 times) per day. In some embodiments the composition is administered twice daily.

The total daily volume administered to the subject may be from 200 μl to 20 ml, from 300 μl to 19 ml, from 500 μl to 18 ml, from 1 ml to 17 ml, from 2 ml to 16 ml, from 3 to 15 ml, from 4 to 14 ml, from 5 ml to 12 ml or from 8 ml to 10 ml of a solution of the invention. In some embodiments, the pharmaceutical composition of the invention is a solution containing from 0.1 to 5%, from 0.5 to 5%, from 1 to 4%, from 1.5 to 3% (e.g. from about 1 to 2%) of niclosamide, or a pharmaceutically acceptable salt thereof.

The pharmaceutical composition of the invention may be administered to the subject over a number of consecutive days or weeks. For example, the composition may be administered one or more times daily over a period of from 3 days to 6 weeks, from 7 days to 4 weeks from 10 days to 3 weeks or from 14 to 18 days. In some embodiments, the formulation is administered over a period of from 1 week to 1 year, from 2 weeks to 9 months, from 4 weeks to 6 months, from 6 weeks to 4 months, or from 2 to 3 months. For example, the treatment may be administered for up to 6 to 9 months. In some embodiments, the formulation is administered to the subject twice daily for up to 10, 14 or 28 days. It will be appreciated that the dosing period will be determined by the type and severity of the disease being treated, or whether the formulation is being administered prophylactically. For example, for the treatment of chronic conditions (e.g. COPD, asthma, and infections associated with cystic fibrosis), or moderate or severe cases of COVID-19, the treatment duration may be longer (e.g. at least 4 weeks, at least 6 weeks, at least 8 weeks or at least 12 weeks). It may be that treatment is continued until the subject has recovered.

In some embodiments, the subject is intranasally administered 100-200 μl (e.g. 120-180 μl or 130-160 μl) per nostril of a 1% solution of niclosamide ethanolamine, twice per day. In a preferred embodiment, the subject is intranasally administered 140 μl per nostril of a 1% solution of niclosamide ethanolamine, twice per day. Additionally, or alternatively, the subject may be administered from 1 to 10 ml, from 2 to 8 ml, from 3 to 6 ml or from 4 to 5 ml of a nebulised solution of 1% niclosamide ethanolamine, twice per day.

It will be appreciated that the dose of the formulation and/or the dosage regime may be selected by the skilled person depending on a number of factors such as, but not limited to, the severity of the disease, the age of the subject and/or the presence of any underlying conditions.

In some embodiments, the formulation is administered to a subject for the treatment or prophylaxis of COVID-19. In some embodiments wherein the subject is suffering from mild COVID-19, the subject is asymptomatic, or the subject is being treated prophylactically (e.g. a subject in a high-risk group, or a close contact of an infected individual), the formulation may be administered one or more times daily for a period of no more than 21 days, no more than 18 days, no more than 16 days, no more than 14 days, no more than 12 days or no more than 10 days. In some embodiments wherein the subject is suffering from moderate or severe COVID-19, the formulation may be administered one or more times daily for a period of at least 7 days, at least 10 days, at least 14 days, at least 21 days or at least 28 days.

As will be appreciated the doses and dosage regimens set out in this section may be used with any of the formulations of the invention. In a preferred embodiment the formulation of the invention used in any of the doses and dosage regimens described herein and in this “dosage and dosage regimens” is a liquid formulation comprising: about 0.5 to about 5% niclosamide ethanolamine (w/w); about 95 to about 99.5% PEG 400 (w/w).

### Combination Therapies

The formulation of the invention may be used alone to provide a therapeutic effect. The formulation of the invention may also be used in combination with one or more additional therapeutic agents.

In some embodiments the additional therapeutic agent is selected from one or more of:
an antiviral agent (e.g. remdesivir, a HIV protease inhibitor (e.g. lopinavir or ritonavir), or a 3CL protease inhibitor (e.g. PF-07304814);
a vaccine (e.g. a COVID-19 vaccine), examples of vaccines include weakened or inactivated viral vaccines, replicating or non-replicating viral vector vaccines, nucleic acid vaccines (RNA or DNA vaccines), protein subunit vaccines or virus-like particle vaccines;
bronchodilators, e.g. short acting beta agonists (e.g. albuterol, epinephrine or levalbuterol), or long acting beta agonists (e.g. formoterol, salmeterol or vilanterol);
anticholinergics (e.g. ipratropium);
leukotriene modifiers (e.g. montelukast, zafirlukast, or zileuton);
long-acting bronchodilators (e.g. tiotropium);
anti-inflammatory agents (e.g. steroids, which may be intravenous, oral or inhaled steroids (e.g. dexamethasone, budesonide); non-steroidal anti-inflammatory agents (e.g. ibuprofen, naproxen, ketoprofen or carprofen, a COX-2 inhibitor such as celecoxib), an anti-inflammatory antibody (e.g. benralizumab, dupilumab, mepolizumab, omalizumab, reslizumab);
an antibacterial agent, for example a Gram-positive or Gram negative antibiotic;
an anti-viral antibody (e.g antibodies that act against the spike proteins of a corona virus such as SARS-CoV-2 (e.g. LY-CoV555, LY-CoV016, AZD7442, REGN10933, or REGN10987); and antibodies from subjects that have previously been infected with a virus (e.g. convalescent plasma therapies); or a combination of any two or more thereof.

Such combination treatment may be achieved by way of the simultaneous, sequential or separate dosing of the individual components of the treatment. Such combination products employ the formulation of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the formulation of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the halogenated salicylanilide (e.g. niclosamide or pharmaceutically acceptable salt thereof) present in the formulation of the invention and an approved or otherwise published dosage range(s) of the other pharmaceutically active agent(s).

### Examples

### Example 1: Non-Aqueous Niclosamide Formulations

The inhalable compositions shown in Table 1 were prepared:

TABLE 1

| | Composition | | |
|---|---|---|---|
| Raw material | Formulation | Formulation | Formulation |
| INCI or PhEur | A | B | C |
| name (trade name) | % (w/w) | % (w/w) | % (w/w) |
| Niclosamide ethanolamine | 0.50 | 1.50 | 5.00 |
| Saccharin | 0.04 | 0.12 | 0.40 |
| PEG 400 | 99.46 | 98.38 | 94.60 |

The formulations were prepared as follows. Niclosamide ethanolamine (50 mg for Formulation A, 150 mg for Formulation B and 500 mg for Formulation C), PEG 400 (9.946 g for Formulation A, 9.838 g for Formulation B and 9.46 g for Formulation C) and saccharin (4 mg for Formulation A, 12 mg for Formulation B and 40 mg for Formulation C) were weighed in blue cap bottles. The mixture was agitated or stirred at room temperature until a clear solution formed. Typically, a solution is obtained following stirring for a few hours (e.g. 1 to 12 hours).

The final formulations were protected from light prior to further use.

The following inhalable compositions shown in Table 2 could also be prepared in a similar method:

TABLE 2

| | Niclosamide | | |
|---|---|---|---|
| | ethanolamine | PEG 400 | Saccharin |
| Composition | (% (w/w)) | (% (w/w)) | (% (w/w)) |
| 1 | 0.1 | 99.892 | 0.008 |
| 2 | 0.25 | 99.973 | 0.02 |
| 3 | 0.75 | 99.19 | 0.06 |
| 4 | 1.00 | 98.92 | 0.08 |
| 5 | 2.00 | 97.84 | 0.16 |
| 6 | 3.00 | 96.76 | 0.24 |
| 7 | 4.00 | 95.68 | 0.32 |
| 8 | 10.00 | 89.20 | 0.80 |
| 9 | 0.10 | 99.90 | 0.00 |
| 10 | 0.25 | 99.75 | 0.00 |
| 11 | 0.50 | 99.50 | 0.00 |
| 12 | 0.75 | 99.25 | 0.00 |
| 13 | 1.00 | 99.00 | 0.00 |
| 14 | 1.50 | 98.50 | 0.00 |
| 15 | 2.00 | 98.00 | 0.00 |
| 16 | 3.00 | 97.00 | 0.00 |
| 17 | 4.00 | 96.00 | 0.00 |
| 18 | 5.00 | 95.00 | 0.00 |
| 19 | 10.00 | 90.00 | 0.00 |

### Example 2: Nebulisation of Non-Aqueous Niclosamide Formulations

Certain compositions disclosed herein may be nebulised by an electronic nebuliser (for example, an eFlow® electronic nebuliser (PARI)), to provide an aerosol which could be administered to a patient via inhalation.

The drug delivery efficiency of the nebulised formulations of Example 1 may be assessed by breath simulation, and the droplet size and distribution pattern may be determined by laser diffraction (as described in US 2009/0304604 A1).

### Example 3: Clinical Trials

The following clinical trial may be performed using a niclosamide composition described herein, such as the 5% NEN formulations defined in Example 1.

### Study Design

Phase 1 (Dose-Finding): An ascending dose scaling study in adult healthy volunteers (HV) to test the safety of three formulations with increasing doses of NEN formulations according to Example 1.

Phase 2 (Assessment in Patents): A clinical study to assess safety and explore efficacy of four times daily (QID) treatment with the selected dose of NEN (in a formulation according to Example 1) in adult patients with moderate COVID-19. The final dosing frequency can be adjusted by the Safety Monitoring Committee (SMC) based on Phase 1 data.

27 healthy volunteers and 44 subjects with COVID-19 may be enrolled in five sequential cohorts:

Phase 1—Dose-Finding (3 Cohorts):
Cohort 1: 9 healthy volunteers, 7 to receive a single dose of a 0.5% NEN formulation according to Example 1 (4 mL) and 2 to receive placebo.
Cohort 2: 9 healthy volunteers, 7 to receive a single dose of a 1.5% NEN formulation according to Example 1 (4 mL and 2 to receive placebo.
Cohort 3: 9 healthy volunteers, 7 to receive a single dose of a 5.0% NEN formulation according to Example 1 (4 mL) and 2 to receive placebo.

Phase 2—Final Dose Assessment:
Cohort 4: 4 COVID-19 patients to be treated with a formulation according to Example 1 in the selected concentration of two times a day (BID) (2 subjects) or QID (2 subjects) for 15 days. The final dosing frequency can be adjusted by the SMC based on Phase 1 data.
Cohort 5: 40 COVID-19 patients, 20 to be treated with a formulation according to Example 1 in the selected concentration, and 20 to receive placebo, QID for 15 days. The final dosing frequency can be adjusted by the SMC based on Phase 1 data.

Screening and enrolment for Cohorts 1 to 3 will be initiated in parallel, while dosing will be done sequentially. Dosing will begin with Cohort 1. Once data from minimum 8 subjects is available, the safety will be assessed by a Safety Monitoring Committee (SMC) before initiating dosing of subjects in Cohort 2. Similarly, the SMC will review the data from minimum 8 subjects in Cohort 2 before opening for dosing in Cohort 3. Based on the data from the first three cohorts the SMC will select the most appropriate dose. For all four HV cohorts, one subject will be dosed with a NEN formulation according to Example 1 (open-label) the first day and followed for 24 hours while admitted at the clinic to confirm safety of the new dose, if safety concerns are observed the SMC will be involved to adjudicate, if no safety concerns are observed or the SMC judges it safe to continue dosing, the remaining 8 subjects in each cohort can be randomised and dosed (double-blinded) with an interval of at least one hour. Once safety and PK data are available from all Phase 1 cohorts, the SMC will assess safety and review PK data to confirm safety, recommend a dose, and if relevant confirm or adjust the QID regimen proposed for Phase 2.

Should subjects experience coughing, discomfort and/or pain associated with inhalation of the nebulised investigational product (IP) (to such an extent the investigator assess it will be an issue for administration), the investigator may decide that inhaled lidocaine can be administered prior to inhalation of IP. The first subject in Cohort 1 should be dosed without lidocaine, if issues are observed in Cohort 1 or later cohorts the investigator can decide to administer lidocaine for the remaining subjects in the cohort currently being dosed and the SMC shall subsequently make a decision on whether to implement this for all remaining IP administrations in the study.

Upon selection of dose and confirmation of safety in Phase 1, Phase 2 will commence with opening for enrolment and treatment of patients in Cohort 4. The purpose of Cohort 4 is to confirm safety and tolerability of dosing in patients. For this purpose, all four subjects in Cohort 4 will be treated with a NEN formulation according to Example 1 (open-label, no placebo) and enrolled in one centre to ensure experience is collected and one investigator together with the SMC can assess safety across the cohort. Treatment will start with two patients treated BID, who shall be followed for 48 hours. In case safety concerns are observed with a possible, probable or definite relationship with IP, the SMC will be involved to adjudicate. If no safety concerns are observed, or the SMC judges it safe to continue, the last two subjects in the cohort can be initiated on QID treatment. Once safety data is available for 4 days of treatment of the subjects in Cohort 4, it will be assessed by the SMC to confirm safety in COVID-19 patients before initiating enrolment of subjects in Cohort 5. Subjects in Cohort 5 will be enrolled in multiple centres in a randomized, double-blind, parallel group cohort to ensure an unbiased assessment of safety and efficacy.

For HVs in Phase 1 screening may be performed up to 21 days before initiation of study treatment (a nasopharyngeal swap will be collected between 1 to 3 days before dosing to confirm that HV subjects are not infected with COVID-19). For COVID-19 patients, screening may be performed up to 2 days prior to the initiation of study treatment.

For HVs to qualify for enrolment in Phase 1, they cannot be smokers, should be in good general heath and have a normal medical history excluding any chronic disease of the investigator's judgment as well as minimum 80% of predicted lung function, including Forced Expiratory Volume in 1 second (FEV1), Total Lung Capacity (TLC), Carbon Monoxide Diffusion capacity (DCO), Fractional Exhaled Nitric Oxide (FeNO), and a 6-Minute Walking Test (6-MWT) with pulse oximetry, finally, ECG and chest X-ray must be normal (see Exclusion criteria for full details).

For subjects to be eligible for Phase 2, they must hospitalised with COVID-19 confirmed by a positive SARS-CoV2 test (can be analysed according to standard at the local laboratory). Eligible patients will have moderate disease defined as need for hospitalisation but requiring no more than 5 L oxygen (O₂)/minute, not requiring ventilation, and not being admitted to an intensive care unit (ICU). Finally, eligible subjects cannot currently be treated with other exploratory anti-viral treatments or other investigational products.

In Phase 1, investigational product (IP) or placebo will be a single ascending dose administered by qualified study staff, after which the subject will be followed for 24 hours in the clinic and return for a final check 48 hours after dosing.

In Phase 2, IP or placebo will be administered by a nurse or investigator in the hospital BID or QID for fifteen days.

In Phase 1 (HVs), a general physical examination and serum chemistry (including inflammatory parameters) and haematology sampling will be performed at screening and 48 hours after dosing. If first screening visit is conducted more than 3 days before dosing, the subject must come to the clinic 1 to 3 days before dosing for a nasopharyngeal swap (to confirm no infection with SARS-CoV2) and sampling for serum chemistry and haematology. In terms of respiratory function, safety will be assessed on the basis spirometry (vital capacity and FEV1) as well as pulse oximetry performed pre-dose as well as 1, 3, 6, 12, 24, and 48 hours dosing. TLC, DCO, and FeNO (FeNO is only measured before dosing as part of confirming subjects have normal lung function) will be measured and a 6-MWT with pulse oximetry will be conducted during the screening period (between ICF and dosing) and on Day 2 after dosing (the day of dosing is designated as Day 0). ECGs will be captured at pre-dose, 3, 6, and 24 hours after dosing, while vital signs (systemic blood pressure, resting heart rate (RR), pulse and body temperature) will be measured at pre-dose, 1, 3, 6, 12, 24, and 48 hours post dose. AEs will be collected through-out the study period. Finally, an oropharyngeal swap for detection of viruses and bacteria will be taken pre-dose and 48 hours after dosing for post hoc exploratory analysis of potential changes in the microbiome.

In Phase 2, (patients), a general physical examination and serum chemistry and haematology sampling will be performed at screening, pre-dose, Day 8 and Day 15. In addition, safety will be assessed on the basis of daily oximetry measurements and daily assessment of the clinical respiratory status. Finally, ECGs will be collected pre-dose, 24, and 48 hours after dosing, and on Days 8, 15, and AEs will be collected through-out the study period.

Blood samples for PK analysis will be collected pre-dose, %, 1, 1%, 2, 3, 6, 12, and 24 hours after first dose in Phase 1 (HVs), and pre-dose, 24, and 48 hours, and on Days 8 and 15 in Phase 2 (patients). Finally, an oropharyngeal swap for detection of viruses and bacteria will be taken pre-dose and on Day 15 for post hoc exploratory analysis of potential changes in the microbiome.

In Phase 2, efficacy will be explored based on daily assessment of clinical respiratory status, pulse oximetry (also collected for safety), and body temperature and other flu symptom descriptors. Pre-dose (if feasible), on Day 8 and Day 15 6-MWT with oxygen uptake measurement will be performed. In addition, nasopharyngeal swabs will be taken every other day (and analysed centrally by PT-PCR) and Day 8 and Day 15 (analysed by BioFire® to detect both virus and bacteria), samples for serum inflammatory biomarker analysis (primary markers: CRP, leukocytes; exploratory markers for post hoc analysis: IL1B, IFNγ, IP10, MCP1, GCSF, MIP1A, TNFα (Huang et al., “Clinical features of patients infected with 2019 novel coronavirus in Wuhan, China”, The Lancet, Vol. 395, Issue 102223, p 497-506, 15 Feb. 2020)) collected pre-dose, 48 hours after dosing, and on Days 8 and 15. Finally, if a chest X-ray or CT scan has been collected during hospitalisation, a similar image should be captured on Day 15.

Patients in Phase 2 are considered cured of COVID-19 if the following criteria are met for 72 hours:
Clinical respiratory status: normal (no symptoms, no need for oxygen therapy)
No fever
Normal oxygen saturation
2 successive nasopharyngeal swaps tested negative for SARS-CoV-2

If a patient is cured treatment with IP (or placebo) should be stopped (and the subject may be discharged from the hospital if so decided by the investigator). The subject should still come to the hospital for the Day 15 tests as outlined in the schedule of events (to avoid spreading the virus in case of a relapse the subject must come for a nasopharyngeal swap to be tested for SARS-CoV2 on Day 14 and can only come for the Day 15 visit if confirmed negative).

When reviewing the safety data from cohorts 1 to 4 the SMC will re-evaluate the safety assessments and determine if the data suggests adjusting the sampling schedule to collect the data required for assessment.

### Inclusion Criteria

Subjects are only eligible if they fulfil all criteria for inclusion:

### 1. Signed Informed Consent Form (ICF).

2. Male or nonpregnant and nonlactating female who is abstinent or agrees to use effective contraceptive methods throughout the course of the study. Females must have a negative urine beta-human chorionic gonadotropin hormone (hCG) pregnancy test at Day 1. (Women who are postmenopausal (the menopause is defined as the time when there has been no menstrual periods for 12 consecutive months and no other biological or physiological cause can be identified) or who had tubal ligation/hysterectomy do not need to have a urine or serum pregnancy test and do not need to agree to use contraception). Acceptable birth control methods are the following:
Intrauterine device in place for at least 3 months.
Stable hormonal contraceptive for at least 3 months prior to the Day 1 and continuing through study completion.
   3. Normal ECG (including QTcF<450 ms).

### Additionally, for Cohorts 1, 2 and 3 (HVs):

4. Age 18 and <65 years at the time of signing ICF.
5. Normally active and in good health by medical history and physical examination.
6. Negative alcohol breath test and drugs of abuse test before dosing.
7. Minimum 80% of predicted lung function, including FEV1, TLC, DCO, FeNO, and 6-MWT with pulse oximetry.
8. Normal chest X-ray.

### Additionally, for Cohorts 4 and 5 (Patients):

9. Age ≥18 and <80 years at the time of signing ICF.
10. Hospitalised with COVID-19 confirmed by a positive SARS-CoV2 test.
11. Moderate disease defined as requiring no more than 5 L oxygen (O₂)/minute, not requiring ventilation, and not being admitted to an intensive care unit (ICU).
12. Prior to infection with SARS-CoV-2, normally active and otherwise in good health by medical history and physical examination as determined by investigator's judgment.

### Exclusion Criteria

Subjects who meet any of the following criteria are not eligible to participate in this study:

1. Enrolment in a NEN study in the previous 6 months.
2. Allergy or history of significant adverse reaction to niclosamide or related compounds, to any of the excipients used, or to lidocaine.
3. Underlying condition that may interfere with inhalation of the IP.
4. Current acute or chronic condition (including COPD or other severe respiratory disease, CV disease, diabetes mellitus, obesity, malignant and autoimmune diseases) unless considered clinically irrelevant and stable by the investigator.
5. Severe renal impairment (GFR<29 mL/min. or hepatic impairment (reduced albumin)
6. The presence of a condition which renders the subject “vulnerable” as defined by GCP or of a condition the investigator believes would interfere with the ability to provide consent, or comply with study instructions, or that might confound the interpretation of the study results or put the subject at undue risk.
7. Known difficulty undergoing venipuncture or poor venous access.

### Additionally, for Cohorts 1, 2, and 3 (HVs):

8. Whole blood donation or loss (>400 mL) within 90 days before the dosing of IP.
9. History of any malignancy except subjects with adequately treated basal cell/squamous carcinomas of the skin.
10. Smoke or regular use of any form of nicotine product incl. e-cigarette, snuff, chewing tobacco, nicotine gum, etc. for the previous 6 months.
11. Consumed alcohol in the 24 hours prior to first dose.

### Prior or Concomitant Therapy

12. Any systemic and inhaled therapies 5 half-lives prior to first dose of a NEN formulation according to Example 1 (Hormone replacement therapy for postmenopausal women and hormonal anticonception are allowed).
13. Exposure to any IP within either 5 half-lives or 30 days (whichever is longer) prior to first dose of NEN formulation according to Example 1.

### Additionally, for Cohorts 4 and 5 (COVID-19 Patients):

14. Active or acute viral infection (other than SARS-CoV-2), and/or bacterial infection in the nasal area.
15. Severe COVID-19 defined as requiring more than 5 L oxygen/minute, ventilation and/or admission in an ICU.

### Prior or Concomitant Therapy

16. Current or prior (after COVID-19 diagnosis) exposure to exploratory anti-viral treatments or other IP.

### Administration

Qualified staff will administer 4 mL 0.5%, 1.5%, 5.0% NEN formulation according to Example 1, or placebo once daily in Phase 1 and BID or QID (Cohort 4) and QID (Cohort 5) for 15 days in Phase 2.

Maximum dose to be tested is 5% NEN formulation according to Example 1 QID.

In case coughing, discomfort or pain is experienced by subjects inhaling the IP, inhaled lidocaine may be administered before dosing the IP.

Inhalation is performed by using an prEN 13544-1 certified nebulizer, and sufficient measures will be taken to prevent that dosing infected subjects with a nebulizer results in spread of SARS-CoV2, e.g., by using a nebulizer with spacer or other device to ensure that exhaled air and sputum from the subject cannot be aerosolized or by dosing the inhalation inside an airtight mask.

### Duration of Study

Subject participation in the study (excluding the screening period) is approximately 3 days for Cohort 1 to 3 (HVs) and up to 15 days for Cohorts 4 and 5 (patients) (not including potential follow-up of ongoing SAEs or pregnancies).

### Efficacy Variables

In Phase 2, efficacy will be assessed based on:
Change in clinical respiratory status (including need for oxygen therapy) (daily).
Eradication of SARS-CoV-2 (in the nasopharynx) (every other day).
Change airflow and blood oxygen saturation (daily).
Change in body temperature and other flu symptoms (daily).
Oxygen uptake in 6-MWT (Day 8 and 15).
Chest X-ray or CT scans (Day 15).
Change in serum inflammatory biomarkers (primary marker: CRP, other markers to be collected and explored post hoc: IL1B, IFNγ, IP10, MCP1, GCSF, MIP1A, and TNFα (Huang et al.)) (Day 2, 8 and 15).

### Endpoints and Criteria for Evaluation

Primary Endpoint—Day 15:
Safety assessment of COVID-19 patients treated QID for 15 days.

Secondary Endpoints:
Safety assessment of HVs dosed for one day.
Safety assessment of COVID-19 patients treated BID or QID in daily observation and on Day 2, and Day 8.
Change in clinical respiratory status (on a scale from 0—no signs/symptoms, to 4—very severe, need for intubation) at end of treatment (daily).
Conversion rates between clinical respiratory statuses (daily).
Time to remission of respiratory symptoms (daily).
Time to independence from oxygen therapy (daily).
Change in resting blood oxygen saturation (daily).
Sequential Organ Failure Assessment (SOFA) score (from 0 to 24) (daily).
Share of subjects admitted to ICU (daily).
Time to reduction in fever or other flu symptoms (daily).
SARS-Cov-2 eradication time (measured in the nasopharynx) (every other day).
Change in SARS-Cov-2 viral load (measured in the nasopharynx) (every other day).
Reduction in pulmonary edema/inflammation as assessed by chest X-ray or CT scan (Day 15).
Inflammatory serum biomarker (CRP, leukocytes) normalisation (Day 2, 8, and 15).
Change in oxygen uptake in 6-MWT (Day 8 and 15).

Exploratory End Points (to be Analysed Post Hoc):
Nasopharyngeal microbiome changes (Day 2 for HVs, Day 15 for patients).
Exploratory serum biomarkers of COVID-19 associated inflammation (IL1B, IFNγ, IP10, MCP1, GCSF, MIP1A, and TNFα).

Key PK Parameters:
Maximum quantity of active drug molecules in blood (Cₘₐₓ).
Time to reach maximum level (Tₘₐₓ).
Area Under the Curve of drug level in blood versus time (AUC).

### Efficacy Analysis

The exploratory efficacy endpoints including change in clinical respiratory status, share of subjects developing acute respiratory distress syndrome, time to remission of respiratory symptoms, time to independence from oxygen therapy, SOFA score, reduction in fever or other flu symptoms, reduction in pulmonary edema/inflammation, SARS-Cov-2 eradication time, change in primary inflammatory serum biomarkers (CRP, leukocytes), change in blood oxygen saturation will be presented in tables as well as graphically over time from baseline to Day 10 with last observation carried forward (LOCF). In addition, shift tables will be provided between baseline and each time point for the categorical variables. The cumulative distribution function (CDF) of clinical respiratory status changes from baseline will be plotted to identify where the best separation between treatment and placebo occurs.

The same analyses as above will be repeated in the Per Protocol (PP) analysis set for all above primary and secondary endpoints using observed cases only. The PP analysis set includes data from Cohort subjects who were randomised and had no important protocol deviations affecting efficacy assessment throughout the IP administration period (not including healthy volunteers).

### Example 4: Antibacterial Effects Against Bacteria Causing Pneumonia

### Microorganisms

Bacterial strains were chosen for their relevance regarding lung infections, such as pneumonia: Staphylococcus aureus, methicillin-resistant Staphylococcus aureus (MRSA), Streptococcus pneumoniae, Haemophilus influenzae, Moraxella catarrhalis and Streptococcus pyogenes. S. aureus and S. pyogenes strains are as defined in WO 2016/038035.

Strains were conserved in Luria Bertani (LB) Broth (S. aureus) or Brain Heart Infusion (BHI) (S. pyogenes) supplemented with glycerol 15% (v/v) at −80° C., and reactivated by isolation on LB (S. aureus) or BHI (S. pyogenes) agar plates. Strains were cultivated in Mueller Hinton (MH) Broth-cation adjusted (S. aureus) or BHI (S. pyogenes). All strains were cultivated aerobically (microaerobically for S. pyogenes strains) at 37° C.

The following tests were performed to assess the antibacterial activity of niclosamide in vitro:

### Minimum Inhibitory Concentration (MIC) Assay

The MIC was determined according to the method described in WO 2016/038035.

### Results

TABLE 3
MIC values in μg/mL for niclosamide
using the above described assay.

| Species | MIC (μg/mL) |
|---|---|
| Multi-drug resistant strains of | 0.06-0.5 |
| S. aureus (including MRSA) | |
| (226 isolates) | |
| S. pneumoniae | 0.25 |
| H. influenzae | 0.5 |
| M. catarrhalis | 0.12 |
| S. pyogenes | 0.125 |

The MIC of niclosamide was ≤0.5 μg/mL against all targeted strains.

The results in Table 3 show that niclosamide is effective against a range of bacteria, including bacteria commonly associated with lung infections. Accordingly, the inhalable compositions comprising niclosamide may be effective in the treatment or prevention of secondary bacterial lung infections associated with respiratory viral infections. The data in Table 3, together with the data showing that niclosamide is active against SARS CoV-2 suggests that the inhalable pharmaceutical compositions described herein may be effective in the treatment or prevention of respiratory viral infections such as SARS CoV-2 and diseases associated with respiratory viral infections such as COVID-19.

### Example 5: Phase 2 Clinical Trial

The following clinical trial may be performed using a niclosamide composition described herein, such as the 5% NEN formulations defined in Example 1, or the formulation described in Table 8 of Example 6.

### Clinical Trial Protocol

This study is to assess the safety and efficacy of a formulation of the invention in subjects with mild to moderate COVID-19. Its primary endpoint is Time to clinical improvement (defined as at least 2 grades improvement in the modified WHO ordinal scale). This trial has an adaptive design and includes two intermediate analyses: 1. a safety analysis based on the data collected in the first 20 patients enrolled and hospitalized; 2. A proof of mechanism based on the antiviral activity measured by viral load in the first 80 subjects treated. These analyses will be conducted by a SMC which will recommend on two decisions: the authorization to treat patients at home and the decision to complete the study.

The phase II study will focus on the population that is likely to respond to a drug with a primary mode of action being to prevent viral replication: patients hospitalized with moderate COVID-19 and subjects with flu-like signs and symptoms not needing hospitalization (mild COVID-19).

All enrolled subjects will receive a formulation of the invention or a placebo in a twice-daily procedure including a spray of 150 μL of the investigational product in each nostril followed by the nebulization of 6 mL of the investigational product. The treatment duration is 14 days for all subjects, even in case of clinical cure. In patients showing a worsening of the signs and symptoms of COVID-19, treatment should be pursued without change, unless an exclusion criterion would be met, for example need for mechanical ventilation or hospitalization in an intensive care unit.

For confirming the safety of a formulation of the invention, the 20 first subjects included in this study will be hospitalized during the first days of treatment (hospitalization could be prolonged at investigator discretion and depending on the respiratory or medical status). A SMC would analyze all safety data generated in these subjects and recommend on the safety of administering the treatment at home by a nurse in subjects not needing hospitalization.

After 80 subjects will have completed the study (this number is subject to revision following Statistical input), a soft database lock will be performed for these subjects and an analysis will be conducted to provide proof of mechanism by confirming efficacy on the anti-viral end points of the formulation of the invention, based on the time to viral clearance (measured via throat swabs or saliva sampling, the most sensitive and specific test being to be confirmed). The DMC will review the data of this analysis, and should they find no anti-viral effect, the DMC could recommend stopping the study for futility. While the proof of mechanism analysis is ongoing recruitment of the remaining subjects will continue.

### Study Population

Eligible subjects for this study must have a positive test confirming infection with SARS-CoV-2 and present signs and symptoms of COVID-19. They cannot currently be treated with other anti-viral treatments or other investigational products. Standard of care treatments are allowed and should be recorded as concomitant treatments. Patients with severe and unstable concomitant pathologies, patients needing invasive mechanical ventilation or extracorporeal membrane oxygenation and patients hospitalized in intensive care units cannot be enrolled.

### Product Administration

In this study, the investigational product will be administered by a qualified person at home or in the centre where the subject would have been admitted for isolation or at the hospital. The qualified person is either a physician, a medical student or a nurse specifically trained with the product and its potential risks.

### Efficacy Assessments

The anti-viral efficacy will be assessed by the SARS-CoV-2 titers determined by PT-PCR from saliva or nasopharyngeal samples collected at baseline and every day until D14 (most sensitive and specific test still to be confirmed).

The Clinical efficacy in all subjects will be based on the assessment of flu-like symptoms scoring (by investigator and patient), oximetry, NEWS2 score and COVID-19 severity based on an ordinal scale. The ordinal scale is derived from the scale for clinical improvement defined by the WHO committee and used in the remdesivir studies. This scale had however to be adapted to capture milder severities.

The NEWS2 score is based on a simple aggregate scoring system in which a score is allocated to physiological measurements, already recorded in routine practice, when patients present to, or are being monitored in hospital. Six simple physiological parameters form the basis of the scoring system: respiration rate, oxygen saturation, systolic blood pressure, pulse rate, level of consciousness, temperature (see below).

In addition, metrics defined in the FDA guidance will be used:
All-cause mortality
Respiratory failure (i.e., need for mechanical ventilation, ECMO, noninvasive ventilation, or high-flow nasal cannula oxygen delivery)
Need for intensive care unit (ICU) level care based on clear definitions and specific clinical criteria
Need for hospitalization
Sustained clinical recovery (e.g., resolution of symptoms)—Chest x-ray (or other imaging, e.g., CT scan) and serum inflammatory biomarkers (primary marker: CRP) will be considered exploratory measures.

Functional respiratory tests cannot be conducted acutely during the active phase of viral infection. These tests will however be conducted after viral clearance in a specialized respiratory unit, two weeks and one months after treatment discontinuation if the patient condition allows it.

### Number of Subjects

This study will enroll approximately 350 subjects (the exact number may be determined following statistical input) to ensure a good representation of the different levels of disease severity of mild to moderate COVID-19. The recruitment of a sufficient number of subjects with either mild or moderate COVID-19 will be secured by a careful selection of study centres and involvement of hospital units.

The sample size needed for the interim analysis is based on the assumption that a clinically relevant and medically meaningful benefit is defined by at least 4-day difference in the meantime to viral clearance (defined as first day of 2 consecutive negative tests) when compared to placebo.

### Inclusion Criteria

Subjects are only eligible if they fulfil all criteria for inclusion:

1. Age ≤18 and <80 years
2. Male or nonpregnant and nonlactating female who is abstinent throughout the course of the study. Females must have a negative urine beta-human chorionic gonadotropin hormone (hCG) pregnancy test at Day 1. (Women who are postmenopausal or who had tubal ligation/hysterectomy do not need to have a urine or serum pregnancy test and do not need to agree to use contraception.)
3. Able to understand and provide signed informed consent.
4. With Nasopharyngeal swab or saliva test confirming infection with SARS-CoV-2 and mild to moderate signs and symptoms of COVID-19.

### Exclusion Criteria

Subjects who meet any of the following criteria are not eligible to participate in this study:

1. Enrolment in a niclosamide study in the previous 6 months

2. Allergy to niclosamide or history of significant adverse reaction to niclosamide or related compounds, or to any of the excipients used.

3. Underlying condition that may interfere with inhalation of the IP.

4. Current acute or chronic unstable condition (incl. respiratory disease, CV disease, diabetes mellitus, obesity) unless considered clinically irrelevant by the investigator.

5. The presence of a condition the investigator believes would interfere with the ability to provide consent, or comply with study instructions, or that might confound the interpretation of the study results or put the subject at undue risk.

6. Active or acute infection other than SARS-CoV-2, including bacterial superinfection.

7. Severe COVID-19 requiring mechanical ventilation or admission in an intensive care unit.

### Prior or Concomitant Therapy

8. Systemic anti-viral therapies or other investigational products the month prior Day 1.

9. Anti-cancer or immunosuppressive drugs three months prior to Day 1.

### Test Product(s), Dose, Mode of Administration, and Duration of Treatment

A nurse, a dedicated medical student, or an investigator will administer 150 μL of a formulation of the invention or placebo in each nostril followed by a nebulization of 6 mL of the formulation of the invention or placebo, twice daily for a maximum of 14 days.

### Duration of Study

Subject participation in the study (excluding the screening period) is 14 days with an additional 14-day follow-up period to be extended to 28-days if respiratory function is still abnormal.

### Efficacy Variables

Anti-viral Efficacy will be assessed based on:
Eradication of SARS-CoV-2 (in the nasopharynx)
Viral load

Clinical Efficacy will be assessed based on:
Changes in the modified WHO ordinal scale
Severity of Flu-like signs and symptoms (including fever)
Changes in Respiratory status assessed by oximetry (blood oxygen saturation)
Changes in NEWS2 score
Chest x-ray (or other imaging, e.g., CT scans) abnormalities
Change in serum inflammatory biomarkers (primary marker: CRP)
Respiratory function status at end of follow-up period
Shortness of breath questionnaire with St George Respiratory Questionnaire
Specific respiratory tests will be conducted to assess of the status pulmonary function.

### PK Variables

A blood sample will be collected on Day 7 and at D14 for measure of trough levels before investigational product administration.

### Endpoints and Criteria for Evaluation

Primary Endpoint

Time to clinical improvement (at least 2 grades in the modified WHO ordinal scale).

### Secondary Endpoints

Based on modified WHO ordinal scale:
Percentage of subject cleared (score 0) at D7 and D14
Percentage of live subjects discharged from hospital at D7, D14 and FU visits
Percentage of subjects with score 6 at D7, D14 and FU visits
Distribution within different scores at D7, D14 and FU visits
Percentage of subjects having worsened by 1, 2, 3 or more grades
Based on viral sampling:
Time to viral clearance defined as the time to the first of two consecutive negative tests for SARS-CoV-2
Percentage of subjects achieving viral clearance at each visit
Mean viral load (AUC of viral particle titers) during the 14-day period a Mean peak viral load during the 14-day period
Based on the Flu-like scores:
Mean and worst severity score of Flu-like signs and symptoms
Time to disappearance of Flu-like signs and symptoms
Based on Oximetry:
Percentage of subjects needing oxygen supply at D7 and D14
Mean and worst oximetry measure
Time to independence from oxygen therapy
Based on NEWS2 score:
Mean and worst NEWS2 score
Based on SGRQ:
Mean score and worst score
Based on respiratory tests (VO2max, DCO):
Share of subjects with normal function at FU visits
Mean reduction compared to predicted normal function at FU visits
Percentage of subjects with pulmonary edema/inflammation as assessed by chest x-ray (or other imaging, e.g., CT scan)
Mean change in Inflammatory serum biomarker (CRP, Procalcitonin)

These endpoints will be calculated for the overall population as well as for 2 subpopulations (Mild and Moderate COVID-19 as assessed at baseline).

### Scales and Scores

### Flu-Like Symptoms Scales

Physician or nurse assessment includes 14 cardinal signs and symptoms to be scored on a 4-point scale from 0 (none) to 3 (Severe) for a total score ranging from 0 to 42:

chills

fever

muscle pain

fatigue

cough

shortness of breath

sneezing

loss of appetite

headache

nasal congestion

ear pain

nausea, vomiting

loss of odor or taste

wheezing.

Patient self-assessment is done using FLU-PRO (Powers et al., Performance of the inFLUenza Patient-Reported Outcome (FLU-PRO) diaryin patients with influenza-like illness (ILI). PLoS One. 2018; 13(3):e0194180) or FLUIIQ (Osborne et al., Development and validation of the Influenza Intensity and Impact Questionnaire (FluiiQ™). Value Health. 2011; 14(5):687-699).

TABLE 4
Modified WHO ordinal scale

| Severity | Descriptor | Score |
|---|---|---|
| No disease | Uninfected, no clinical or virological evidence of infection | 0 |
| Healthy | Positive testing by reverse transcription polymerase chain reaction | 1 |
| carriers | (RT-PCR) assay or equivalent test for SARS-CoV-2, no symptoms | |
| Very mild | Ambulatory. Virological evidence of SARS-CoV-2. Symptoms of mild | 2 |
| disease | illness with COVID-19 that could include fever, cough, sore throat, | |
| | malaise, headache, muscle pain, gastrointestinal symptoms. | |
| | No significant limitation of activities. No signs of viral pneumonia like | |
| | shortness of breath of dyspnea. | |
| Mild disease - | Ambulatory. Virological evidence of SARS-CoV-2. Symptoms of mild | 3 |
| ambulatory | illness with COVID-19 that could include fever, cough, sore throat, | |
| | malaise, headache, muscle pain, gastrointestinal symptoms. | |
| | Limitation of activities. | |
| | No or minimal signs of viral pneumonia which do not require special | |
| | medical care, supplemental oxygen or hospitalization. | |
| Mild disease - | Hospitalized. Virological evidence of SARS-CoV-2. | 4 |
| hospitalized | Regardless the severity of the Flu-like syndrome, mild signs of | |
| | pneumonia which require medical attention. | |
| | No supplemental oxygen needed. | |
| Moderate | Hospitalized. Virological evidence of SARS-CoV-2. | 5 |
| disease | Regardless the severity of the Flu-like syndrome, moderate viral | |
| | pneumonia requiring oxygen by mask or nasal prongs. | |
| Severe | Hospitalized. Virological evidence of SARS-CoV-2. | 6 |
| disease | Severe viral pneumonia requiring high-flow oxygen or non-invasive | |
| | positive pressure ventilation. | |
| Very severe | Hospitalized. Virological evidence of SARS-CoV-2. | 7 |
| disease | Very severe viral pneumonia requiring intubation and mechanical | |
| | ventilation. | |
| Critical | Virological evidence of SARS-CoV-2. Hospitalized with critical | 8 |
| disease | disease necessitating ventilation + additional organ support - | |
| | pressors, RRT, ECMO. | |
| Fatal disease | Virological evidence of SARS-CoV-2. Death. | 9 |

TABLE 5
NEWS2 score

| Physiological | Score | | | | | | |
|---|---|---|---|---|---|---|---|
| parameter | 3 | 2 | 1 | 0 | 1 | 2 | 3 |
| Respiration rate | ≤8 | | 9-11 | 12-20 | | 21-24 | ≥25 |
| (per minute) | | | | | | | |
| SpO₂ Scale 1 (%) | ≤91 | 92-93 | 94-95 | ≥96 | | | |
| SpO₂ Scale 2 (%) | ≤83 | 84-85 | 86-87 | 88-92 | 93-94 on | 95-96 on | ≥97 on |
| | | | | | oxygen | oxygen | oxygen |
| Air or oxygen | | Oxygen | | Air | | | |
| Systolic blood | ≤90 | 91-100 | 101-110 | 111-219 | | | ≥220 |
| pressure (mmHg) | | | | | | | |
| Pulse (per minute) | ≤40 | | 41-50 | 51-90 | 91-110 | 111-130 | ≥131 |
| Consciousness | | | | Alert | | | CVPU |
| Temperature (° C.) | ≤35.0 | | 35.1-36.0 | 36.1-38.0 | 38.1-39.0 | ≥39.1 | |

TABLE 6
NEWS thresholds and triggers

| NEW score | Clinical risk | Response |
|---|---|---|
| Aggregate score 0-4 | Low | Ward-based response |
| Score of 3 in any individual | Low-medium | urgent ward-based |
| parameter | | response |
| Aggregate score of 5-6 | Medium | Key threshold for urgent |
| | | response |
| Aggregate score of 7 or more | High | Urgent or emergency |
| | | response |

### Example 6 Manufacturing of Niclosamide Ethanolamine Solution

### Aqueous Formulation Comprising Niclosamide Ethanolamine

The formulation shown in Table 7A was prepared as described below:

TABLE 7A

| | Composition |
|---|---|
| | Formulation A |
| Component (trade name) | % (w/v) |
| Niclosamide ethanolamine | 1.0 |
| Polyvinylpyrrolidone | 2.0 |
| (PVP K30) | |
| Hydroxypropyl Beta-cyclodextrin | 15.0 |
| (Kleptose ® HPB) | |
| DMSO | 1.0 |
| 1M NaOH/HCL | As required to adjust pH to |
| | 7.8 |
| Water (milliQ) | q.s. |

Niclosamide ethanolamine (100 mg), PVP (K30) (200 mg) and hydroxypropyl beta-cyclodextrin (1500 mg) were weighed into a 20 mL glass vial. To this powder mixture was added milliQ water (8.5 mL), 2 drops 5M NaOH, and 100 μL DMSO. The pH of the mixture was at least 8. The vial was placed in an ultrasonic bath at 65° C. and mixed according to the following schedule:

Vortex for 1 to 10 minutes; ultrasonication at 65° C.; vortex for 1 to 5 minutes;
ultrasonication at 65° C.; and vortex 1 min;
to provide a clear red composition without any visible particles.
2 drops 5M HCl were added to the composition followed by vortexing for 1 minute. The resulting formulation was cooled to room temperature and the pH adjusted to 7.80±0.1 using 1 M NaOH/HCl to give the title formulation. The Osmolarity of the title composition was 180 mOsm/kg.

Formulations B to E shown in Table 7B were prepared using an analogous method.

TABLE 7B

| | Composition | | | |
|---|---|---|---|---|
| | B | C | D | E |
| Component (trade name) | % (w/v) | % (w/v) | % (w/v) | % (w/v) |
| Niclosamide | 1 | 1 | 1 | 2 |
| ethanolamine | | | | |
| Polyvinylpyrrolidone | — | 2 | 2 | 2 |
| (PVP K30) | | | | |
| Polyvinylpyrrolidone | 2 | — | — | — |
| (PVP K17) | | | | |
| Hydroxypropyl Beta- | 15 | 15 | 50 | 50 |
| cyclodextrin | | | | |
| (Kleptose ® HPB) | | | | |
| DMSO | — | — | — | — |
| 1M NaOH/HCL | As | As | As | As |
| | required to | required to | required to | required to |
| | adjust pH | adjust pH | adjust pH | adjust pH |
| | to 7.5-7.8 | to 7.5-7.8 | to 7.5-7.8 | to 7.5-7.8 |
| Water (milliQ) | q.s. | q.s. | q.s. | q.s. |

### Batch Manufacturing of Niclosamide Ethanolamine Nebulizer Solution

The formulation shown in Table 8 was prepared as described below:

TABLE 8

| | Composition |
|---|---|
| | Formulation F |
| Component (trade name) | % (w/w) |
| Niclosamide ethanolamine | 1.0 |
| Polyvinylpyrrolidone (PVP K30) | 2.0 |
| Hydroxypropyl Beta-cyclodextrin (Kleptose ® HPB) | 15.00 |
| NaOH | ca. 0.2* |
| 2N HCl | ca. 2.0* |
| Water for injection | q.s. up to |
| Total | 100.00 |

*as required to provide a pH of about 7.8

The nebuliser solution 1% is an isotonic and euhydric aqueous formulation. The solution was filled into 10 mL clear type I moulded glass vials, each vial containing 7 mL of the solution. The nebuliser solution 1% contains 10 mg/mL niclosamide ethanolamine, equivalent to 8.4 mg/mL of niclosamide free base.

The batch formula for 10 kg nebuliser solution 1% is shown in Table 9:

TABLE 9

| Ingredient | Quantity (kg) |
|---|---|
| Niclosamide ethanolamine | 0.10 |
| Polyvinylpyrrolidone (PVP K30) | 0.20 |
| Hydroxypropyl Beta-cyclodextrin (Kleptose ® HPB) | 1.50 |
| NaOH | ca. 0.02* |
| 2N HCl | ca. 0.20* |
| Water for injection | q.s. up to |
| Total | 10.00 |

*as required to provide a final pH of about 7.8

The bulk solution was prepared in a class C environment according to the following protocol:

1. A tank was charged with hot water (e.g. 65-90° C.) for injection (80% of the total quantity) and stirring was started;
2. The tank was charged with cyclodextrin and NaOH and the mixture stirred until the solid components were completely dissolved to provide a solution of about pH 12;
3. Solid niclosamide ethanolamine was added to the tank and stirring was continued until the niclosamide ethanolamine was completely dissolved to give a solution of approximately pH 8-9;
4. 75% of the total of 2N HCl was added;
5. PVP was added and stirring continued until the PVP was completely dissolved;
6. The solution was cooled to about room temperature;
7. The pH of the solution was adjusted to 7.8 by addition of the remaining 2N HCl, and the pH was recorded;
8. Water for injection was added to the final weight;
9. The solution was discharged into 10 mL glass vials (7 mL solution per vial);
10. The vials were closed with a rubber stopper and sealed with an aluminium cap.

The results of analysis of batches manufactured according to the above protocol are shown below in Table 10:

TABLE 10

| Test | Limit | Batch 1 | Batch 2 |
|---|---|---|---|
| Appearance | Clear red-orange | Complies | Complies |
| | solution essentially | | |
| | free of visible | | |
| | particles | | |
| Identity Niclosamide | positive (HPLC, | positive | positive |
| Ethanolamine | conforms with | | |
| | retention time of | | |
| | reference) | | |
| Assay Niclosamide | 0.90-1.10 (w/w) | 0.97% | 0.98% |
| Ethanolamine | | | |
| Related substances | | | |
| Specified identified: | | | |
| 5-chlorosalicylic acid | <0.3% (w/w) of DS | n.d. | <0.15% |
| 2-chloro-4-nitroaniline | <0.5% (w/w) of DS | n.d. | <0.25% |
| Single unknown impurity | <0.5% (rel. | n.d. | <0.25% |
| | area %) of DS | | |
| Total Impurities | <2.0% (rel. | n.d. | <1.0% |
| | area %) of DS | | |
| Minimum Fill Volume | 7.0 mL | 7.0 mL | 6.7 mL |
| pH | 7.6-8.0 | 7.9 | 8.0 |
| Osmolality | 290-320 mosm | NA | 315 |
| Microbial Quality | TAMC <102 cfu/g | <100 | <100 |
| | TYMC <101 cfu/g | <10 | <10 |
| | S. aureus: absent | Complies | Complies |
| | P. aeruginosa: | Complies | Complies |
| | absent | | |
| | Bile-tolerant gram- | Complies | Complies |
| | negative bacteria: | | |
| | absent | | |

### Example 7: Non-Clinical Studies

### Study A: Dose Range Finding and 2 Week GLP Inhalation Toxicity Study in the Rat

The objectives of this study were to determine the potential toxicity of the formulation shown in Table 8 of Example 6 (1% niclosamide ethanolamine, 2% PVP K30 and 15% Kleptose HPB), when given by inhalation administration to rats at escalating dose level to determine a maximum tolerated dose (MTD Phase) followed by a 2 week repeat dose phase (Fixed Dose Phase) and to evaluate the potential reversibility of any findings. In addition, the toxicokinetic characteristics of Formulation A were determined.

The pivotal 2-week safety study in rats was assessed using daily dose levels of 15 (5-fold higher [systemic mg/kg] and 18-fold higher [local mg/g] compared to a human 30 mg, qd dose) and 50 mg/kg (15-fold higher [systemic mg/kg] and 52-fold higher [local mg/g] compared to a human 30 mg, qd dose) (10 rats/sex/group for main study evaluation); both vehicle and air control groups were also included. This pivotal phase was preceded by a range finding phase which selected a high dose level of 50 mg/kg for use in the pivotal 2-week phase. Microscopic evaluation of the nasal cavity in rats after 2 weeks of daily dosing revealed a non-adverse minimal hypertrophy of goblet (mucin-secreting) cells in the nasal septum/nasopharynx at 15 and 50 mg/kg which was not dose related; these changes were not observed in the vehicle or air control groups and were considered an adaptive change to repeated administration of niclosamide ethanolamine. In the lungs, minimal to mild increase in alveolar macrophages were observed after 2 weeks of dosing in the vehicle and 15 and 50 mg/kg dose groups; these changes were not considered adverse but instead an adaptive to response to clearance of the vehicle. No other noteworthy histopathological findings have been reported to date.

### Study B: Dose Range Finding and 2 Week GLP Inhalation Toxicity Study in the Beagle Dog

The objectives of this study were to determine the potential toxicity of the formulation shown in Table 8 of Example 6, when given by inhalation administration to dogs at escalating dose level to determine a maximum tolerated dose (MTD Phase) followed by a 2 week repeat dose phase (Fixed Dose Phase) and to evaluate the potential reversibility of any findings. In addition, the toxicokinetic characteristics of the formulation were determined.

The pivotal 2-week safety study in dogs was assessed using daily dose levels of 2.5 (2-fold [systemic mg/kg] and 4-fold higher [local mg/g] to a human 30 mg, qd dose) and 4.37/4.14 mg/kg (3-fold higher [systemic mg/kg] and 6-fold higher [local mg/g] compared to a human 30 mg, qd dose) (3/sex/group for main study evaluation); both vehicle and air control groups were also included. After 2 weeks of daily dosing, microscopic evaluation in male and female dogs administered vehicle or 2.5 mg/kg and in females dosed at 4.14 mg/kg revealed no changes to the nasal cavity and only minimal changes in the lungs including minimal increased alveolar macrophages and mixed/mononuclear cell infiltration, minimal bronchial exudate, and mild neutrophilic infiltration or mild increased cellularity in tracheobronchial lymph node secondary to the minimal lung findings. The histological changes seen after 2 weeks of dosing were minor and not considered adverse.

### Study C: Pulmonary Pharmacokinetics of Nebulized Niclosamide in Sheep Following Pulmonary Administration (Non-GLP)

The objectives of this study were to determine the pharmacokinetic profile of the formulation shown in Table 8 of Example 6, when given by pulmonary administration to sheep at escalating dose levels similar to the clinical escalation scheme coupled with a safety assessment using lung function tests.

The PK analysis in the sheep following treatment demonstrated substantial exposure of niclosamide in the epithelial lining fluid (ELF). Peak concentrations exceed 100-fold of the IC90 value of niclosamide against SARS-CoV-2. In spite of substantial clearance from the ELF, niclosamide concentrations above the IC90 are maintained for the 8-hour sampling period following a single administration Formulation A (FIG. 1A). These data support the twice daily administration of the formulation.

Additionally, the ELF concentrations of niclosamide in this study greatly exceed the published plasma pharmacokinetics published from studies using oral niclosamide and provide the pharmacological rationale for using formulations according to the invention for treatment of COVID-19 compared to oral dosage forms of niclosamide. As viral elimination is most likely driven by pulmonary rather than systemic exposure, the efficacy margin achieved with Formulation A following pulmonary administration is much greater (efficacy margin of mean Cmax in ELF to IC90 is >100 fold) in the relevant region of viral replication than the one with the oral route (efficacy margin mean systemic exposure of human oral dose to IC90 exists only for 2 g/day dose, which is 8-fold), although definite lung levels after oral administration of niclosamide remain unknown (FIG. 1B).

Niclosamide systemic exposure after administration was in the range of values reported in humans following oral exposure, with Cₘₐₓ of 577 ng/mL (mean) [range: 217-803 ng/mL]. Additionally, the treatment was found to be well tolerated in the sheep as determined by lung function analysis pre and post dosing.

### Example 8: Phase I Trial of Inhaled Niclosamide

A randomized, placebo-controlled, double-blind, multiple dosing Phase 1 trial was conducted to assess the safety of the formulation shown in Table 8 of Example 6 (or an equivalent formulation comprising 1% w/w niclosamide, the balance being water) in healthy volunteers.

### Methods

### Trial Design and Oversight

This was a single centre, interventional, double-blinded (open label for the first sentinel subject within each cohort), placebo-controlled, Phase 1 study to assess the safety and explore PK parameters of niclosamide ethanolamine in healthy volunteers (HV). The study consisted of five cohorts, which started one after the other, each after consultation of the Safety Monitoring Committee (SMC). Each cohort started only if the previously collected data did not give raise to safety concerns. 44 eligible HVs were enrolled in five sequential cohorts for dose finding, each cohort were screened generally followed by extended respiratory work-out one or two days before dosing. If all inclusion and no exclusion criteria were met, dosing was fulfilled followed by 24 hours monitoring. After 48 hours, all participants had the same extensive respiratory work-out as prior to the study inclusion. 34 of these 44 healthy controls receive the investigational product (IP), and 10 the placebo. The study was partly conducted in an open-label design (first subject in cohort 1-4 as sentinel subject), and partly double blinded (subsequent subjects in cohorts 1-4 and all subjects in cohort 5). The doses of the different cohorts are displayed in Table 11.

TABLE 11
Summary of cohorts with its dose and duration of treatment

| Cohort | Dose |
|---|---|
| 1 | 9 healthy volunteers, 7 received a single dose of formulation (4 mL, 0.1%, equalling |
| | 3.4 mg niclosamide) and nasal spray (2 × 150 μL, 0.1%, i.e. once per nostril, |
| | totalling 0.25 mg niclosamide) and 2 received placebo |
| 2 | 9 healthy volunteers, 7 received a single dose of formulation (1 mL, 1%, equalling |
| | 8.4 mg niclosamide) and nasal spray (2 × 150 μL, 1%, totalling 0.25 mg |
| | niclosamide) and 2 received placebo. |
| 3 | 9 healthy volunteers, 7 received a single dose of formulation (3 mL, 1%, equalling |
| | 25.2 mg niclosamide) and nasal spray (2 × 150 μL, 1%, totalling 0.25 mg |
| | niclosamide) and 2 received placebo. |
| 4 | 9 healthy volunteers, 7 received a single dose of formulation (6 mL, 1%, equalling |
| | 50.4 mg niclosamide) and nasal spray (2 × 150 μL, 1%, totalling 0.25 mg |
| | niclosamide) and 2 received placebo. |
| 5 | 8 healthy volunteers, 6 received five doses of formulation (6 mL, 1%, equalling 50.4 |
| | mg niclosamide per dosing and 252 mg in total) and nasal spray (2 × 150 μL, 1%, |
| | totalling 2.5 mg niclosamide per dosing and 12.6 mg in total) dosed BID for 2.5 |
| | days and 2 received placebo. |

For cohorts 1-4, one subject was dosed with the IP the first day (Monday) and followed for 24 hours while admitted at the clinic to assess safety of the new dose. Safety visit with extended lung function measurements were performed the following Wednesday to Friday at CFAS. For cohort 5, patients received a total of 5 administrations and stayed at the trial site for 3 days (Monday or Tuesday onto Thursday or Friday), including overnight. In cohort 5, as the dose was the same as in cohort 4, all patients were blinded and randomized. Safety visit with extended lung function measurements were performed the following Thursday to Saturday at CFAS.

Throughout the study, both IP were administered by qualified study staff. Each treatment was assigned to a specific subject by randomization number. Screening and enrolment was done sequentially for one cohort after the other. A randomization number was assigned in ascending order to each eligible subject at Day 0 according to the randomization list by cohort. The first number of the cohorts 1, 2, 3 and 4 was always active (open label) and the remaining consisted of 6 active and 2 placebos (n=9). For cohort 5, the numbers consisted of 6 active and 2 placebos (n=8).

### Eligibility

Subjects who signed Informed Consent Form (ICF), were male or nonpregnant and nonlactating female who was abstinent or agreed to use effective contraceptive methods throughout the course of the study, females who had a negative urine beta-human chorionic gonadotropin hormone (hCG) pregnancy test prior to and did not need to agree to use contraception, showed an electrocardiogram (ECG) without clinically significant abnormalities (including QTcF<450 ms), were ≥18 and <65 years at the time of signing ICF, were normally active and in good health by medical history with no current chronic diseases and normal physical examination, had minimum 80% of predicted lung function, including expiratory volume (FEV1) after β2-agonist, static volume (TLC), diffusion capacity (DCO), and normal cardiopulmonary exercise testing (CPET) with pulse oximetry as well as ECG with a fitness score of >20 mLO₂/kg*min for females and >25 mLO₂/kg*min and no clinical important arrythmia or desaturation during exercise and furthermore, showed a chest X-ray without clinically significant abnormalities were eligible to participate in this study. Subject that had clinically significant allergies, current acute or chronic condition, renal impairment, underlying condition that may interfere with inhalation of IP, and consumed alcohol in the 24 hours prior dosing were excluded.

### Safety Assessment and Outcome Measures

Safety was assessed through the following parameters: adverse events (AEs) reporting, general safety assessments, general physical examination, vital signs, clinical laboratory analysis, including urinalysis, haematology, and serum chemistry, ECGs, vital capacity, TLC, DCO, FEV1, reversibility, fraction of expiratory nitric oxide (FeNO) tests, resting pulse oximetry and CPET with ECG and pulse oximetry.

The primary endpoint was defined as the AE frequency in each cohort and treatment group and the change from baseline for all safety variables measured and frequency of out of range values. Furthermore, the pharmacokinetics following administration was evaluated by determining the maximum concentration of active drug molecules in blood (Cₘₐₓ), time to reach maximum level (Tmax), area under the curve of drug level in blood versus time (AUC) and the half-life (T½).

### Primary Endpoints

AE frequency in each cohort and treatment group
Change from baseline for all safety variables measured and frequency of out of range values
In addition to AEs/SAEs collection throughout the study duration, general safety will be assessed via clinical examination, vital sign assessments, ECGs, and laboratory analysis (serum chemistry, hematology, and urinalysis).
Pulmonary function monitored by measurement of vital capacity, expiratory volume (Forced Respiratory Volume in one second, FEV1), static volume (Total Lung Capacity, TLC), diffusion capacity (DCO), exhaled nitric oxide (FeNO) and resting pulse oximetry.

### Secondary Endpoints—PK

Maximum concentration of active drug molecules in blood (Cₘₐₓ)
Time to reach maximum level (Tₘₐₓ)
Area Under the Curve of drug level in blood versus time (AUC)
Half life

### Statistical Analysis

The sample size was considered sufficient to meet the study objectives and to assess treatment safety but was not based on statistical power considerations. Two sets of populations for analysis were distinguished, the Safety Set and the PK Set. The Safety Analysis Set includes data from all enrolled subjects receiving any amount of IP. Descriptive statistics are reported for continuous variables and metric values, including the number of subjects, mean (μ), standard deviation (SD), median, minimum (Min), and maximum (Max). Categorical variables are reported as frequencies and percentages. For metric values, absolute change of since baseline are reported, except for FEV1 percentage change is shown. Significance of differences was tested in an exploratory fashion. No imputation for missing data was made. Data from patients receiving placebo were combined across cohorts. For all analyses, the statistical software Stata® (version 16) was used in the most recent sub-version available at data base lock.

The PK Analysis Set included data from subjects who were treated and have no missing data affecting the PK assessment. Subjects with at least one quantifiable drug concentration were included in the PK analysis. No imputation for missing data was made. All pharmacokinetic parameters were calculated using non-compartmental analysis (NCA) with a validated installation of the software Phoenix® WinNonlin® version 8.1.

### Results

### Trial Population

Forty-four subjects were randomized of which 34 were assigned to treatment and 10 to placebo.

### Safety Outcomes

No serious AE nor early discontinuation was reported in this study. In total, 32 subjects experienced one or more AEs during the study. The majority of the AEs belonged to the “Respiratory, thoracic, and mediastinal disorders” category with “Upper respiratory tract irritation” being the most frequent AE descriptor (45 events in 26 subjects, 59%) and corresponding to throat irritation during and after nebulization. Furthermore, the nasal applications did not result in any finding with regards to local tolerability. For the nebulization procedure, there was a dose-dependent difference in terms of tolerability. However, all AEs reported were mild and disappeared spontaneously and completely without treatment in one to two hours. For most subjects, symptoms were more pronounced during the first 5-10 minutes of the inhalation procedure. Of note, in the multiple administration group, most subjects reported that symptoms decreased over time with repeated dosing. During administration of the drug, some subjects showed an asymptomatic, but significant decrease in FEV1 (>200 mL and >12%), which was reversible with a beta2 agonist, whereas none of the subjects experienced decrease in FVC, nor in DCO.

Asymptomatic airway obstruction (decline in FEV1) was shown in 4 subjects, 3 out of 4 occurring in the highest dose (6 mL) group. These events were all responsive to inhaled β2-mimetic treatment.

In cohort 5, the mean (SD) oxygen uptake was unchanged 3401 (551) prior to drug administration and 3359 (516) (NS), and the mean workload was similar at the two measurements 309 (56) versus 300 (54), NS). Likewise, the FEV1 post beta2-agonist was 116 (16) pre-drug values, and post drug administration 111 (17) (NS), and FVC 117 (14) and 114 (13), respectively (NS) and TLC 104 (11) and 104 (10), respectively (NS). DCO was found to have a significant decrease 102 (10) versus 90 (6), p=0.01, however none showed a clinically significant change of more than 20%. Post drug safety lung function measurements showed asymptomatic decrease in post beta2-agonist FEV1 measurement 1 participants (from 124% pred to 108% pred), two developed significant reversibility (18% and 12%), and 4 had signs of increased airway inflammation (identified as a change in fractional nitric oxide concentration in exhaled breath [FENO]) (Change of 11 ppb, 37 ppb, 37 ppb, 28 ppb) of whom one had elevated FeNO prior to drug administration, all in Cohort 5. None showed clinically significant change in TLC, or VO₂max in cohort 1 to 5. One showed a decrease in DCO in cohort 3 (15%) and 3 in cohort 5 (19%, 18%, 16%), however KCO was in all cases unchanged within the clinical acceptable limit.

All but one of the AEs related to abnormal test values were reported with the highest dose in either cohort 4 or cohort 5 and all these events were reported in the active groups and considered by the investigator as being possibly, probably or definitely related to the test product or procedure.

### Pharmacokinetics

Pharmacokinetic analyses demonstrated dose-proportional characteristics for niclosamide ethanolamine (FIG. 2). The maximum plasma concentration (Cₘₐₓ) and Area under the curve (AUC₀₋₃) levels following a single dose application were 238.9 ng/mL (mean) and 509.0 hr*ng/mL (mean). Following repeated dosing in Cohort 5, Cₘₐₓ and AUC₀₋₈ levels of 337.3 ng/mL and 401.2 hr*ng/mL were reported, indicating no accumulation of niclosamide ethanolamine after repeated dosing.

Raw data indicated peak concentrations in blood of 337 ng/mL (mean) [range: 29-506 ng/mL] after repeated inhalation doses. The half-life was shown to be 2 hours (mean) in cohort 4 and 2.7 hours (mean) in cohort 5. This is in the range of systemic exposure reported after oral dosing of niclosamide (see FIG. 3). The systemic PK data from humans (including dose response) is in close agreement with the data from the sheep PK study (see FIG. 4).

As a preliminary conclusion, the formulation of the invention appears to provide systemic exposure within the range observed with the approved 2 g oral dosage form of niclosamide (Yomessan). Additionally, given the route of administration and the sheep ELF PK data, the concentration in the lungs is substantially higher than oral niclosamide and accordingly the formulation would represent a preferred treatment of COVID19 compared to oral dosage forms of niclosamide.

### Example 9: Phase 3 Clinical Trial

This is a randomized, parallel-group, placebo-controlled, blinded, multi-center, phase 3 treatment study to assess the safety and efficacy of a 1% niclosamide ethanolamine solution (administered via a nebulizer and as a nasal spray) in hospitalized participants with mild and moderate COVID-19. The dose chosen for this study is twice daily inhalation of 3 mL, 1% niclosamide ethanolamine (equaling 27.4 mg niclosamide) and 150 μL nasal spray, 1%, once per nostril (totaling 2.6 mg niclosamide).

### Study Population

In the context of this study, mild and moderate COVID-19 infection is defined by the FDA guidance as follows:

### Mild:

Positive testing by standard RT-PCR assay or equivalent testing
Symptoms of mild illness could include fever, cough, sore throat, malaise, headache, muscle pain, GI symptoms, without shortness of breath or dyspnea
No clinical signs indicative of Moderate, Severe, or Critical severity

### Moderate:

Positive testing by standard RT-PCR assay or equivalent testing
Symptoms of moderate illness could include any symptom of mild illness or shortness of breath with exertion
Clinical signs suggestive of moderate illness with COVID-19, such as respiratory rate ≥20 breaths/minute, SpO₂>93% on room air at sea level, heart rate ≥90 beats/minute
No clinical signs indicative of Severe or Critical severity

### Inclusion Criteria

Participants are eligible to be included in the study only if all of the following criteria apply:

1. Participant must be 18 years of age at the time of signing the informed consent form (ICF)
2. Fulfill the criteria for mild to moderate signs and symptoms of COVID-19 as defined by FDA guidance
3. Symptoms or signs of COVID-19 for no more than 4 days prior to enrolment
4. Have test confirming infection with SARS-CoV-2
5. Currently hospitalized
6. Male or nonpregnant and nonlactating female who is abstinent or using contraception throughout the study. Females must have a negative urine beta-human chorionic gonadotropin hormone pregnancy test at Day 1. (Women who are postmenopausal or who had tubal ligation/hysterectomy do not need to have a pregnancy test and do not need to agree to use contraception.)
7. Capable of giving signed informed consent and willing to comply with the requirements and restrictions listed in the informed consent form and in this protocol

### Exclusion Criteria

Participants are excluded from the study if any of the following criteria apply:

1. Active or acute infection other than SARS-CoV-2, including secondary bacterial pneumonia
2. Presence of an acute or chronic condition that, as judged by the investigator, would jeopardize the safety of the participant
3. ALT or AST levels >5 times the upper level of normal
4. Severe or critical COVID-19 disease, i.e. requiring non-invasive or invasive mechanical ventilation, use of high-flow oxygen devices or ECMO
5. Underlying condition that may interfere with inhalation of the IMP
6. Allergy to niclosamide or history of significant adverse reaction to niclosamide or related compounds, or to any of the excipients used
7. Other investigational products within one month prior Day 1 and throughout the study
8. Enrolment in another study for the formulation in the previous 6 months
9. The presence of a condition the investigator believes would interfere with the ability to provide consent, or comply with study instructions, or that might confound the interpretation of the study results.

### Administration

All enrolled participants will receive niclosamide ethanolamine Nebulizer Solution 1% or placebo and niclosamide ethanolamine Nasal Spray Solution 1% or placebo in a twice-daily procedure for up to 10 days, while hospitalized. This treatment includes administration of the Nasal Spray Solution 1% or placebo as one spray shot of 130 μL of the solution in each nostril followed by the nebulization of 3 mL of the solution 1% or placebo. Treatment is to discontinue at hospital discharge.

### Efficacy Assessments

The modified ordinal scale for clinical improvement will be completed by the investigator. The scale is provided in Table 12 below. The SpO₂ value will be determined daily while hospitalized and by clinic staff on Day 28. Participants with an abnormal (<95%) SpO₂ on the day of discharge will be provided with a pulse oximeter with instructions. The participant will measure SpO₂ at home and report the value at the daily post-treatment visit. Sputum or nasopharyngeal swans for titer of SARS-CoV-2 will be collected daily while hospitalised and by clinic staff on Day 28. Samples will be collected for laboratory analysis of inflammatory biomarkers at the Screening, Day 10 (or day of discharge), and Day 28 visits.

TABLE 12
Modified ordinal scale for clinical improvement

| | COVID-19 | | |
|---|---|---|---|
| Participant status | severity | Descriptors integrating FDA guidance | Score |
| Asymptomatic | Clear | No symptoms | 0 |
| Ambulatory | Almost | Very mild symptoms of COVID-19 without limitations in | 1 |
| | clear | daily activities ^{a}. Oxygen saturation is normal (≥95% on | |
| | | room air) or equivalent to pre-COVID-19 oxygen | |
| | | requirement | |
| Ambulatory | Mild | Mild residual symptoms of COVID-19 with limitations in | 2 |
| | | daily activities and no need for specific medical | |
| | | oversight ^{b}. Oxygen saturation is normal at rest (≥95% | |
| | | on room air) or equivalent to pre-COVID-19 oxygen | |
| | | requirement | |
| Hospitalized | Mild | Mild active illness with COVID-19 without evidence of | 3 |
| | | active viral pneumonia or hypoxia but where | |
| | | hospitalization and/or close medical monitoring is | |
| | | needed ^{c}. Oxygen saturation is normal (≥95% on room | |
| | | air) or equivalent to pre-COVID-19 oxygen requirement | |
| Hospitalized | Moderate | Moderate illness with COVID-19 with clinical signs of | 4 |
| | | pneumonia but no signs of severe pneumonia. Oxygen | |
| | | saturation is >93% on room air at sea level | |
| Hospitalized | Severe | Severe illness with COVID-19 with clinical signs of | 5 |
| | | respiratory distress such as respiratory rate ≥30/ | |
| | | minute, heart rate ≥125/minute, SpO₂ ≤93% 6on | |
| | | room air at sea level or PaO₂/FiO₂ | |
| Hospitalized | Critical | Critical illness, defined by at least one of the following: | 6 |
| | | Respiratory failure ^{d}; Shock ^{e}; Multiorgan | |
| | | dysfunction/failure. | |
| Dead | Fatal | Death | 7 |

^{a} Persistent fatigue, dyspnea at effort, joint or chest pain, cough or persistent smell or taste dysfunction might be present.
^{b} Fatigue or dyspnea at rest preventing some daily activities are present. If participant is discharged from the hospital, but still needs close medical monitoring or oxygen therapy at home, they should be categorized in score 3.
^{c} Hospitalization required for medical supervision; can include participants at high risk of complication (eg, due to comorbidities) or those recovering from the disease but needing medical oversight before hospital discharge.
^{d} Requiring at least one of the following: endotracheal intubation and mechanical ventilation, oxygen delivered by high-flow nasal cannula (heated, humidified, oxygen delivered via reinforced nasal cannula at flow rates >20 L/min with fraction of delivered oxygen ≥0.5), non-invasive positive pressure ventilation, ECMO, or clinical diagnosis of respiratory failure (i.e., clinical need for one of the preceding therapies, but preceding therapies not able to be administered in setting of resource limitation).
^{e} Systolic blood pressure <90 mmHg or diastolic blood pressure <60 mmHg or requiring vasopressors.

### Pharmacokinetics & Biomarkers

Plasma, serum or whole blood samples will be collected for measurement of niclosamide concentrations.

Samples will be tested for C-reactive protein and procalcitonin to evaluate their association with observed clinical responses.

### Statistical Considerations

The null and alternative hypotheses for the primary efficacy outcome, time to clinical improvement, are the following:

H0: h1(t)=h2(t)
H1: h1(t)≠h2(t)

### Where:

h1(t) is the hazard function in the inhalation group
h2(t) is the hazard function in the placebo group

The remdesivir study of moderate and severe COVID-19 subjects found that the median time to recovery, defined as being a 1 (not hospitalized, no limitations of activity), 2 (not hospitalized, limitation of activities, home oxygen requirement or both) or 3 (hospitalized, not requiring supplemental oxygen and no longer requiring ongoing medical care) on the ordinal scale was 11 days versus 15 days for subjects receiving remdesivir and placebo, respectively, with a hazard ratio of 1.32. Since this study will enroll participants with mild and moderate COVID-19, the outcome is improvement rather than recovery, and some participants may be receiving concomitant remdesivir, it is expected that the time to clinical improvement will be shorter than in the remdesivir study. Assuming the median time to clinical improvement is 9.7 days in the inhalation 1% group and 13.55 days in the placebo group (hazard ratio of 1.40), 80% power, 2-sided alpha=0.05, and a 2:1 randomization ratio, a total of 328 events are required. Assuming an event rate of 90% and a 5% lost to follow-up rate, approximately 387 subjects need to be enrolled.

### Primary Endpoints

The primary efficacy outcome is time to clinical improvement (at least 2 grades in the modified ordinal scale) in the ITT analysis set. The description of the estimand includes four attributes: the population, the variable (or endpoint) to be obtained for each participant, the specification of how to account for intercurrent events (ICE), and the population-level summary for the variable. The estimand attributes for time to clinical improvement will be provided in detail in the SAP.

Kaplan-Meier (KM) analysis will be used to assess time to clinical improvement in the inhalation and placebo groups. Participants who do not improve (including death) or are lost to follow-up will be censored at the date of last assessment or date of death. Participants who receive rescue therapy with remdesivir will be censored at the start date of remdesivir treatment. The 25th percentile, median and 75th percentile for time to clinical improvement and 95% confidence intervals (CIs) will be determined in each treatment group. The hazard ratio for clinical improvement and 95% Cl will also be determined using a Cox regression model with covariates for treatment, COVID-19 severity (mild and moderate), country/geographic region, and age (<75 and ≥75 years). KM survival curves will be provided.

A log-rank test, stratified for the randomization factors of COVID-19 severity (mild and moderate), country/geographic region, and age (<75 and >75 years), will be performed to test for differences in survival curves between the two treatment groups. If the p-value is <0.05 (i.e., a 2-sided alpha level of 0.05), the null hypothesis will be rejected. Analyses of the primary efficacy outcome, time to clinical improvement, will also be conducted in the subgroups defined by COVID-19 severity (mild and moderate), country/geographic region, age (<75 and >75 years), and receipt of concomitant remdesivir (yes and no), as well as in the m-ITT and PP analyses sets. Other subgroup analyses will be defined in the SAP.

### Secondary Endpoint(s)

The following key secondary analyses will be completed in the ITT analysis set. To control for the inflation of the overall type I error rate, a hierarchical testing procedure will be used. If statistical significance is declared for the primary efficacy outcome, testing will be done for the key secondary efficacy outcomes in the order listed below. Testing will proceed to the next secondary outcome only if statistical significance (2-sided alpha=0.05) is declared for the preceding secondary outcome being tested.
A frequency distribution of the scores from the modified ordinal scale will be presented by treatment group at Day 14. Differences between treatment groups will be tested for statistical significance using a proportional-odds logistic regression with covariates for treatment and each of the randomization stratification factors. The odds ratio and 95% Cl for treatment will be presented.
The number and percentage of participants with respiratory failure defined as the need for high-flow oxygen, mechanical ventilation, ECMO or non-invasive ventilation will be presented by treatment group. The Cochran-Mantel-Haenzsel test, stratified for the randomization stratification factors, will be used to determine statistical significance between the treatment groups.
KM methods will be utilized to analyze time to no need for oxygen therapy or return to the oxygen level needed before COVID-19 disease with participants who do not return to pre-COVID-19 oxygen level, deaths or who are lost to follow-up censored at the date last assessed or last date known to be alive. Participants who receive rescue therapy with remdesivir will be censored at the start date of remdesivir treatment. Kaplan-Meier curves will be provided. The 25th, 75th percentiles and the median time to no need for oxygen therapy or return to oxygen level needed before COVID-19 as well as 95% CIs will be determined. The hazard ratio and 95% Cl will also be determined using a Cox regression model with covariates for treatment, COVID-19 severity (mild and moderate), country/geographic region, and age (<75 and >75 years). KM survival curves will be provided. Differences between survival curves will be tested for statistical significance using the log-rank test stratified by the randomization stratification factors.
Kaplan-Meier methods will be utilized to analyze survival time with participants who remain alive or are lost to follow-up censored at the last date known to be alive. Kaplan-Meier survival curves will be provided. The 25th, 75th percentiles and the median survival time as well as the probability of being alive at Day 28 will be determined by treatment group. Statistically significant differences between treatment groups in the probability of being alive at Day 28 will be determined using a Z-statistic and Greenwoods formula for the standard deviation.

Additional analyses of the key secondary endpoints will be conducted. These include analyses in the subpopulations defined by the randomization stratification factors and in the m-ITT and PP analysis sets. Summaries of additional secondary endpoints will also be conducted including:
Time to viral clearance defined as the time to the first of 2 consecutive negative tests for SARS-CoV-2 will be summarized using KM methods. Participants who do not have viral clearance (including deaths) or are lost to follow-up will be censored at the date of last viral test or date of death. Participants who receive rescue therapy with remdesivir will be censored at the start date of remdesivir treatment. The 25th percentile, median and 75th percentile for time to viral clearance and 95% CIs will be determined in each treatment group. The hazard ratio for viral clearance and 95% Cl will also be determined using a Cox regression model with covariates for treatment, COVID-19 severity (mild and moderate), country/geographic region, and age (<75 and >75 years). KM survival curves will be provided.
The probability of having viral clearance at each time point it is measured will be determined from the KM analysis.
Descriptive statistics of the mean viral burden (AUC of viral particle titers) during the 10-day treatment period will be provided.
Descriptive statistics of the peak viral load during the 10-day treatment period will be provided.

### Tertiary/Exploratory Endpoint(s)

Summaries of the additional efficacy outcomes by treatment group will be conducted in the ITT analysis set to support the findings of the primary and secondary efficacy outcomes as follows:
A frequency distribution of the scores from the modified ordinal scale at end of treatment/discharge and Day 28.
Number and percentage of participants cleared (modified ordinal scale=0) or almost cleared (modified ordinal scale=1) at Day 14, end of treatment/discharge and Day 28.
Time to clearance/almost clearance will be summarized using KM methods. Participants who do not have clearance/almost clearance (including death) or are lost to follow-up will be censored at the date of last assessment or date of death. Participants who receive rescue therapy with remdesivir will be censored at the start date of remdesivir treatment. The 25th percentile, median and 75th percentile for time to clearance/almost clearance and 95% CIs will be determined in each treatment group. The hazard ratio for clearance/almost clearance and 95% Cl will also be determined using a Cox regression model with covariates for treatment, COVID-19 severity (mild and moderate), geographic region (US and non-US), and age (<75 and >75 years). KM survival curves will be provided.
Number and percentage of participants with an improvement in the modified ordinal scale of at least 1 grade lower than baseline=0) at Day 14 and Day 28.
Number and percentage of participants worsened (defined as a modified ordinal scale score higher than baseline) at Day 14 and Day 28.
Number and percentage of participants hospitalized in an ICU at any time during the hospitalization.
Number and percentage of participants needing oxygen therapy at any time during the hospitalization.
Number and percentage of participants receiving mechanical ventilation at any time during the hospitalization.
Number and percentage of participants discharged from the hospital with at least a 2-grade improvement in the modified ordinal scale.
Time to hospital discharge with at least a 2-grade improvement in the modified ordinal scale. Participants who are not discharged (including death) or are lost to follow-up will be censored at the date of last assessment or date of death. Participants who receive rescue therapy with remdesivir will be censored at the start date of remdesivir treatment. The 25th percentile, median and 75th percentile for time to hospital discharge and 95% CIs will be determined in each treatment group. The hazard ratio for hospital discharge and 95% Cl will also be determined using a Cox regression model with covariates for treatment, COVID-19 severity (mild and moderate), geographic region (US and non-US), and age (<75 and >75 years). KM survival curves will be provided.
For participants who require oxygen therapy, descriptive statistics of the time to sustained return to basal oxygen requirement.
For participants who show a clinical improvement, the number and percentage with a relapse defined as rehospitalization for COVID-19 through Day 28.
Number and percentage of participants with a SpO2 value of ≤91%, 92-93%, 94-95%, and ≥95% at Day 14, end of treatment/discharge and Day 28.
Number and percentage of participants with a respiration rate (bpm) of ≤8, 9-11, 12-20, 21-24, and ≥25 at Day 14, end of treatment/discharge and Day 28.
Mean change from baseline to each time point measured in C-reactive protein and procalcitonin.
The number and percentage of participants with viral clearance at each time point measured using the subgenomic RNA assay.

### Example 10: Treatment of Asymptomatic or Mildly Symptomatic Patients with COVID-19

The following clinical trial may be performed using a formulation described herein, such as Formulation A described in Example 1 or the formulation described in Table 8, Example 6.

The overall objective of this phase 2 study is to demonstrate the benefit of treatment of asymptomatic or mildly symptomatic patients with recent proven SARS-CoV-2 infection, targeting patients who have an early stage of disease primarily involving the upper airways.

Primary objectives are to assess the:
Development and progression of COVID-19 symptoms
Safety of administration of the formulation
Viral shedding assessed by nasopharyngeal (NP) SARS-CoV-2 RT-PCR test at Day 5 and 10
Impact on long-term COVID symptoms
Determination of the trough niclosamide values following administration

### Primary Objective:

To assess the efficacy of the treatment to prevent disease progression
To assess the safety of the treatment

### Secondary Objective:

To assess the efficacy of treatment on symptoms of COVID-19
To assess the effect of treatment on SARS-CoV-2 viral load
To assess the effect of treatment on the spread of COVID-19 from the index case

### Primary Endpoints:

Change from baseline in symptoms through Day 10 defined aggregated Food and Drug Administration (FDA) COVID-19 questionnaire score from baseline through Day 10 comparing the formulation vs placebo.
Safety of the formulation nasal spray as assessed by adverse events, vital signs, haematology, and clinical chemistry.

### Secondary Endpoints:

Maximal intensity of symptoms in the modified FDA COVID-19 questionnaire
Number of days free of COVID-19 symptoms as defined by the FDA COVID-19 questionnaire
Patient-reported global impression items assessing a) return to usual health; b) return to usual activities; and c) overall COVID-19-related symptoms on Day 10: examples of patient-reported global impression item(s) as outlined in the FDA COVID-19 questionnaire
Proportion of patients remaining asymptomatic on Day 10
Proportion of patients requiring visits to urgent care (UC) or emergency department (ED) facilities, or hospitalization due to signs or symptoms of COVID-19
Proportion of patients admitted to intensive care units (ICU)
Change from baseline in SARS-CoV-2 viral load at Day 10 as assessed by quantitative reverse transcription polymerase chain reaction (qRT-PCR).
Change from baseline in SARS-CoV-2 viral load at Day 5 as assessed by qRT-PCR.
Presence of long-term COVID-19 symptoms as defined by the FDA COVID-19 questionnaire.

### Exploratory Endpoints:

Time to reaching a score 2 in the WHO 11-point ordinal scale
Proportion of patients reaching a score 2 in the 11-point WHO ordinal scale
Percentage of patients spreading SARS-CoV-2 to household contacts up to Day 30
Viral shedding assessed by results from a positive or negative NP SARS-CoV-2 reverse transcription polymerase chain reaction (RT-PCR) test
Persistence/appearance of symptoms (long COVID-19) post-day 10
Patient reported global impression items assessing a) return to usual health; b) return to usual activities; and c) overall COVID-19-related symptoms on Day 10 and Day 30.

### Overall Study Design

Given that a nasal compound is unlikely to be able to eradicate virus that have reached the lower parts of the respiratory tree or otherwise has spread systemically, this study will focus on SARS-CoV-2-positive patients with asymptomatic or mildly symptomatic disease primarily involving the upper airways. The patient population is defined to comprise of patients with either no symptoms, or symptoms such as nasal congestion, runny nose, conjunctivitis, sore throat, loss of taste, loss of smell, headache. Individuals with symptoms suggesting engagement of the lower respiratory tract or a systemic engagement such as cough, feeling feverish, chills, shivering, feeling hot, low energy, tiredness, body aches and pains, fatigue, shortness of breath, loss of appetite, nausea, vomiting, or diarrhoea will be excluded. A maximum of 50% of the participants will have mildly symptomatic COVID-19 disease, the remaining participants will have no symptoms.

The development of a composite set of symptoms will be collected on the FDA COVID-19 questionnaire to be filled out by the patient at the same time (±1 hour) every day. The time should be convenient for the patients but cannot be completed within 2 hours of waking up. The questionnaire should be completed daily before investigational medicinal product (IMP) intake. The responses will be collected directly by telephone by a HCP every other day during the treatment period. All patients will be followed until Day 30. At Day 30, the patient will be asked by the HCP to evaluate their symptoms during the past week and record the highest severity during that week to evaluate the presence of long-term COVID-19.

On Day 10 and Day 30 evaluation of patient-reported global impression items assessing a) return to usual health; b) return to usual activities; and c) overall COVID-19-related symptoms (examples of patient-reported global impression item(s) as outlined in the FDA COVID-19 questionnaire) with the following questions:
Have you returned to your usual health (before your COVID-19 illness)? Yes or No
Have you returned to your usual activities (before your COVID-19 illness)? Yes or No
In the past 24 hours, what was the severity of your overall COVID-19-related symptoms at their worst? None, Mild, Moderate, or Severe.

Assessments of clinical status using the WHO ordinal 11-point scale will occur at screening, Day 1, and every other day until Day 10 and Day 30 by a HCP.

The ability of the treatment to prevent transmission of SARS-CoV-2 to household contacts will be assessed by using a set of questions asked by a HCP. The responses will be collected by a phone call from a HCP every other day during the treatment and follow-up periods (Days 1, 3, 5, 7, 9, 10, and 30). At the follow-up phone visit on Day 30 AEs and information on transmission to household contact(s) will be collected. In participants showing a worsening of the signs and symptoms of COVID-19, treatment should continue up to day 10, unless the investigator decides to discontinue treatment for safety reasons. The investigator may also decide that continued treatment is not feasible due to the respiratory status of the participant.

Assessment of presence and duration of unpleasantness following administration of the treatment will be assessed daily during Day 1-10 with the following question: Did you experience any unpleasantness after taking the nasal spray? If yes, what was the symptom and how long did it last? Responses could be a reason for AE reporting.

The study duration for an individual patient will be as follows:
Screening period: up to 2 days
Treatment period: 10 consecutive days (ie, Day 1 to Day 10)
Follow-up period: 30 days after the patient's first treatment with the study drug (ie, Day 30)

### Inclusion Criteria

1. The patient is male or female aged ≥45 years.
2. The patient is able to understand and provide signed informed consent.
3. The patient is tested to confirm infection with SARS-CoV-2 by lateral flow antigen test or RT-PCR on a sample taken within 3 days before randomization.
4. The patient is either without symptoms or has one or more of the following symptoms: stuffy or runny nose, conjunctivitis, sore throat, loss of taste, loss of smell, or headache (to be entered in the FDA COVID-19 questionnaire). Runny nose and conjunctivitis are also acceptable.
5. Men whose sexual partners are women of childbearing potential (WOCBP) must agree to comply with one of the following contraception requirements from the time of first dose of screening until at least 30 days after the last dose of study medication:
a. Vasectomy with documentation of azoospermia.
b. Sexual abstinence (defined as refraining from heterosexual intercourse from the time of screening until at least 30 days after the last dose of study medication)
c. Male condom plus partner use of one of the contraceptive options below: contraceptive subdermal implant; intrauterine device or intrauterine system; oral contraceptive, either combined or progestogen alone; injectable progestogen; contraceptive vaginal ring; percutaneous contraceptive patches.

The above is an all-inclusive list of those methods that meet the following definition of highly effective: having a failure rate of less than 1% per year when used consistently and correctly and, when applicable, in accordance with the product label. For non-product methods (e.g., male sterility), the investigator will determine what is consistent and correct use. The investigator is responsible for ensuring that patients understand how to properly use these methods of contraception.

6. WOCBP must agree to comply with one of the following contraception requirements from the time of screening until at least 30 days after the last dose of study medication:
a. Sexual abstinence (defined as refraining from heterosexual intercourse from the time of screening until at least 30 days after the last dose of study medication)
b. Use of one of the contraceptive options below plus use of a condom by male partner: contraceptive subdermal implant; intrauterine device or intrauterine system; oral contraceptive, either combined or progestogen alone; injectable progestogen; contraceptive vaginal ring; percutaneous contraceptive patches.
c. Vasectomy of male partner with documentation of azoospermia.

The above is an all-inclusive list of those methods that meet the following definition of highly effective: having a failure rate of less than 1% per year when used consistently and correctly and, when applicable, in accordance with the product label. The investigator is responsible for ensuring that patients understand how to properly use these methods of contraception.

7. Women of non-reproductive potential are defined as: a) Premenopausal females with one of the following: documented tubal ligation; documented hysteroscopic tubal occlusion procedure with follow-up confirmation of bilateral tubal occlusion; hysterectomy; documented bilateral oophorectomy. b) Postmenopausal defined as 12 months of spontaneous amenorrhea (in questionable cases a blood sample will be required with simultaneous follicle stimulating hormone and estradiol levels tested locally and consistent with menopause [refer to local laboratory reference ranges for confirmatory levels]). Women on hormone replacement therapy (HRT) and whose menopausal status is in doubt will be required to use one of the highly effective contraception methods listed above if they wish to continue their HRT during the study.

### Exclusion Criteria

1. The patient has been enrolled in a study with niclosamide in the previous 6 months.
2. The patient is allergic to niclosamide or has a history of a significant adverse reaction to niclosamide or related compound, or to any of the excipients used.
3. The patient has an underlying condition that may interfere with intranasal administration of the IMP, for example chronic ulcer(s) in the nose.
4. The patient has an acute or chronic condition that, as judged by the investigator, would jeopardize the safety of the participant.
5. The patient has a condition the investigator believes would interfere with the ability to provide consent, or comply with study instruction, or that might confound the interpretation of the study results.
6. Patients with symptoms suggesting engagement of the lower respiratory tract or a systemic engagement such as cough, feeling feverish, chills, shivering, feeling hot, low energy, tiredness, body aches and pains, fatigue, shortness of breath, loss of appetite, nausea, vomiting, or diarrhea (to be entered in the FDA COVID-19 questionnaire), or other symptoms not mentioned in Inclusion Criteria 5.
7. The patient has an active or acute infection other than SARS-CoV-2.
8. The patient has used other investigational products the month prior to Day 1.
9. Antiviral medications and approved or experimental medications targeting COVID-19
10. Another member of the same household recruited to this study.

### Treatment

The treatment is a nasal spray with a novel triple mechanism of action including antiviral, antibacterial, and anti-inflammatory properties. The treatment and matching placebo will be supplied in 20 mL amber glass vials with mounted nasal spray pumps, containing 8.5 mL of the respective solution, delivering 140 μL per spray shot. Both are isotonic and euhydric aqueous solutions with yellow/red colour. The IMP and matching placebo will be provided by a manufacturer independent to the trial and will be stored between 2-8° C., and then between 15-25° C. following dispensation to patients.

All randomized patients will receive 140 μL of formulation 1% or placebo in each nostril (2.4 mg niclosamide) administered BID from Day 1 (2 doses taken a minimum of 6 hours apart) to the last dose on Day 10, with only one dose taken the morning of Day 10, according to their assigned treatment and according to the randomization scheme. The study is double-blinded.

### Efficacy Assessments

The patient will be assessed using the FDA COVID-19 questionnaire as shown in Table 13 answering questions 1-16 on Days 1-9; questions 1-19 on Day 10; and questions 1-18 on Day 30 (assessing the past 7 days). Question 20-22 will be answered on Day 10 (assessing the past 10 days) and on Day 30 (assessing the past 20 days). At Day 10 and Day 30 patient-reported global impression items (questions 17-19) assessing a) return to usual health; b) return to usual activities; and c) overall COVID-19-related symptoms will also be collected as part of above COVID-19 Symptoms questionnaire.

Health care utilization will be assessed with the WHO 11-point Ordinal Scale (Table 14). The ability of the treatment to prevent transmission of SARS-CoV-2 to household contacts will be assessed using a set of questions (Table 15) asked by a Health Care Professional (HCP). The responses will be collected by a phone call from a HCP every other day during the treatment and follow-up periods. At the follow-up phone visit on Day 30 information on transmission to household contact(s) will be collected.

The presence of symptoms remaining on Day 30 will be assessed using the questions 1-18 from Table 13 in the FDA COVID-19 questionnaire to be asked by a HCP at a remote visit/telephone call. The patient will be asked on Day 30 to evaluate their symptoms during the past week and record the highest severity during that week on the FDA COVID-19 questionnaire.

The presence and duration of unpleasantness following administration of the formulation will be assessed daily with the following question at Day 1-10:
Did you experience any unpleasantness after taking the nasal spray? If yes, what was the symptom and how long did it last?

TABLE 13
FDA COVID-19 Questionnaire

| | Response Options |
|---|---|
| Symptom | and Scoring |
| 1. Stuffy or runny nose | None = 0 |
| 2. Sore throat | Mild = 1 |
| 3. Shortness of breath (difficulty | Moderate = 2 |
| breathing) | Severe = 3 |
| 4. Cough | |
| 5. Low energy or tiredness | |
| 6. Muscle or body aches | |
| 7. Headache | |
| 8. Chills or shivering | |
| 9. Feeling hot or feverish | |
| 10. Nausea (feeling like you wanted to | |
| throw up) | |
| 11. Overall in the past 24 hours, what | |
| was the severity of your COVID-19-related | |
| symptoms? | |
| 12. How many times did you vomit (throw | I did not vomit at |
| up) in the last 24 hours? | all = 0 |
| | 1-2 times = 1 |
| | 3-4 times = 2 |
| | 5 or more times = 3 |
| 13. How many times did you have | I did not have diarrhea |
| diarrhea (loose or watery stools) in the last | at all = 0 |
| 24 hours? | 1-2 times = 1 |
| | 3-4 times = 2 |
| | 5 or more times = 3 |
| 14. Rate your sense of smell in the last | My sense of smell is THE |
| 24 hours. | SAME AS usual = 0 |
| | My sense of smell is LESS |
| | THAN usual = 1 |
| | I have NO sense of |
| | smell = 2 |
| 15. Rate your sense of taste in the last 24 | My sense of taste is THE |
| hours. | SAME AS usual = 0 |
| | My sense of taste is LESS |
| | THAN usual = 1 |
| | I have NO sense of |
| | taste = 2 |
| 16. Did you experience any | Yes or No |
| unpleasantness after taking the nasal | |
| spray?* | |
| 17. Have you returned to your usual | Yes or No |
| health (before your COVID-19 illness?) | |
| 18. Have you returned to your usual | |
| activities (before your COVID-19 illness?) | |
| 19. Did you think you had any benefit | |
| from the treatment received? | |
| 20. How many times have you been in | Number of occurrences for |
| phone contact with your physician or the | the following (if any) |
| hospital? | |
| 21. How many times have you visited | Number of occurrences for |
| your physician/hospital (ambulatory or | the following (if any) |
| emergency department)? | |
| 22. How many times have you been | Number of occurrences for |
| admitted to the hospital? | the following (if any) |

TABLE 14
WHO 11-Point Ordinal Scale

| Patient State | Descriptor | Score |
|---|---|---|
| Uninfected | Uninfected; no viral RNA detected | 0 |
| Ambulatory mild disease | Asymptomatic; viral RNA detected | 1 |
| | Symptomatic; independent | 2 |
| | Symptomatic; assistance needed | 3 |
| Hospitalized: moderate disease | Hospitalized; no oxygen therapy | 4 |
| | Hospitalized; oxygen by masks or nasal | 5 |
| | prongs | |
| Hospitalized: severe diseases | Hospitalized oxygen by NIV or high flow | 6 |
| | Intubation and mechanical ventilation, | 7 |
| | pO₂/FiO₂ ≥150 or SpO₂/FiO₂ ≥200 | |
| | Mechanical ventilation pO₂/FiO₂ <150 | 8 |
| | (SpO₂/FiO₂ <200) or vasopressors | |
| | Mechanical ventilation pO₂/FiO₂ <150 and | 9 |
| | vasopressors, dialysis, or ECMO | |
| Dead | Dead | 10 |

TABLE 15
Prevention of Transmission to Household Contacts

| | |
|---|---|
| Question | Response Option |

Questions to be asked at Day 1 visit (baseline)

| | |
|---|---|
| 1. Number of people in your household, | If the answer is ‘1’, the questions are NA for the |
| including yourself? | remaining of the trial. |
| 2. Are you believed to be the initial | Yes or No |
| person in your household to be infected? | |
| 3. How many of the household members | Number: |
| (excluding yourself) have previously been | (if ≥1 please ask the below question) |
| confirmed positive with COVID-19? | |
| 3a. Please state which type of COVID-19 test | PCR; quicktest (lateral flow antigen test); |
| was used | homekit |
| | Start by asking if the household member(s) had |
| | a positive PCR test. If yes, refrain from asking |
| | about the other tests. If no PCR, ask if the |
| | Quicktest was positive, and lastly Homekit. |
| 4. How many of the household members | Number: |
| (excluding yourself) are showing symptoms of | (if ≥1 please ask the below question) |
| COVID-19? | |
| 4a. When did the first symptom(s) appear for | List date for each household member showing |
| your household members? | symptoms of COVID-19 (DDMMMYYYY). |
| | Date: |

Questions to be asked at Day 3, 5, 7, 9, and 10 treatment
visits and Day 30 follow-up visit, as applicable

| | |
|---|---|
| 1. How many of the household members | Number: |
| have since your last contact with the site been | (if ≥1 please ask the below question) |
| confirmed position with COVID-19? | |
| 1a. Please state which type of COVID-19 test | PCR; quicktest (lateral flow antigen test); |
| was used | homekit |
| | Start by asking if the household member(s) had |
| | a positive PCR test. If yes, refrain from asking |
| | about the other tests. If no PCR, ask if the |
| | Quicktest was positive, and lastly Homekit. |
| 2. How many of the household member | Number: |
| have since your last contract with the site been | (if ≥1 please ask the below question) |
| showing any of the symptoms of COVID-19? | |
| 2a. When did the first symptoms(s) appear for | List date for each household member showing |
| your household members? | symptoms of COVID-19 (DDMMMYYYY). |
| | Date: |

### Example 11: Niclosamide Ethanolamine Salt is Effective Against Several SARS-CoV-2 Variants, Including the Variants of Concern of the Lineage B.1.1.7 (UK) and B.1.351 (South Africa)

### Methods

The effect of niclosamide ethanolamine salt (NEN) on the replication of several variants of SARS-CoV-2 was determined as previously described with the below outlined deviations (Touret et al., 2020, Preclinical evaluation of Imatinib does not support its use as an antiviral drug against SARS-CoV-2. bioRxiv).

Caco-2 cells were cultivated similar to VeroE6 cells as described in Touret et al., 2020. VeroE6 TMPRSS2 cells (ID 100978) were obtained from CFAR and were grown in the same medium with the addition of G-418 (Life Technologies). SARS-CoV-2 strain BavPat1 was obtained from Pr. C. Drosten through EVA GLOBAL (https://www.european-virus-archive.com/). SARS-CoV-2 201/501YV.1 was isolated from a 18 years-old patient. The full genome sequence has been deposited on GISAID: EPI_ISL_918165. The strain is available through EVA GLOBAL: UVE/SARS-CoV-2/2021/FR/7b (lineage B 1. 1.7, ex UK) at https://www.european-virus-archive.com/virus/sars-cov-2-uvesars-cov-22021fr7b-lineage-b-1-1-7-ex-uk. SARS-CoV-2 Wuhan D614 strain generated by ISA method. It contains the original D614 residue on the Spike protein. The strain is available through EVA GLOBAL UVE/SARS-CoV2/2020/FR/ISA_D614 at https://www.european-virus-archive.com/virus/sars-cov-2-virus-strain-uvesars-cov22020frisad614. SARS CoV-2 SA (lineage B 1.351) was isolated in France in 2021, The strain is available through EVA GLOBAL: UVE/SARS-CoV-2/2021/FR/1299-ex SA (lineage B 1.351) at https://www.european-virus-archive.com/virus/sars-cov-2-uvesars-cov-22021fri299-ex-sa-lineage-b-1351. A half-log dilution scheme using concentrations from 10 μM to 0.078 μM was used for niclosamide ethanolamine salt (solubilised in DMSO) in VeroE6 cells and using concentrations from 5 μM to 0.039 μM in VeroE6 TMPRSS2 cells. The EC₅₀ and CC₅₀ determination was carried out as described in Touret et al., 2020.

### Results

NEN was found to inhibit the replication of SARS-CoV-2 (D614G strain) in VeroE6 cells with an EC₅₀ of 0.1 μM and a CC₅₀ of >10 μM yielding a Selectivity Index of 100 (FIG. 5A). The potent antiviral efficacy of NEN was confirmed in Caco-2 cells showing an EC₅₀ of 0.08 μM and a CC₅₀>10 μM (FIG. 5B).

To ensure appropriate replication of SARS-CoV-2's variants, the cell line VeroE6 TMPRSS2 was employed. Treatment with NEN blocked the replication of all four SARS-CoV-2's variants with a similar potency (FIG. 6). More precisely, the EC₅₀ against the D614G, D614, B.1.1.7 and B 1.351 strain was 0.06 μM, 0.13 μM, 0.08 μM and 0.07 μM, respectively.

### Example 12: Niclosamide Blocks Replication of SARS-CoV-2 in a Trans-Well Model of Infection Using Human Bronchial Epithelial Cells

Methods

The impact of niclosamide ethanolamine salt (NEN) on replication of SARS-CoV-2 was evaluated in a transwell model of infection using human bronchial epithelial cells as previously described (Touret et al., 2020).

Briefly, human bronchial epithelial cells were apically infected with the European D614G strain of SARS-CoV-2 (BavPat1/2020; obtained from EVA GLOBAL) at a MOI of 0.1 and cultivated in basolateral media that contained different concentrations of NEN solubilised in DMSO (in duplicates) or no drug (virus control) for up to 4 days. Media was renewed daily containing fresh NEN. Samples were collected at the apical side and used to perform a TCID₅₀ assay. On day 4, cells were lysed to quantify intracellular viral RNA using qRT-PCR. The viral inhibition was calculated by normalizing the response, having the bottom value as 100% and top value as 0%. The EC₅₀ was determined using logarithmic interpolation (Y=100/(1+10\hat{ }((Log EC50−X)*HillSlope) in GraphPad Prims 7. Statistical tests were performed using Ordinary one-way ANOVA with Dunnett's correction for multiple comparisons.

### Results

NEN was found to exhibit a strong anti-SARS-CoV-2 effect when measured both by infectious titer and intracellular RNA levels in human bronchial epithelial cells.

Treatment with 1.25-10 μM of NEN resulted in a significant reduction of infectious titer of SARS-CoV-2 to levels below the limit of detection on Day 4 yielding an EC50 of 0.96 μM (FIG. 7A). Furthermore, treatment with 1 μM NEN significantly reduced the intracellular viral RNA level by 3-fold on day 4, compared to the non-treated control (FIG. 7B).

### Example 13: Intranasal Application of a Formulation of the Invention Leads to an Improved Clinical Score in SARS-CoV-2-Infected hACE2 Mouse Model

Methods

A liquid niclosamide ethanolamine composition according to the invention (as shown in Table 8 of Example 6) was given intranasally (IN) once prior to intranasal (IN) SARS-CoV-2 virus challenge and once daily after inoculation during the course of the study. The study used the hACE2-transgenic SARS-CoV-2 mouse model of infection established at the La Jolla Institute for Immunology in Sujan Shresta's laboratory (Oladunni et al., 2020, Nature communications, 11(1), pp. 1-17) where inoculation with SARS-CoV-2 results in severe SARS-CoV-2 related disease and early death by day 5-8. Virus infection was done with a 1.0×10⁵ PFU dose of SARS-CoV-2 WT (BEl Resources, diluted in PBS+10% FCS) intranasally in a final volume of 30 μL following isoflurane sedation. The composition and saline were administered in a volume of 30 μL. After viral infection, mice were monitored daily for morbidity (body weight), clinical scoring and mortality (survival). Mice showing >20% loss of their initial body weight and/or clinical score ≥5 were defined as reaching experimental end-point and humanely terminated.

### Results

Intranasal treatment with the composition of the invention resulted in a significant lower clinical score on Day 6 post-infection compared to saline-treated SARS-COV-2 infected K18hACE2 transgenic mice (FIG. 8). Table 16 below describes symptoms relating to the clinical score.

TABLE 16
Clinical score and symptoms

| Score | Description |
|---|---|
| 3 | Ruffled - Ruffled coat throughout body; A “wet” |
| | appearance; Active, scurrying, burrowing; Alert |
| 4 | Sick - Very ruffled coat; slightly closed inset |
| | eyes; walking but no scurrying; mildly lethargic |
| 5 | Very Sick - Very ruffled coat; closed inset |
| | eyes; slow to no movement; extremely |
| | lethargic (need to be sacrificed due to animal |
| | welfare) |

1. An inhalable pharmaceutical composition comprising niclosamide, or a pharmaceutically acceptable salt thereof, for use in the prevention or treatment of a viral infection in a subject, wherein the niclosamide, or a pharmaceutically acceptable salt thereof, is administered to the subject by inhalation.

2. The composition for the use of claim 1, wherein the pharmaceutical composition is in the form of a powder, a suspension or a solution comprising niclosamide, or a pharmaceutically acceptable salt thereof.

3. The composition for the use of claim 1, wherein the pharmaceutical composition is in the form of a solution or suspension comprising niclosamide, or a pharmaceutically acceptable salt thereof, preferably a liquid solution or liquid suspension.

4. The composition for the use of claim 3, wherein the solution or suspension comprises niclosamide, or a pharmaceutically acceptable salt thereof, and polyethylene glycol (PEG).

5. The composition for the use of claim 4, wherein the PEG has an average molecular weight of less than about 600.

6. The composition for the use of claim 4 or claim 5, wherein the PEG has an average molecular weight of from about 150 to about 600, for example an average molecular weight of about 200 or about 400.

7. The composition for the use of any one of claims 4 to 6, wherein the PEG is present in an amount of at least 25% by weight of the composition, for example, wherein the PEG is present in an amount of at least 30%, at least 35%, at least 40%, at least 45%, at least 50%, at least 55%, at least 60%, at least 65%, at least 70%, at least 75%, at least 80%, at least 85%, at least 90%, at least 95% or at least 98% by weight of the composition.

8. The composition for the use of any one of claims 4 to 6, wherein the PEG is present in an amount of from about 40% to about 98% by weight of the composition, for example from about 70% to about 96% by weight of the composition.

9. The composition for the use of any one of claims 3 to 8, wherein the solution or suspension further comprises a solvent (e.g. a glycol such as propylene glycol).

10. The composition of any one of claims 3 to 9, wherein the composition is in the form of a suspension comprising niclosamide, or a pharmaceutically acceptable salt thereof.

11. The composition for the use of any one of claims 3 to 9, wherein the composition is in the form of a solution comprising niclosamide, or a pharmaceutically acceptable salt thereof.

12. The composition for the use of any one of claims 3 to 11, wherein the composition is administered to the subject in the form of an aerosol of the solution or suspension comprising niclosamide, or a pharmaceutically acceptable salt thereof.

13. The composition for the use of any one of claims 1 to 12, wherein the composition comprises niclosamide in the free-acid form.

14. The composition for the use of any one of claims 1 to 12, wherein the composition comprises a pharmaceutically acceptable salt of niclosamide.

15. The composition for the use of any one of claims 1 to 12, wherein the composition comprises niclosamide ethanolamine.

16. The composition for the use of any one of claims 1 to 15, wherein the niclosamide or a pharmaceutically acceptable salt thereof is present in the composition in an amount of from about 0.1% to about 10% by weight of the composition, for example about 5% by weight of the composition.

17. The composition for the use of claim 1, wherein the composition is a solution comprising from about 1% to about 10% by weight niclosamide ethanolamine and PEG, wherein the PEG has an average molecular weight of less than 600.

18. The composition for the use of claim 1, wherein the composition is a solution comprising from about 4.5% to about 6.5% by weight niclosamide ethanolamine and about 93.5% to about 95.5% by weight PEG 400.

19. The composition for the use of claim 17 or claim 18, wherein the solution is administered to the subject in the form of an aerosol of the solution.

20. The composition for the use of any one of claims 1 to 19, wherein the composition further comprises a taste-masking agent.

21. The composition for the use of claim 20, wherein the taste-masking agent is selected from a sugar (e.g. sucrose, dextrose, or lactose), an amino acid or amino acid derivative (e.g. arginine, lysine, or monosodium glutamate), an oil (e.g. a natural oil, or plant extract), a sweetener (e.g. sucrose, dextrose, aspartame, acesulfame-K, sucralose or saccharin), an organic acid (e.g. citric acid or aspartic acid), and maltodextrin.

22. The composition for the use of any one of claims 1 to 21, wherein the composition is a non-aqueous composition, for example a composition containing less than 2% by weight water, preferably less than 0.1% by weight water, more preferably wherein the composition is anhydrous.

23. A method for preventing or treating a viral infection is a subject, the method comprising administering to the subject by inhalation an effective amount of an inhalable pharmaceutical composition comprising niclosamide, or a pharmaceutically acceptable salt thereof.

24. The method of any claim 23, wherein the composition is as defined in any one of claims 2 to 22.

25. The composition for use, or method of any one of claims 1 to 24, wherein the niclosamide, or a pharmaceutically acceptable salt thereof, is administered to the subject in a daily dose of from about 1 mg to about 3000 mg based on the weight of niclosamide (for example, from about 400 mg to about 2000 mg based on the weight of the niclosamide).

26. The composition for use, or method of any one of claims 1 to 25, wherein the niclosamide, or a pharmaceutically acceptable salt thereof, is administered to the subject in a unit dosage of from about 10 mg to about 1000 mg based on the weight of niclosamide (for example from about 100 mg to about 600 mg, preferably about 150 mg to about 500 mg, based on the weight of niclosamide).

27. The composition for use, or method of any one of claims 1 to 26, wherein the composition is administered by inhalation intraorally and/or intranasally, preferably wherein the niclosamide or solution is administered by inhalation intraorally.

28. The composition for use, or method of any one of claims 1 to 26, wherein the composition is administered intranasally.

29. The composition for use, or method of any one of claims 1 to 28, wherein the composition is administered to the subject one to five times per day, for example from 1 to 4 times per day.

30. The composition for use, or method of any one of claims 1 to 29, wherein the subject is treated with an antitussive agent prior to or concurrently with the inhaled niclosamide, or a pharmaceutically acceptable salt thereof.

31. The composition for use, or method of claim 30, wherein the antitussive agent is selected from codeine, dextromethorphan, hydrocodone, methadone, butorphanol, benzonatate, ethylmorphine, oxeladin, pipazethate, pholcodine, noscapine, butamirate and a local anaesthetic.

32. The composition for use, or method of claim 30, wherein the antitussive agent is a local anaesthetic, preferably lidocaine.

33. The composition for use, or method of any one of claims 30 to 32, wherein antitussive agent is administered to the subject prior to the inhaled niclosamide.

34. The composition for use, or method of any one of claims 1 to 33, wherein the viral infection is a pulmonary viral infection.

35. The composition for use, or method of any one of claims 1 to 34, wherein the viral infection is caused by or associated with a virus selected from respiratory syncytial virus, influenza virus, parainfluenza virus, human metapneumovirus, severe acute respiratory syndrome coronavirus (SARS-CoV), severe acute respiratory syndrome coronavirus (SARS-CoV-2), Middle East respiratory syndrome coronavirus (MERS-CoV), a human rhinovirus (HRVs) and human adenovirus (HAdV).

36. The composition for use, or method of any one of claims 1 to 34, wherein the viral infection is caused by or associated with a Pneumoviridae virus, for example a Human respiratory syncytial virus (HRSV) (e.g. HRSV-A2, HRSV-B1 or HRSV-S2).

37. The composition for use, or method of any one of claims 1 to 34, wherein the viral infection is caused by or associated with a Coronaviridae virus.

38. The composition for use, or method of claim 35, wherein the virus is selected from Alphacoronavirus, Betacoronavirus, Gammacoronavirus and Deltacoronavirus.

39. The composition for use, or method of claim 36, wherein the virus is a Betacoronavirus.

40. The composition for use, or method of claim 37, wherein the virus is selected from severe acute respiratory syndrome coronavirus (SARS-CoV), severe acute respiratory syndrome coronavirus 2 (SARS-CoV-2), Middle East respiratory syndrome coronavirus (MERS-CoV), HCoV-229E, HCoV-NL63, HCoV-OC43 and HKU1.

41. The composition for use, or method of any one of claims 1 to 33, wherein the viral infection is caused by or associated with SARS-CoV-2.

42. The composition for use, or method of any one of claims 1 to 33, wherein the viral infection is COVID-19, optionally wherein the viral infection is moderate or mild COVID-19.

43. The composition for use, or method of any one of claims 1 to 33, wherein the viral infection is caused by or associated with influenza virus.

44. An aerosol of a solution comprising niclosamide, or a pharmaceutically acceptable salt thereof.

45. The aerosol of claim 44, wherein the solution comprises niclosamide, or a pharmaceutically acceptable salt thereof, and polyethylene glycol (PEG).

46. The aerosol according to claim 44 or claim 45, wherein the PEG has an average molecular weight of less than about 600.

47. The aerosol of any one of claim 45 or claim 46, wherein the PEG has an average molecular weight of from about 150 to about 600, for example an average molecular weight of about 200 or about 400.

48. The aerosol of any one of claims 45 to 47, wherein the PEG is present in an amount of at least 25% by weight of the solution, for example, wherein the PEG is present in an amount of at least 30%, at least 35%, at least 40%, at least 45%, at least 50%, at least 55%, at least 60%, at least 65%, at least 70%, at least 75%, at least 80%, at least 85%, at least 90%, at least 95% or at least 98% by weight of the solution.

49. The aerosol of any one of claims 45 to 48, wherein the PEG is present in an amount of from about 40% to about 98% by weight of the solution.

50. The aerosol of any one of claims 44 to 49, wherein the solution comprises a solution of niclosamide in the free-acid form.

51. The aerosol of any one of claims 44 to 49, wherein the solution comprises a solution of a pharmaceutically acceptable salt of niclosamide.

52. The aerosol of any one of claims 44 to 49, wherein the solution comprises a solution of niclosamide ethanolamine.

53. The aerosol of any one of claims 44 to 52, wherein the niclosamide or a pharmaceutically acceptable salt thereof is present in the solution in an amount of from 0.1% to 10% by weight of the solution, for example about 5% by weight of the solution.

54. The aerosol of claim 44, wherein the composition is a solution comprising from about 4.5% to about 6.5% by weight niclosamide ethanolamine and about 93.5% to about 95.5% by weight PEG 400.

55. The aerosol of any one of claims 44 to 54, wherein the solution further comprises a taste-masking agent.

56. The aerosol of claim 55, wherein the taste-masking agent is selected from a sugar (e.g. sucrose, dextrose, or lactose), a amino acid or amino acid derivative (e.g. arginine, lysine, or monosodium glutamate), an oil (e.g. a natural oil, or plant extract), a sweetener (e.g. aspartame, acesulfame-K, sucralose or saccharin), an organic acid (e.g. citric acid or aspartic acid), and maltodextrin.

57. The aerosol of any one of claims 44 to 56, wherein the solution further comprises a solvent (e.g. e.g. a glycol such as propylene glycol).

58. The aerosol of any one of claims 44 to 56, wherein the solution is a non-aqueous solution, for example a solution containing less than 2% by weight water, preferably less than 0.1% by weight water, more preferably wherein the solution is anhydrous.

59. The aerosol of any one of claims 44 to 58, wherein the mass median diameter (MMD) of the aerosol is less than about 5 μm.

60. The aerosol of any one of claims 44 to 58, wherein the mass median diameter (MMD) of the aerosol is less than about 2 μm.

61. The aerosol of any one of claims 44 to 58, wherein the mass median diameter (MMD) of the aerosol is from about 0.5 μm to about 5.5 μm, preferably from about 1 μm to about 5 μm.

62. The aerosol of any one of claims 44 to 58, wherein the mass median diameter (MMD) of the aerosol is less than about 500 μm, preferably less than about 200 μm.

63. The aerosol of any one of claims 44 to 58, wherein the mass median diameter (MMD) of the aerosol is from about 5 to about 150 μm, preferably from about 20 to about 100 μm.

64. An aerosol according to any one of claims 44 to 63, for use in the prevention or treatment of a viral infection in a subject, wherein the aerosol is administered to the subject by inhalation.

65. An aerosol according to any one of claims 44 to 63, for use in the prevention or treatment of a viral infection in a subject, wherein the aerosol is administered to the subject intranasally.

66. An inhalable unit dosage comprising a solution of niclosamide, or a pharmaceutically acceptable salt thereof, and PEG, wherein niclosamide is present in an amount of from 1 mg to 600 mg based on the weight of niclosamide, for example from about 150 mg to about 500 mg, based on the weight of niclosamide.

67. An inhalable unit dosage comprising a solution of niclosamide ethanolamine and PEG, wherein niclosamide ethanolamine is present in an amount of from 2 mg to 600 mg, for example from about 150 mg to about 500 mg.

68. The inhalable unit dosage according to claim 66 or 67, wherein the unit dosage form is present in a container, for example a vial, blister pack, bottle, syringe or drug reservoir within an inhaler device (e.g. a nebulizer).

69. The inhalable unit dosage according to any one of claims 66 to 68, wherein the solution is a non-aqueous solution, for example a solution containing less than 2% by weight water, preferably less than 0.1% by weight water, more preferably wherein the solution is anhydrous.

70. A system comprising a container comprising a solution of niclosamide, or a pharmaceutically acceptable salt thereof, and polyethylene glycol (PEG); and an inhaler device and/or an intranasal delivery device.

71. A kit comprising a container comprising a solution of niclosamide, or a pharmaceutically acceptable salt thereof, and polyethylene glycol (PEG); and an inhaler device and/or an intranasal delivery device.

72. The system or kit according to claim 70 or claim 71, wherein the inhaler device or the intranasal delivery device is adapted to aerosolize the solution.

73. The system or kit according to claim 72, wherein the inhaler device or the or an intranasal delivery device is adapted to deliver the aerosolized solution intranasally or intraorally to a subject.

74. The system or kit according to any one of claims 70 to 73, wherein the inhaler device is selected from a jet nebulizer, a vibrating mesh nebulizer, an ultrasonic nebulizer or a pressurised metered dose inhaler (pMDI).

75. An inhalable solution comprising from about 1% to 10% by weight niclosamide ethanolamine and PEG, wherein the PEG has an average molecular weight of less than 600; and optionally a taste-masking agent.

76. An inhalable solution according to claim 75, comprising from about 4.5% to about 6% by weight niclosamide ethanolamine; about 90% to 95.5% PEG 400 and optionally a taste-masking agent.

77. An inhalable solution according to claim 75, comprising from about 4.5% to about 6% by weight niclosamide ethanolamine; about 90% to 95.5% PEG 200 and optionally a taste-masking agent.

78. The inhalable solution according to any one of claims 75 to 77, wherein the solution is a non-aqueous solution, for example a solution containing less than 2% by weight water, preferably less than 0.1% by weight water, more preferably wherein the solution is anhydrous.

79. The composition for use or method according to any one of claims 1 to 78, wherein the subject is hospitalized.

80. The composition for use or method according to any one of claims 1 to 79, wherein the composition is for preventing, or reducing the likelihood of, progression of the disease, e.g. from mild to moderate or from moderate to severe COVID-19.

81. The composition for use or method according to any one of claims 1 to 80, wherein the composition is administered prophylactically.

82. The composition for use or method according to claim 81, wherein the composition is prophylactically administered to a subject who has been, or is suspected as having been, exposed to a person who is diagnosed as being infected with a viral infection.

83. The composition for use or method according to claim 82, wherein the viral infection is SARS-CoV-2.

84. The composition for use or method according to claim 81, wherein the composition is prophylactically administered to a non-infected subject who is at a higher risk from COVID-19.

85. The composition for use or method according to any one of claims 81 to 84, wherein the prophylactic administration is for:
reducing the risk of the subject contracting symptomatic or non-symptomatic COVID-19 infection;
reducing the risk of mortality from COVID-19;
reducing the severity of symptoms of COVID-19; and/or
reducing the risk of the subject contracting moderate or severe COVID-19.

86. The composition for use or method according to any one of claims 81 to 84, wherein the prophylactic administration is for:
reducing the risk of the subject contracting a secondary infection (e.g. a secondary bacterial infection
reducing the risk of mortality from a secondary infection; and/or
reducing the severity of a secondary infection.

87. The composition for use or method according to claim 84, wherein the subject has an existing condition or disease, such as: such as diabetes, cancer, heart disease, hypertension, cerebrovascular disease, SCID, sickle cell disease, thalassemia, pulmonary fibrosis, interstitial lung disease, chronic lung disease such as COPD, asthma and cystic fibrosis, emphysema, bronchitis, chronic kidney disease, chronic liver disease, hepatitis, autoimmune disease, a condition affecting the brain or nerves, a muscle wasting condition, or a severe or profound learning disability.

88. The composition for use or method according to claim 84, wherein the subject is selected from: subjects who have had a body tissue (e.g. an organ) transplant; subjects who have had an organ (e.g. spleen) removed; subjects receiving (or who have received) chemotherapy, immunotherapy, antibody therapy or radiotherapy; subjects receiving (or who have received) cancer treatment; subjects receiving (or who have received) protein kinase inhibitors or PARP inhibitors; subjects who have had a blood, bone marrow or stem cell transplant; subjects taking immunosuppressants; subjects with HIV or AIDS; and subjects on haemodialysis.

89. The composition for use or method according to any preceding claim, wherein the composition is for use in the treatment of a viral infection in an asymptomatic subject, optionally wherein the viral infection is SARS-CoV-2.

90. The composition for use or method according to any preceding claim, wherein the treatment is for:
reducing or eliminating the viral load in the subject;
accelerating seroconversion in the subject;
reducing inter-subject transmission of the viral;
reducing viral shedding;
preventing or reducing the risk of the subject developing symptoms; and/or
preventing or reducing the risk of progression of disease.

91. The composition for use or method according to any preceding claim, wherein the composition is administered intranasally.
